SEQUENCE LISTING (90 sequences)

SEQ ID NO: 1 - DNA, 21 bp, Artificial Sequence
  Synthetic primer
cccagggacctctctctaatc

SEQ ID NO: 2 - DNA, 21 bp, Artificial Sequence
  synthetic primer
atgggctacaggcttgtcact

SEQ ID NO: 3 - DNA, 20 bp, Artificial Sequence
  synthetic primer
agcacagaaagcatgatccg

SEQ ID NO: 4 - DNA, 20 bp, Artificial Sequence
  synthetic primer
gccacaagcaggaatgagaa

SEQ ID NO: 5 - DNA, 24 bp, Artificial Sequence
  synthetic primer
ctaattattcggtaactgacttga

SEQ ID NO: 6 - DNA, 21 bp, Artificial Sequence
  synthetic primer
acagttcagccatcacttgga

SEQ ID NO: 7 - DNA, 21 bp, Artificial Sequence
  synthetic primer
gacaatcaggccatcagcaac

SEQ ID NO: 8 - DNA, 22 bp, Artificial Sequence
  synthetic primer
cggatgagctcattgaatgctt

SEQ ID NO: 9 - DNA, 20 bp, Artificial Sequence
  synthetic primer
acacagacagccactcacct

SEQ ID NO: 10 - DNA, 20 bp, Artificial Sequence
  synthetic primer
ttctgccagtgcctctttgc

SEQ ID NO: 11 - DNA, 21 bp, Artificial Sequence
  synthetic primer
ccttcttgggactgatgctgg

SEQ ID NO: 12 - DNA, 21 bp, Artificial Sequence
  synthetic primer
gcctccgacttgtgaagtggt

SEQ ID NO: 13 - DNA, 20 bp, Artificial Sequence
  synthetic primer
aaaccaccggaaggaaccat

SEQ ID NO: 14 - DNA, 22 bp, Artificial Sequence
  synthetic primer
ccttcacacagagctgcagaaa

SEQ ID NO: 15 - DNA, 21 bp, Artificial Sequence
  synthetic primer
ccactctcaagggcggtcaaa

SEQ ID NO: 16 - DNA, 21 bp, Artificial Sequence
  synthetic primer
tacgatccaggcttcccgggt

SEQ ID NO: 17 - DNA, 20 bp, Artificial Sequence
  synthetic primer
gtgtgggactgaagcacctg

SEQ ID NO: 18 - DNA, 20 bp, Artificial Sequence
  synthetic primer
gtctcccaaggcattctccc

SEQ ID NO: 19 - DNA, 20 bp, Artificial Sequence
  synthetic primer
agaagagaccacggcagaag

SEQ ID NO: 20 - DNA, 20 bp, Artificial Sequence
  synthetic primer
ccttggaccaggttcagtgt

SEQ ID NO: 21 - DNA, 21 bp, Artificial Sequence
  synthetic primer
gcctgttcctgtgatgtggag

SEQ ID NO: 22 - DNA, 25 bp, Artificial Sequence
  synthetic primer
tgcccacagacattcatacagtttc

SEQ ID NO: 23 - DNA, 20 bp, Artificial Sequence
  synthetic primer
cacagctctggagatggtga

SEQ ID NO: 24 - DNA, 20 bp, Artificial Sequence
  synthetic primer
ggtcccacatattccctcct

SEQ ID NO: 25 - DNA, 21 bp, Artificial Sequence
  synthetic primer
acagatgaagtgctccttcca

SEQ ID NO: 26 - DNA, 21 bp, Artificial Sequence
  synthetic primer
gtcggagatttcgtactggat

SEQ ID NO: 27 - DNA, 20 bp, Artificial Sequence
  synthetic primer
tcacagcagcacatcaacaa

SEQ ID NO: 28 - DNA, 20 bp, Artificial Sequence
  synthetic primer
tgtcctcatcctggaaggtc

SEQ ID NO: 29 - DNA, 23 bp, Artificial Sequence
  synthetic primer
cggcctctatttgaagatatgac

SEQ ID NO: 30 - DNA, 20 bp, Artificial Sequence
  synthetic primer
ccatacctctaggctggcta

SEQ ID NO: 31 - DNA, 20 bp, Artificial Sequence
  synthetic primer
acaactttggccgacttcac

SEQ ID NO: 32 - DNA, 20 bp, Artificial Sequence
  synthetic primer
gggttcactggcactttgat

SEQ ID NO: 33 - DNA, 23 bp, Artificial Sequence
  synthetic primer
gggtgatagaagaggccaagact

SEQ ID NO: 34 - DNA, 21 bp, Artificial Sequence
  synthetic primer
agctcctgcaactcctcaaga

SEQ ID NO: 35 - DNA, 21 bp, Artificial Sequence
  synthetic primer
tgaaggaggccaccaaggagg

SEQ ID NO: 36 - DNA, 21 bp, Artificial Sequence
  synthetic primer
agaggtcacccaggtagcggg

SEQ ID NO: 37 - DNA, 21 bp, Artificial Sequence
  synthetic primer
tgatgacatcaagaaggtggt

SEQ ID NO: 38 - DNA, 21 bp, Artificial Sequence
  synthetic primer
tccttggaggccatgtgggcc

SEQ ID NO: 39 - DNA, 20 bp, Artificial Sequence
  synthetic primer
caggcattgctgacaggatg

SEQ ID NO: 40 - DNA, 20 bp, Artificial Sequence
  synthetic primer
tgctgatccacatctgctgg

SEQ ID NO: 41 - DNA, 24 bp, Artificial Sequence
  synthetic primer
cagtgtgccatctacccatgctta

SEQ ID NO: 42 - DNA, 22 bp, Artificial Sequence
  synthetic primer
ggtgcttggttcaaattgagcc

SEQ ID NO: 43 - DNA, 22 bp, Artificial Sequence
  synthetic primer
tgggatatggagcttctatcgc

SEQ ID NO: 44 - DNA, 22 bp, Artificial Sequence
  synthetic primer
ggtgagtcgaaacacggatcat

SEQ ID NO: 45 - DNA, 24 bp, Artificial Sequence
  synthetic primer
actcctgtcttggttgggtctgat

SEQ ID NO: 46 - DNA, 22 bp, Artificial Sequence
  synthetic primer
cgtgctccgcttattcctttct

SEQ ID NO: 47 - DNA, 22 bp, Artificial Sequence
  synthetic primer
ctcttcacgaaatccagcagca

SEQ ID NO: 48 - DNA, 22 bp, Artificial Sequence
  synthetic primer
ccatgaggtttcaagtgcttgg

SEQ ID NO: 49 - DNA, 20 bp, Artificial Sequence
  synthetic primer
tgaaagcagaacctggtcca

SEQ ID NO: 50 - DNA, 20 bp, Artificial Sequence
  synthetic primer
cactgttcatgcttggtgca

SEQ ID NO: 51 - DNA, 22 bp, Artificial Sequence
  synthetic primer
cagaacatcaagccacggaaga

SEQ ID NO: 52 - DNA, 24 bp, Artificial Sequence
  synthetic primer
aaagttgtctcaggtcctccttcc

SEQ ID NO: 53 - DNA, 22 bp, Artificial Sequence
  synthetic primer
cgtggagatcatagccaacgat

SEQ ID NO: 54 - DNA, 20 bp, Artificial Sequence
  synthetic primer
attccaagtgcgtccgatga

SEQ ID NO: 55 - DNA, 20 bp, Artificial Sequence
  synthetic primer
gggaggtcaccttcaagtcg

SEQ ID NO: 56 - DNA, 19 bp, Artificial Sequence
  synthetic primer
ctcgaggtgcagatgtggg

SEQ ID NO: 57 - DNA, 23 bp, Artificial Sequence
  synthetic primer
aaaggttccgctcatacaaaggg

SEQ ID NO: 58 - DNA, 23 bp, Artificial Sequence
  synthetic primer
tgaatgaagccagcaggaagttg

SEQ ID NO: 59 - DNA, 24 bp, Artificial Sequence
  synthetic primer
aagccttgcagagagggtattgac

SEQ ID NO: 60 - DNA, 24 bp, Artificial Sequence
  synthetic primer
gcagttagagcatgactggcagat

SEQ ID NO: 61 - DNA, 20 bp, Artificial Sequence
  synthetic primer
tcgtggaatggtaatcttgc

SEQ ID NO: 62 - DNA, 20 bp, Artificial Sequence
  synthetic primer
cctcccgtttctctatcacg

SEQ ID NO: 63 - DNA, 22 bp, Artificial Sequence
  synthetic primer
aaggctgttggagctgatagca

SEQ ID NO: 64 - DNA, 22 bp, Artificial Sequence
  synthetic primer
cacccacatgttaatgttgccc

SEQ ID NO: 65 - DNA, 22 bp, Artificial Sequence
  synthetic primer
taactgcatctccatgctggtg

SEQ ID NO: 66 - DNA, 22 bp, Artificial Sequence
  synthetic primer
tggcgtagagggtgtcaaactt

SEQ ID NO: 67 - DNA, 24 bp, Artificial Sequence
  synthetic primer
tcgcctagtaagacaggaccatca

SEQ ID NO: 68 - DNA, 20 bp, Artificial Sequence
  synthetic primer
tggttccatttcggcaacct

SEQ ID NO: 69 - DNA, 24 bp, Artificial Sequence
  synthetic primer
agcacaggcacagaagacaagagt

SEQ ID NO: 70 - DNA, 24 bp, Artificial Sequence
  synthetic primer
aatgtgtccagtcatcgtctcctc

SEQ ID NO: 71 - DNA, 21 bp, Artificial Sequence
  synthetic primer
ctggtgaatgggcacagcatg

SEQ ID NO: 72 - DNA, 21 bp, Artificial Sequence
  synthetic primer
cgtccgctggtaacatccctt

SEQ ID NO: 73 - DNA, 20 bp, Artificial Sequence
  synthetic primer
tgaaatcaatgctgcctggg

SEQ ID NO: 74 - DNA, 24 bp, Artificial Sequence
  synthetic primer
ccagaacagtataacggcaactcc

SEQ ID NO: 75 - DNA, 21 bp, Artificial Sequence
  synthetic primer
ttacgggagaaaactcagggc

SEQ ID NO: 76 - DNA, 22 bp, Artificial Sequence
  synthetic primer
gggtccaacttgtccagaatgc

SEQ ID NO: 77 - DNA, 22 bp, Artificial Sequence
  synthetic primer
tggagcagcaagtggtggattt

SEQ ID NO: 78 - DNA, 22 bp, Artificial Sequence
  synthetic primer
tgtccattcccaagcgtgttct

SEQ ID NO: 79 - DNA, 18 bp, Artificial Sequence
  synthetic primer
gagatttctccaacccag

SEQ ID NO: 80 - DNA, 19 bp, Artificial Sequence
  synthetic primer
tctgactttcggaggtact

SEQ ID NO: 81 - DNA, 20 bp, Artificial Sequence
  synthetic primer
ctacaggcctgggcttacct

SEQ ID NO: 82 - DNA, 20 bp, Artificial Sequence
  synthetic primer
gccacaagcaggaatgagaa

SEQ ID NO: 83 - DNA, 21 bp, Artificial Sequence
  synthetic primer
tctgccaactaccgagcctat

SEQ ID NO: 84 - DNA, 21 bp, Artificial Sequence
  synthetic primer
ctcttctgcctctcgttccat

SEQ ID NO: 85 - DNA, 17 bp, Artificial Sequence
  synthetic primer
cattcctggatgtgctg

SEQ ID NO: 86 - DNA, 18 bp, Artificial Sequence
  synthetic primer
gaacaagacgcaggcttt

SEQ ID NO: 87 - DNA, 21 bp, Artificial Sequence
  synthetic primer
atgaatctcagaactcaacac

SEQ ID NO: 88 - DNA, 22 bp, Artificial Sequence
  synthetic primer
aaacttgaatatttggtgggga

SEQ ID NO: 89 - DNA, 19 bp, Artificial Sequence
  synthetic primer
ctggccgccgagaagctag

SEQ ID NO: 90 - DNA, 20 bp, Artificial Sequence
  synthetic primer
ccccaccatgatggactgta

(19) United States
(12) Patent Application Publication
(10) Pub. No.: US 2021/0121482 A1
(43) Pub. Date: Apr. 29, 2021

(54) WITHANOLIDES FOR THE TREATMENT OF CACHEXIA

(71) Applicant: University of Louisville Research Foundation, Inc., Louisville, KY (US)

(72) Inventors: Sham S. Kakar, Prospect, KY (US); Alex R. Straughn, Sellersburg, IN (US); Natia Q. Kelm, Louisville, KY (US)

(21) Appl. No.: 17/082,430

(22) Filed: Oct. 28, 2020

Related U.S. Application Data
(60) Provisional application No. 62/927,418, filed on Oct. 29, 2019.

(51) Int. Cl. A61K 31/585; A61P 35/00; A61P 9/00; A61K 36/81; A61P 21/00

(52) U.S. Cl. CPC A61K 31/585; A61P 35/00; A61P 21/00; A61K 36/81; A61P 9/00

(57) ABSTRACT

A method of ameliorating cachexia in a subject in need thereof comprising administering to the subject a therapeutically effective amount of a steroidal lactone is provided. In particular, the steroidal lactone may be withanolide A (WFA) or derivatives thereof. In particular, the method of treatment inhibits development of cachexia associated with ovarian cancer, as well as the induction of cardiac atrophy. Administration of WFA modulates expression of genes that activate satellite cells and inhibit myofibrillar atrophy.

### CROSS-REFERENCE TO RELATED APPLICATIONS

This application claims benefit of U.S. Provisional Application No. 62/927,418, filed Oct. 29, 2019, the complete contents of which is hereby incorporated by reference.

### STATEMENT OF FEDERALLY SPONSORED RESEARCH AND DEVELOPMENT

This invention was made with government support under Grant Numbers U01 CA18514 and T32 HL134644 awarded by the National Institutes of Health (NIH). The government has certain rights in the invention.

### FIELD OF THE INVENTION

The invention relates to the use of withanolides such as withanolide A (also known as withaferin A) for the treatment of cachexia, for example, cachexia induced by cancer. The invention further relates to the use of withanolides for the treatment of cachexia and disruption of the renin-angiotensin system and tumor angiogenesis that is associated with cachexia.

### BACKGROUND

Cachexia is a multifactorial disorder and complex metabolic syndrome that is primarily characterized by a significant loss and/or weakening of skeletal muscle. It sometimes involves the loss of adipose tissue, but it is usually to a lesser extent than the loss of skeletal muscle. Depending on the oncological setting, cachexia is exhibited in up to 80% of cancer patients and is the primary cause of mortality in up to 30% of cancer patients. Cachexia is highly correlated with a poor clinical prognosis, a decrease in quality of life, and a tolerance to antineoplastic agents. Currently, there is no therapeutic treatment for cachexia.

Ovarian cancer is one of the leading causes of cancer mortality in the US because this disease is typically diagnosed in advanced stages with widespread metastases. For average risk patients, no screening tests are available for diagnosis at early stages. Therefore, very soon after diagnosis, patients experience the clinical symptoms of cachexia: involuntary body weight loss, severe muscle wasting, fatigue, and a decreased response to anticancer therapies; these symptoms lead to a reduction in quality of life and overall survival rate. Ovarian cancer patients frequently exhibit cachexia, which is primarily marked by a significant loss of skeletal muscle and functional muscle weakening. Development and prognosis of chronic heart failure are related to poor nutritional status as a result of cachexia. The prevalence of cardiac cachexia ranges from 10% to 39%, depending on the disease state. The prognosis for cancer patients with cardiac cachexia is poor, with mortality reaching up to 50% in 18 months. Several cancers have been demonstrated to have a deleterious effect on the heart, but common cancer treatments, such as chemo- and/or radiotherapy, are capable of inducing a cachectic phenotype in and of themselves or exacerbating cardiac dysfunction stemming from the cancer.

Skeletal muscle mass is primarily regulated by myogenic progenitors, the rate of protein degradation, and the rate of protein synthesis. Satellite cells are the primary myogenic progenitors responsible for the majority of skeletal muscle regeneration and have been shown to be spuriously activated in an NF-κB-dependent manner in multiple models of cancer-induced cachexia. For example, see He et al. J Clin Invest. 2013; 123(11):4821-35. He et al. showed an increase in proliferating satellite cells in the settings of cancer-induced cachexia but simultaneously demonstrated that they are functionally inactivated (i.e. did not differentiate/fuse with muscle to repair injury) through a Pax7-dependent downregulation of MyoD, leading to a failure in muscle repair.

Additionally, cancer-induced cachexia has been reported to upregulate various branches of the unfolded protein response (UPR), exerting deleterious effects on muscle mass. The protein kinase R-like endoplasmic reticulum kinase (PERK) arm of the UPR is required for both the survival and differentiation of satellite cells to facilitate proper muscle repair. However, overactivation of the UPR is known to result in skeletal muscle atrophy through activation of proteolytic systems. Upregulation of both the ubiquitin proteasome system (UPS) and autophagy-lysosomal system (ALS) have been observed in skeletal muscle in the settings of cancer-induced cachexia, facilitating proteolytic degradation of proteins culminating in the atrophy of muscle. In addition to protein degradation modalities, the UPR also acts to limit the rate of protein synthesis through an inhibition in translation and regulation of the Akt/mTOR pathway, attenuating muscle mass.

Myocardial atrophy is another common feature observed in murine models of cancer-induced cachexia, with a decrease in heart weight of up to ˜20% in tumor-bearing mice compared to that of non-tumor-bearing mice. However, the same study also showed that ectopic implantation of C26 colon carcinoma cells into female mice yielded a milder atrophying effect due to the cardioprotective effects of estrogen. Along similar lines, post-menopausal women have an increased risk of cardiovascular disease due to the loss of endogenous estrogen production. A majority of ovarian cancer patients are post-menopausal with very low levels of circulating estrogen. In addition, some xenograft models of ovarian cancer resulted in the dysregulation of the estrous cycle and/or premature termination of estrous cycling, resulting in decreased levels of circulating estrogen.

In addition to complications due to wasting of skeletal muscle and cardiac atrophy due to cachexia, the renin-angiotensin system (RAS) is disrupted in patients with ovarian cancer. The RAS is known for its crucial role in maintaining a healthy cardiovascular system, as well as fluid and electrolyte balance. Angiotensin II (Ang II), the principal effector of the RAS, binds to the two distinct Ang II receptors: Ang II type 1 (AT₁R) and Ang II type 2 (AT₂R). Ang II stimulates angiogenesis via the upregulation of vascular endothelial growth factor, and these Ang II-induced cellular effects are mostly mediated through the specific G protein-coupled AT₁R. AT₂R is highly expressed in the fetus, rapidly reduced after birth and is upregulated in response to pathologic stimuli to have cardioprotective effect, such as hypertension and/or myocardial ischemia. However, in patients with ovarian cancer, expression of AT₁R correlates with tumor angiogenesis and poor clinical outcomes.

Thus, there is a need to intervene in the complex and multifactorial effects of ovarian cancer, including cachexia and the effects of cachexia on RAS that complicate the treatment of ovarian cancer. There is no satisfactory treatment that addresses cachexia, cardiac cachexia and tumor angiogenesis in response to elevated expression of AT₁R.

### SUMMARY

The invention is a method of ameliorating cachexia in a subject in need thereof, comprising administering to the subject a therapeutically effective amount of a steroidal lactone. The steroidal lactone can be a withanolide or a derivative thereof. In one embodiment, the withanolide is isolated from Withania somnifera. In one embodiment, the withanolide is a derivative or analog of withanolide. In yet another embodiment, the withanolide is withanolide A or withaferin A (WFA).

Typically, the cachexia is induced by a disorder such as a cancer, a neurological disorder, HIV infection, AIDS, sepsis, a chronic pulmonary disease, or a cardiac disorder. In one embodiment, the cancer is an ovarian cancer. In another embodiment, the cachexia is cardiac cachexia. In yet another embodiment. the invention is a method of reducing myofibrillar atrophy and/or conversion of type IIA myofibers to type IIB myofibers in a subject in need thereof, comprising administering to the subject a therapeutically effective amount of a steroidal lactone, such as a WFA, a derivative of WFA or an analog of WFA. The subject who is treated has myofibrillar atrophy and/or myofiber-type conversion in skeletal and/or cardiac muscle that may arise in association with a cancer, or independent of cancer. Thus, in one embodiment, the invention is a method of attenuating arrhythmia, systolic dysfunction, and/or diastolic dysfunction in the subject. In another embodiment, the invention is a method of activating satellite cells.

In another embodiment, the invention is a method of ameliorating cachexia in a subject in need thereof, comprising administering to the subject a therapeutically effective amount of a steroidal lactone. The steroidal lactone can be a withanolide or a derivative thereof. In one embodiment, the withanolide is isolated from Withania somnifera. In one embodiment, the withanolide is a derivative or analog of withanolide. In yet another embodiment, the withanolide is withanolide A (also known as withaferin A; WFA).

Typically, the cachexia is induced by a disorder such as a cancer, a neurological disorder, HIV infection, AIDS, sepsis, a chronic pulmonary disease, and a cardiac disorder. In one embodiment, the cancer is an ovarian cancer. In another embodiment, the cachexia is cardiac cachexia. Thus, another embodiment of the invention is a method of reducing myofibrillar atrophy and/or conversion of type IIA myofibers to type IIB myofibers in a subject in need thereof comprising administering to the subject a therapeutically effective amount of a steroidal lactone, such as a WFA, a derivative of WFA or an analog of WFA. The subject who is treated has myofibrillar atrophy and/or myofiber-type conversion in skeletal and/or cardiac muscle that may arise in association with a cancer, or independent of cancer. Thus, the method of the invention is directed to attenuating arrhythmia, systolic dysfunction, and/or diastolic dysfunction in the subject.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings, which are incorporated in and constitute a part of this specification, illustrate embodiments of the invention and, together with a general description of the invention given above, and the detailed description given below, serve to explain the invention.

FIGS. 1A-1F show that WFA decreases the mortality rate and body composition changes associated with ovarian cancer. (1A) Body weight fold change of control, vehicle treated, WFA 2 mg/kg, and WFA 6 mg/kg groups over period of study. N=5 in each group. (1B) Kaplan-Meier survival analysis. (1C) Representative DEXA scan images of control, vehicle treated, WFA 2 mg/kg, and WFA 6 mg/kg mice. Area within dashed box surrounding image is the same. Quantitative estimation of whole body DEXA scan minus head ROI of (1D) adipose tissue and (1E) lean tissue as a percentage of body weight. (1F) Average weight of free peritoneal tumor(s). n=5 (control), 4 (vehicle treated), 5 (WFA 2 mg/kg), and 4 (WFA 6 mg/kg). *P<0.05; **p<0.01; ***p<0.001, value significantly different from corresponding value of control group as determined by one-way ANOVA followed by Tukey's HSDT. ^{#}P<0.05, value significantly different from corresponding value of vehicle treated group. ^{$}P<0.05, value significantly different from corresponding value of WFA 2 mg/kg group.

FIGS. 2A-2E show that WFA ameliorates muscle strength loss and myofiber size reduction in the settings of ovarian cancer-induced cachexia. Quantification of mean (2A) forelimb and (2B) total limb grip strength normalized to body weight at time of assessment. N=5 in all groups. (2C) Quantification of normalized tibialis anterior (TA), gastrocnemius (GA), and quadriceps femoris (QF) muscle weights to total final body weight. (2D) Representative images of H&E stained GA and QF transverse muscle sections. (2E) Quantification of average myofiber cross-sectional area (CSA). Scale Bar=50 μm. N=5 (control), 4 (vehicle treated), 5 (WFA 2 mg/kg), and 4 (WFA 6 mg/kg). *P<0.05; **p<0.01; ***p<0.001; ****p<0.0001, value significantly different from corresponding value of control mice as determined by one-way ANOVA followed by Tukey's HSDT. ^{#}P<0.05, value significantly different from corresponding value of vehicle treated group. ^{$}P<0.05, value significantly different from corresponding value of WFA 2 mg/kg group.

FIGS. 3A-3D show that WFA inhibits a slow to fast fiber-type conversion in ovarian cancer. (3A) Representative images of transverse TA muscle sections from control, vehicle treated, WFA 2 mg/kg, and WFA 6 mg/kg mice subjected to triple immunostaining against MyHC I, IIa, and IIb proteins. Unstained fibers were considered to be Type IIx. Scale Bar=50 μm. (3B) Quantification of the percentage of Type I, Type IIa, Type IIx, and Type IIb myofibers in TA muscle. Relative mRNA levels of (3C) select inflammatory cytokines and (3D) NLRP3 inflammasome markers in TA muscle. N=4 in each group. *P<0.05; **p<0.01; ***p<0.001; ****p<0.0001, value significantly different from corresponding value of control group as determined by one-way ANOVA followed by Tukey's HSDT. ^{#}P<0.05, value significantly different from corresponding value of vehicle treated group. ^{$}P<0.05, value significantly different from corresponding value of WFA 2 mg/kg group.

FIGS. 4A-4D show that WFA impedes activation of NF-κ-related inflammatory cytokines. (4A) Representative immunoblots of NF-κB proteins phospho- and total p65, and unrelated protein β-Actin in vehicle treated, WFA 2 mg/kg, and WFA 6 mg/kg groups. N=3 in each group. (4B) Representative images of tumor samples subjected to immunostaining against phospho-p65. Nuclei were visualized with DAPI counterstaining. Arrow points to phospho-p65⁺ nuclei. Scale Bar=20 μm. N=4, 5, 4, respectively. (4C) Relative mRNA levels of select inflammatory cytokines in tumor samples. N=3 in each group. (4D) Concentration of select cytokines (pg/mL) in tumor samples. N=4 in each group. ^{#}P<0.05; ^{##}p<0.01; ^{###}p<0.001; ^{####}p<0.0001, value significantly different from corresponding value of vehicle treated group as determined by one-way ANOVA followed by Tukey's HSDT. ^{$}P<0.05, value significantly different from corresponding value of WFA 2 mg/kg group.

FIGS. 5A and 5B show that WFA reduces signaling through the NLRP3 inflammasome. (5A) Relative mRNA levels of NLRP3 and CASP1 in tumor samples of vehicle treated, WFA 2 mg/kg, and WFA 6 mg/kg groups. N=3 in each group. (5B) Representative images of tumor samples subjected to immunostaining against Caspase 1 and NLRP3. Nuclei were visualized with DAPI counterstaining. Arrow points to cells that are Caspase 1+ and NLRP3⁺. Scale Bar=20 μm. N=4 (vehicle treated), 5 (WFA 2 mg/kg), and 4 (WFA 6 mg/kg). ^{#}P<0.05; ^{##}p<0.01; ^{###}p<0.001; ^{####}p<0.0001, value significantly different from corresponding value of vehicle treated group as determined by one-way ANOVA followed by Tukey's HSDT. ^{$}P<0.05, value significantly different from corresponding value of WFA 2 mg/kg group.

FIGS. 6A-6C show that WFA impairs execution of NLRP3 inflammasome signaling. (6A) Representative images of tumor samples subjected to immunostaining against IL-1β, IL-18, and HO-1. Nuclei were visualized with DAPI counterstaining. Arrow points to cells that are IL-1β⁺, IL-18⁺, and HO-1⁺. Arrowhead points to cells that are IL-1β⁺, IL-18⁺, and HO-1⁻. Scale Bar=20 μm. N=4 (vehicle treated), 5 (WFA 2 mg/kg), and 4 (WFA 6 mg/kg). (6B) Relative mRNA levels of IL-1β, IL-18, and HO-1 in tumor samples of vehicle treated, WFA 2 mg/kg, and WFA 6 mg/kg groups. N=3 in each group. (6C) Concentration of inflammasome-related cytokines (pg/ml) in tumor samples. N=4 in each group. ^{#}P<0.05; ^{##}p<0.01; ^{###}p<0.001, value significantly different from corresponding value of vehicle treated group as determined by one-way ANOVA followed by Tukey's HSDT. ^{$}P<0.05, value significantly different from corresponding value of WFA 2 mg/kg group.

FIG. 7 shows the effect of WFA on ovarian cancer-induced cachexia illustrated as a mechanistic model of the effect of WFA effect on upstream cachectic signaling in ovarian cancer.

FIG. 8 shows an experimental design for assessment of body weight, GSL, and heart function after WFA treatment.

FIGS. 9A and 9B show (9A) forelimb grip strength and (9B) total grip strength in tumor-free and tumor-bearing mice on day 1.

FIGS. 10A and 10B show (10A) forelimb grip strength and (10B) total grip strength in tumor-free and tumor-bearing mice after week 1.

FIGS. 11A and 11B show (11A) forelimb grip strength and (11B) total grip strength in tumor-free and tumor-bearing mice after week 2.

FIGS. 12A and 12B show (12A) forelimb grip strength and (12B) total grip strength in tumor-free and tumor-bearing mice after week 3.

FIGS. 13A and 13B show (13A) forelimb grip strength and (13B) total grip strength in tumor-free and tumor-bearing mice after week 4.

FIGS. 14A-14F show that WFA increases muscle mass under tumor-free and tumor-bearing conditions. Quantification of normalized muscle mass in the (14A) TA, (14B) GA, and (14C) QF muscles. (14D) Representative images of H&E-stained tibialis anterior (TA) transverse muscle sections. Scale bar=50 μm. Inset images magnified from whole image. Quantification of average (14E) myofiber cross-sectional area (CSA) and (14F) minimal Feret's diameter in TA muscle. N=10 in all groups. *p<0.05; **p<0.01; ***p<0.001; ****p<0.0001, value significantly different from corresponding value of tumor-free vehicle-treated group by two-way ANOVA followed by Tukey's multiple comparison test post hoc analysis. ^{#}p<0.05, value significantly different from corresponding value of tumor-free WFA 2 mg/kg group. ^{$}p<0.05, value significantly different from corresponding value of tumor-free WFA 4 mg/kg group. ^{@}p<0.05, value significantly different from corresponding value of tumor-bearing vehicle-treated group.

FIGS. 15A-15F show that WFA activates satellite cells to proliferate and differentiate. (15A) Representative images of transverse TA muscle sections after immunostaining for Pax7 (red color), MyoD (yellow color), and Laminin (green color) proteins in tumor-free and tumor-bearing groups. Nuclei were identified by counterstaining with DAPI (blue color). Scale bar=25 μm. Quantification of (15B) the total number of Pax7⁺ cells per Laminin⁺ myofiber, (15C) the proportion of Pax7⁺/MyoD⁻ cells, and (15D) the proportion of Pax7⁺/MyoD⁺ cells. Solid white arrow denotes Pax7⁺/MyoD⁻ cells. Solid yellow arrow denotes Pax7⁺/MyoD⁺ cells. Dashed yellow arrow denotes Pax7⁻/MyoD⁺ cells. Relative mRNA levels of (15E) Pax7 and (15F) Myod1 in GA muscle. N=10 in all groups. *p<0.05; **p<0.01; ***p<0.001; ****p<0.0001, value significantly different from corresponding value of tumor-free vehicle-treated group by two-way ANOVA followed by Tukey's multiple comparison test post hoc analysis. ^{#}p<0.05, value significantly different from corresponding value of tumor-free WFA 2 mg/kg group. ^{$}p<0.05, value significantly different from corresponding value of tumor-free WFA 4 mg/kg group. ^{@}p<0.05, value significantly different from corresponding value of tumor-bearing vehicle-treated group.

FIGS. 16A-16J show that WFA and ovarian cancer differentially activate the Unfolded Protein Response (UPR) signaling pathway. (16A-16H) Relative mRNA levels of select markers of the UPR [(16A) Eif2ak3, (16B) Atf4, (16C) Ddit3, (16D) Ppp1r15a, (16E) Hspa5, (16F) Hsp90B1, (16G) Ern1, and (16H) Tnfrsf10b] in the GA muscle of tumor-free and tumor-bearing groups. (16I) Representative immunoblots for UPR signaling proteins. (16J) Representative images from PCR amplification and resolution via agarose gel electrophoresis of unspliced, spliced, and total XBP-1, and beta-actin. *p<0.05; **p<0.01; ***p<0.001; ****p<0.0001, value significantly different from corresponding value of tumor-free vehicle-treated group by two-way ANOVA followed by Tukey's multiple comparison test post hoc analysis. ^{#}p<0.05, value significantly different from corresponding value of tumor-free WFA 2 mg/kg group. ^{$}p<0.05, value significantly different from corresponding value of tumor-free WFA 4 mg/kg group. ^{@}p<0.05, value significantly different from corresponding value of tumor-bearing vehicle-treated group.

FIGS. 17A-17E show that WFA inhibits activation of the Ubiquitin Proteasome System (UPS) to preserve muscle mass. (17A-17D) Relative mRNA levels of select E3 ubiquitin ligases associated with cachexia [(17A) Fbxo30, (17B) Fbxo32, (17C) Trim63, and (17D) Traf6]. (17E) Representative immunoblots for poly-ubiquitinated proteins and beta-actin. *p<0.05; **p<0.01; ***p<0.001; ****p<0.0001, value significantly different from corresponding value of tumor-free vehicle-treated group by two-way ANOVA followed by Tukey's multiple comparison test post hoc analysis. ^{#}p<0.05, value significantly different from corresponding value of tumor-free WFA 2 mg/kg group. ^{$}p<0.05, value significantly different from corresponding value of tumor-free WFA 4 mg/kg group. ^{@}p<0.05, value significantly different from corresponding value of tumor-bearing vehicle-treated group.

FIGS. 18A-18D show that WFA inhibits activation of thee Autophagy-Lysosomal System (ALS) to inhibit cachexia. (18A-18C) Relative mRNA levels of critical regulators of the ALS known to be associated with the induction of cachexia [(18A) Sqstm1, (18B) Map11c3b, and (18C) Becn1]. (18D) Representative immunoblots for a LC3B and GAPDH. *p<0.05; **p<0.01; ***p<0.001; ****p<0.0001, value significantly different from corresponding value of tumor-free vehicle-treated group by two-way ANOVA followed by Tukey's multiple comparison test post hoc analysis. ^{#}p<0.05, value significantly different from corresponding value of tumor-free WFA 2 mg/kg group. ^{$}p<0.05, value significantly different from corresponding value of tumor-free WFA 4 mg/kg group. ^{@}p<0.05, value significantly different from corresponding value of tumor-bearing vehicle-treated group.

FIG. 19A-19E shows that WFA reduces ovarian tumor burden and inhibits body weight loss. (19A) Absolute change in tumor-free body weight (i.e. Terminal BW-Tumor Mass-Initial BW) in tumor-free and tumor-bearing mice that were treated with WFA or vehicle. (19B) and quantification of free peritoneal tumor xenografts in WFA- and vehicle-treated groups, (19C) quantification of the bilateral ovaries in WFA- and vehicle-treated groups, and (19D) representative images of free peritoneal tumor xenografts in WFA- and vehicle-treated groups. (19E) Representative images of the bilateral ovaries in WFA- and vehicle-treated groups. N=8-per group. Black circles indicate individual data points. *p<0.05; **p<0.01; ***p<0.001; or ****p<0.0001 indicates a significant difference from the corresponding value of the tumor-free vehicle-treated group by two-way ANOVA followed by Tukey's multiple comparison test. <0.05 indicates a significant difference from the corresponding value of the tumor-free WFA 2 mg/kg group. ^{α}p<0.05 indicates a significant difference from the corresponding value of the tumor-free WFA 4 mg/kg group. ^{¥}p<0.05 indicates a significant difference from corresponding the value of the tumor-bearing vehicle-treated group.

FIGS. 20A-20E show the effects of WFA on left ventricular systolic and diastolic function in the context of ovarian cancer. (20A) Representative M-mode images from all groups. (20B) Systolic function parameters: recorded heart rate via echocardiography (BPM) in tumor-free and tumor-bearing mice that were treated with WFA or vehicle. Graphical representations show (20C) left ventricular % fractional shortening, (20D) cardiac output, and (20E) average calculated LV mass.

FIG. 21A-21C show graphical representations of diastolic function parameters of (21A) E/A ratio, (21B) E/e′ ratio, and (21C) isovolumetric relaxation time (IVRT) in tumor-free and tumor-bearing groups that were treated with WFA or vehicle. N=10 per group. Black circles indicate individual data points. *p<0.05; **p<0.01; ***p<0.001; ****p<0.0001 indicates a significant difference from the corresponding value of the tumor-free vehicle-treated group by two-way ANOVA followed by Tukey's multiple comparison test. ^{#}p<0.05 indicates a significant difference from the corresponding value of the tumor-free WFA 2 mg/kg group. ^{α}p<0.05 indicates a significant difference from the corresponding value of the tumor-free WFA 4 mg/kg group. ^{¥}p<0.05 indicates a significant difference from the corresponding value of the tumor-bearing vehicle-treated group.

FIGS. 22A-22F show that WFA modulates ovarian cancer-induced cardiac atrophy. (22A) Representative 40× images of H&E-stained midventricular heart sections. (22B) Quantification of heart weight normalized to tibial length in tumor-free and tumor-bearing mice that were treated with WFA or vehicle. Scale bar=50 (22C) Quantification of average cardiomyocyte CSA. N=10 per group. Relative mRNA levels of (22D) Tn-I, and (22E) MHCα and (22F) MHCβ. N=5 per group. Black circles indicate individual data points. *p<0.05; **p<0.01; ***p<0.001; ****p<0.0001 indicates a significant difference from the corresponding value of the tumor-free vehicle-treated group by two-way ANOVA followed by Tukey's multiple comparison test. ^{#}p<0.05 indicates a significant difference from the corresponding value of the tumor-free WFA 2 mg/kg group. ^{α}p<0.05 indicates a significant difference from the corresponding value of the tumor-free WFA 4 mg/kg group. ^{¥}p<0.05 indicates a significant difference from the corresponding value of the tumor-bearing vehicle-treated group.

FIGS. 23A and 23B show that WFA diminishes fibrotic scarring in the heart. (23A) Representative 20× images of Masson's trichrome-stained midventricular heart sections in tumor-free and tumor-bearing mice that were treated with WFA or vehicle. Inset images are magnified from the displayed field of view. Scale bar=50 μm. (23B) Quantification of average collagen deposition. N=10 per group. Black circles indicate individual data points. *p<0.05; **p<0.01; ***p<0.001; or ****p<0.0001 indicates a significant difference from the corresponding value of the tumor-free vehicle-treated group by two-way ANOVA followed by Tukey's multiple comparison test. ^{#}p<0.05 indicates a significant difference from the corresponding value of the tumor-free WFA 2 mg/kg group. ^{α}p<0.05 indicates a significant difference from the corresponding value of the tumor-free WFA 4 mg/kg group. ^{¥}p<0.05 indicates a significant difference from the corresponding value of the tumor-bearing vehicle-treated group.

FIGS. 24A-24I show that WFA attenuates circulating Ang II levels and pro-inflammatory cytokines in ovarian cancer. (24A) Levels of Ang II in plasma fractionated from centrally collected blood. (24B) Relative angiotensinogen (AGT) mRNA levels in tumor tissues. Relative transcript levels of (24C) AT_{1A}R and pro-inflammatory cytokines in the heart, including (24D) AT1bR, (24E) AT2R, (24F) TNFα, (24G) IL-6, (24H) MIP-2 and (24I) IFNγ. N=5 per group. Black circles indicate individual data points. *p<0.05; **p<0.01; ***p<0.001; or ****p<0.0001 indicates a significant difference from the corresponding value of the tumor-free vehicle-treated group by two-way ANOVA followed by Tukey's multiple comparison test. ^{#}p<0.05 indicates a significant difference from the corresponding value of the tumor-free WFA 2 mg/kg group. ^{α}p<0.05 indicates a significant difference from the corresponding value of the tumor-free WFA 4 mg/kg group. ^{¥}p<0.05 indicates a significant difference from the corresponding value of the tumor-bearing vehicle-treated group.

FIG. 25 shows a schematic representation of xenografting of ovarian cancer into female mice to induce a cachectic phenotype in cardiac muscle through AT1R that is treated with WFA. Ang II released from tumor induces shift in MHC isoforms from a predominantly adult α-MHC state to one that is primarily embryonic β-MHC in the tumor-bearing vehicle-treated group compared to the tumor-free vehicle-treated group and caused cardiac cachexia. Treatment with WFA reduces Ang II levels and attenuates cardiac cachexic phenotype.

### DETAILED DESCRIPTION

The invention is a therapeutic application of WFA to treat the condition of cachexia. In particular, the invention is well-suited for treatment of cancer-induced cachexia. Cachexia, a common complication of cancer and other chronic disorders, is the involuntary loss of 5% or more of body weight in 6 months or less. Symptoms include weight loss, muscle loss, lack of appetite, fatigue, and decreased strength. Cancer cachexia is recognized as a syndrome with multiple etiologies including systemic inflammation and metabolic dysregulation that results in the muscle atrophy or “wasting” that is the hallmark symptom.

One embodiment of the invention is a method for the treatment of cachexia in a subject in need thereof, comprising administering to the subject a therapeutically effective amount of a steroidal lactone. The steroidal lactone can be a withanolide or a derivative thereof. In one embodiment, the withanolide is isolated from Withania somnifera. In one embodiment, the withanolide is a derivative or analog of withanolide. In yet another embodiment, the withanolide is withanolide A (WFA).

Typically, the cachexia is induced by a disorder such as a cancer, a neurological disorder, HIV infection, AIDS, sepsis, a chronic pulmonary disease, and a cardiac disorder. In one embodiment, the cancer is an ovarian cancer.

In another embodiment, the cachexia is cardiac cachexia. Thus, another embodiment of the invention is a method of reducing myofibrillar atrophy and/or conversion of type IIA myofibers to type IIB myofibers in a subject in need thereof comprising administering to the subject a therapeutically effective amount of a steroidal lactone, such as a WFA, a derivative of WFA or an analog of WFA. The subject having myofibrillar atrophy and/or myofiber-type conversion in skeletal and/or cardiac muscle that may be afflicted with a cancer or may be suffering from a disorder or condition other than cancer.

The steroidal lactone withanolide A (WFA) is a purified extract from the Withania somnifera plant, also known as winter cherry or Ashwagandha. WFA is known for its anti-inflammatory properties and inhibitory effects on cell proliferation and invasion of ovarian cancer [15-19]. Examples of these are found in Fong et al. PloS one 2012, 7(7):e42265; in Kakar et al. Biochem Biophys Res Corn 2012, 423(4):819-825; in Kakar et al. Oncotarget 2017, 8(43):74494-74505, in Kakar et al. PloS one 2014, 9(9):e107596, and in Kakar et al. J Can Stem Cell Res 2016, 4. In addition to its effects on cancer cells, WFA has been shown to target cancer stem cells (see Diffee et al. Am J Physiol Cell Physiol 2002, 283(5):C1376-1382). WFA has also been shown to ameliorate the muscle weakening and myofibrillar atrophy induced by ovarian cancer (see Straughn et al. J Ovarian Res. 2019 Nov. 25; 12(1):115). However, the effect of WFA on cardiac cachexia has not previously been shown.

The therapeutically effective amount of WFA will vary according to the needs of individual subjects. In general, a daily dose of WFA is sufficient to achieve the desired therapeutic effect, however, twice daily doses may be needed for some subjects, while less frequent dosing will be needed for others. In one embodiment of the invention, the therapeutically effective amount is less than 6 mg/kg of body weight. In another embodiment, the WFA doses are in the ranges of 0.2 to 6 mg/kg, 1 to 6 mg/kg, or 2 to 6 mg/kg of body weight are administered. In other embodiments, the WFA doses are in the ranges of 1 to 5 mg/kg of body weight. In other embodiments, the doses of WFA are in the range of 2 to 4 mg/kg of body weight. In general, a dosing regimen comprising repeated dosing allows a lesser amount of WFA to be administered in each dose. In one embodiment, the therapeutically effective doses are injected once and then repeated only as needed. In another embodiment, the therapeutically effective dose is injected, with a second injection after 3 days, and then repeated only as needed. The severity of the cachexia and/or the causative disease can also influence the amount of WFA needed. For example, a daily dose of less than 2 mg/kg or a weekly dose of 2-5 mg/kg will be therapeutically effective for some individuals, while a daily dose of 2-5 mg/kg or more may be required for others.

As will be shown in the Examples of the invention, muscle atrophy is due to a decrease in all myofibrillar proteins, as opposed to a shift specifically in myosin heavy chain (MHC) isoforms. In addition, the Examples will demonstrate that ovarian cancer induces cachectic changes in both the heart and skeletal muscle, and that WFA attenuates cachexia, gross body changes and tumor burden associated with ovarian cancer. Further, xenografting of ovarian cancer into female mice induce a cachectic phenotype in cardiac muscle through AT₁R. Ang II released from tumor induces shift in MHC isoforms from a predominantly adult α-MHC state to one that is primarily embryonic β-MHC in the tumor-bearing vehicle-treated group compared to the tumor-free vehicle-treated group and caused cardiac cachexia. Treatment with WFA reduces Ang II levels and attenuates cachexic phenotype. Furthermore, WFA preserves systolic function in the tumor-bearing mice and partially attenuated diastolic dysfunction.

Skeletal muscle is a major tissue involved in cancer cachexia, accounting for 40% of total body weight loss. Numerous studies characterize mechanisms underlying skeletal muscle atrophy during cancer. However, little attention has been paid to the alteration of myocardial structure and metabolic disorders that occur in the heart. Cardiac cachexia is profoundly understudied. Further, current research in this domain focuses on male lab animals, as the cardioprotective effects of estrogen normally abrogate cardiac effects in the settings of cancer/heart disease. The Examples herein demonstrate induction of cachexia (both skeletal muscle and cardiac) in female animals and attenuate/resolve various pathological parameters associated with the disease state.

Ectopic implantation of C26 colon carcinoma cells is a widely used model of cancer cachexia in rodents. Myocardial atrophy is a common feature observed in this model with a decrease in heart weight reaching ˜20% in tumor-bearing mice. This atrophy seems to be greater in males compared to females (−22 vs. −10% in heart weight 27 days after tumor inoculation). This striking difference in myocardial atrophy could be explained by sexual hormones as inhibition of estrogen receptor signaling totally abolished this protective mechanism.

In the ovarian cancer cell lines SKOV3 and CAOV3, WFA induces cell cycle arrest and the induction of apoptosis, partially through targeting Notch signaling. As will be demonstrated in the Examples herein, WFA leads to autophagic cell death in the ovarian cancer cell line A2780 through increase in reactive oxygen species production and subsequent DNA damage. WFA is also notable due to its capability of targeting and inducing cell death of cancer stem cells, which are frequently spared by traditional chemotherapeutic agents.

Lactones are cyclic carboxylic esters, containing a 1-oxacycloalkan-2-one structure (—(C═O)—O—), or analogues having unsaturation or heteroatoms replacing one or more carbon atoms of the ring. Steroidal compounds have four rings arranged in a specific molecular configuration and generally includes three six-member cyclohexane rings and one five-member cyclopentane ring. “Steroidal lactones” or “lactone steroids” as used herein refer to compounds having a steroid backbone bound to a lactone or one of its derivatives. Exemplary lactone steroids encompassed by the present disclosure include withanolides.

As used herein, the term “withanolide” refers to a natural product or analog thereof having an ergostane (i.e. tetracyclic triterpene) framework in which C-22 and C-26 are appropriately oxidized to form a lactone ring. Withanolides may be isolated from members of family Solanaceae (nightshades). Genera within the nightshade family that have been found to produce withanolides include: Acnistus, Datura, Dunalia, Iochroma, Lycium, Nicandra, Physalis, Salpichroa, Solanum, Withania (e.g. W. somnifera), and Jaborosa. Withanolide natural products may be isolated from the aerial tissue and/or roots. The identity and amounts of natural products isolated is dependent on how the plant is grown. For example, when the plant is grown aeroponically, using chemically-defined nutrient media and without soil, natural products can be isolated. In certain embodiments, the amount of a particular natural product may be altered by growing the plant under different conditions.

Exemplary withanolides include, but are not limited to WFA (4β,5β,6β,22R)-4,27-Dihydroxy-5,6:22,26-diepoxyergosta-2,24-diene-1,26-dione), ixocarpalactone A, nicandrenon-1, and salpichrolide A. Methods for isolating WFA and other withanolides are known in the art, e.g. as described in U.S. Pat. No. 7,108,870 incorporated herein by reference. Embodiments of the disclosure include analogs, derivatives, prodrugs, and pharmaceutically acceptable salts of the compounds of the disclosure as long such compounds retain the functionality of withanolides, e.g. reduce expression of phospho-p65 or pro-inflammatory cytokines such as TNFα, IL-1β, IL-6, and IL-18. Without being bound by theory, upstream regulation of NF-kB signaling is the primary function of withanolides (p65 phosphorylation/translocation) and is an important aspect with regards to cachexia. This allows for a reduction in proinflammatory cytokines that are known to induce myofibrillar atrophy/weakening (and thus cachexia), as well as the unfolded protein response, ubiquitin proteasome system, and autophagy-lysosomal system. However, other signaling mechanisms may be equally valuable in decreasing the tumor bulk. Decreasing the tumor-bulk can in turn reduce proinflammatory cytokines, and subsequently affect cachexia. But, in some instances, the treatment to reduce tumor burden induces a worsening of the cachectic state.

Embodiments of the disclosure include the use of synthesized non-natural analogues of withanolides. As used herein, the term “analogue”, “derivative”, or the like is meant to refer to a change or substitution of an atom with another atom or group. For example, when a hydrogen is replaced with a halogen or a hydroxyl group, such a change produces a derivative. A non-natural product is a compound that is artificially produced or synthesized and not found in nature. The term “synthesized” means that the compound is chemically produced (e.g. in a laboratory) as opposed to being isolated from the natural environment if it is naturally occurring. For example, embodiments of the disclosure include the use of analogs of WFA such as those described in US 2017/0066797 and U.S. Pat. No. 9,084,800 incorporated herein by reference.

The compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof.

The compounds of the present disclosure are useful for treating cachexia and/or a disease or disorder that induces cachexia. Exemplary disorders include but are not limited to cancers, neurological disorders, HIV infection, AIDS, sepsis, chronic pulmonary diseases such as COPD, and cardiac disorders such as heart failure. Neurological disorders typically associated with cachexia include but are not limited to familial amyloid polyneuropathy, multiple sclerosis, and Riley-Schwachman syndrome. Examples of pulmonary diseases associated with cachexia include but are not limited to chronic obstructive pulmonary disease, pneumonia, abscess of the lung, and cystic fibrosis. Examples of cardiac diseases associated with cachexia include but are not limited to congestive heart failure, endocarditis, and polyarteritis nodosa. Examples of oncologic conditions associated with cachexia include but are not limited to lung cancer, colon cancer, breast cancer, pancreatic cancer, leukemia, Kaposi sarcoma, liver cancer, stomach cancer, and paraneoplastic syndrome.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness.

The term “cancer metastasis” has its general meaning in the art and refers to the spread of a tumor from one organ or part to another non-adjacent organ or part.

Any cancer or metastatic cancer that induces cachexia may be targeted using the inventive therapy including, but not limited to, ovarian cancer, breast cancer, prostate cancer, endometrial cancer, uterine cancer, renal cancer, liver cancer, melanoma, pituitary cancer, testicular cancer, non-Hodgkin's lymphoma, pancreatic cancer, colon cancer, lymphoma, myeloma, brain cancer, kidney cancer, lung cancer, spleen cancer, gall bladder cancer, anal cancer, cervical cancer, and bone cancer.

The terms “subject” and “patient” are used interchangeably herein, and refer to an animal such as a mammal, which is afflicted with or suspected of having, at risk of, or being pre-disposed to cancer. The terms may refer to a human. The terms also include domestic animals bred for food, sport, or as pets, including horses, cows, sheep, poultry, fish, pigs, cats, dogs, and zoo animals, goats, apes (e.g. gorilla or chimpanzee), and rodents such as rats and mice. Typical subjects include persons susceptible to, suffering from or that have suffered from cancer.

The term “treating” or “treatment”, as used herein, means reversing, alleviating, inhibiting the progress of, or ameliorating the disorder or condition to which such term applies, or one or more symptoms of such disorder or condition. For example, the treatment of the disclosure may slow or inhibit the progress of cachexia or eliminate the cachexia, e.g. by reducing myofibrillar atrophy and/or conversion of type IIA myofibers to type IIB myofibers, slow the growth of the cancer, reduce the number of tumor cells in said cancer, reduce tumor load, eliminate said cancer, preserve systolic function, improve diastolic function, or eliminate arrhythmia.

By a “therapeutically effective amount” is meant a sufficient amount of the molecule to treat cachexia or a disorder that induces cachexia at a reasonable benefit/risk ratio applicable to any medical treatment. It will be understood, however, that the total daily usage of the molecules and compositions of the present disclosure will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific polypeptide employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

Any method of administration may be used to deliver the compound of the disclosure to the subject. In particular embodiments, the route of administration may be oral, intravenous, intraarterial, intraperitoneal, or subcutaneous, for example. In certain embodiments, the compound of the disclosure may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. Multiple administrations may be by the same route or by different routes. In some embodiments, multiple doses, e.g. 2, 3, 4, 5, or more doses are given over a period of time, e.g. over 2, 3, 4, 5, 6, 7, 8, 9, 10, or more days.

Another aspect of the disclosure relates to a pharmaceutical composition comprising a compound according to the disclosure and a pharmaceutically acceptable carrier. “Pharmaceutically” or “pharmaceutically acceptable” refer to molecular entities and compositions that do not produce an adverse, allergic or other untoward reaction when administered to a subject, such as a human, as appropriate. A pharmaceutically acceptable carrier or excipient refers to a non-toxic solid, semi-solid or liquid filler, diluent, encapsulating material or formulation auxiliary of any type.

Solutions comprising compounds of the disclosure as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming micro-encapsule matrices of the drug in biodegradable polymers such as poly(lactide-co-glycolide). Depending upon the ratio of drug to polymer and the nature of the particular polymer employed, the rate of drug release can be controlled.

Examples of other biodegradable polymers include poly(ortho-esters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner.

Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the compounds described herein are administered without any other active agent. In some embodiments, the compounds described herein may be combined with standard-of-care treatments (e.g., radiation therapy, hormonal therapy). In some embodiments, the compound of the disclosure may be administered sequentially or concomitantly with one or more chemotherapeutic or radiotherapeutic agents.

In one embodiment, said chemotherapeutic or radiotherapeutic agents are a therapeutic active agent used as an anticancer agent. For example, said anticancer agents include but are not limited to fludarabine, gemcitabine, capecitabine, methotrexate, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbazine, epipodophyllotoxins such as etoposide and teniposide, camptothecins such as irinotecan and topotecan, bleomycin, doxorubicin, idarubicin, daunorubicin, dactinomycin, plicamycin, mitoxantrone, L-asparaginase, doxorubicin, epirubicin, 5-fluorouracil and 5-fluorouracil combined with leucovorin, taxanes such as docetaxel and paclitaxel, levamisole, estramustine, nitrogen mustards, nitrosoureas such as carmustine and lomustine, vinca alkaloids such as vinblastine, vincristine, vindesine and vinorelbine, imatinib mesylate, hexamethylmelamine, kinase inhibitors, phosphatase inhibitors, ATPase inhibitors, tyrphostins, protease inhibitors, inhibitors herbimycin A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxins, hormonal therapies, retinoids, photosensitizers or photodynamic therapies, angiogenesis inhibitors, antimitotic agents, isoprenylation inhibitors, cell cycle inhibitors, actinomycin, bleomycin, anthracyclines, MDR inhibitors and Ca²⁺ ATPase inhibitors.

Additional anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Further therapeutic active agents may be an antiemetic agent. Suitable antiemetic agents include, but are not limited to, metoclopramide, domperidone, prochlorperazine, promethazine, chlorpromazine, trimethobenzamide, ondansetron, granisetron, hydroxyzine, acetylleucine, alizapride, azasetron, benzquinamide, bietanautine, bromopride, buclizine, clebopride, cyclizine, dimenhydrinate, diphenidol, dolasetron, meclizine, methallatal, metopimazine, nabilone, pipamazine, scopolamine, sulpiride, tetrahydrocannabinols, thiethylperazine, thioproperazine and tropisetron. In a preferred embodiment, the antiemetic agent is granisetron or ondansetron.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, buprenorphine, meperidine, loperamide, ethoheptazine, betaprodine, diphenoxylate, fentanyl, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflunisal, etodolac, fenoprofen, flurbiprofen, ibuprofen, ketoprofen, indomethacin, ketorolac, meclofenamate, mefenamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, clorazepate, clonazepam, chlordiazepoxide and alprazolam.

The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention. Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

### EXAMPLES

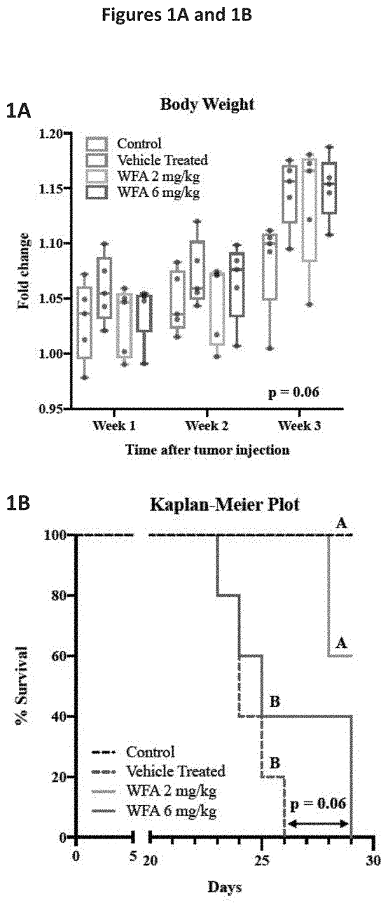

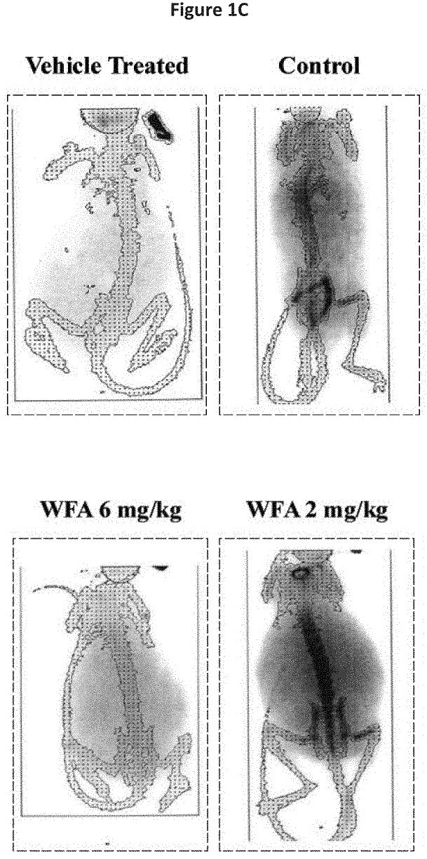

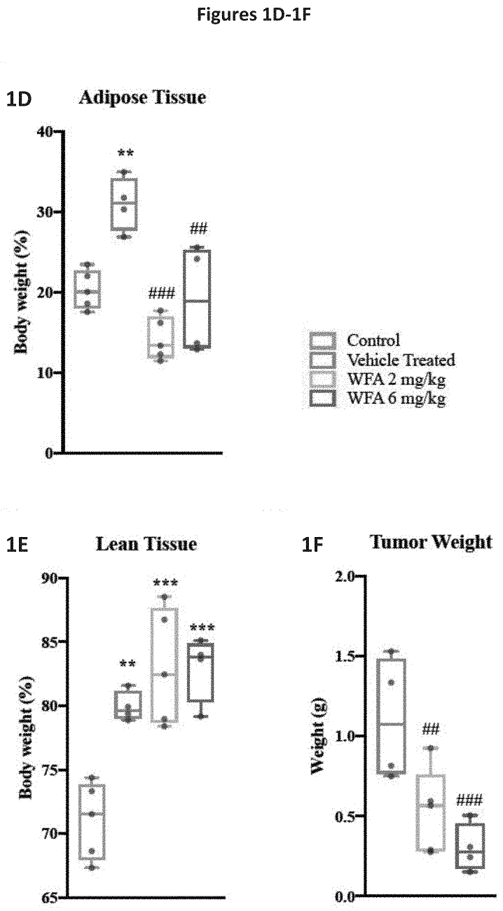

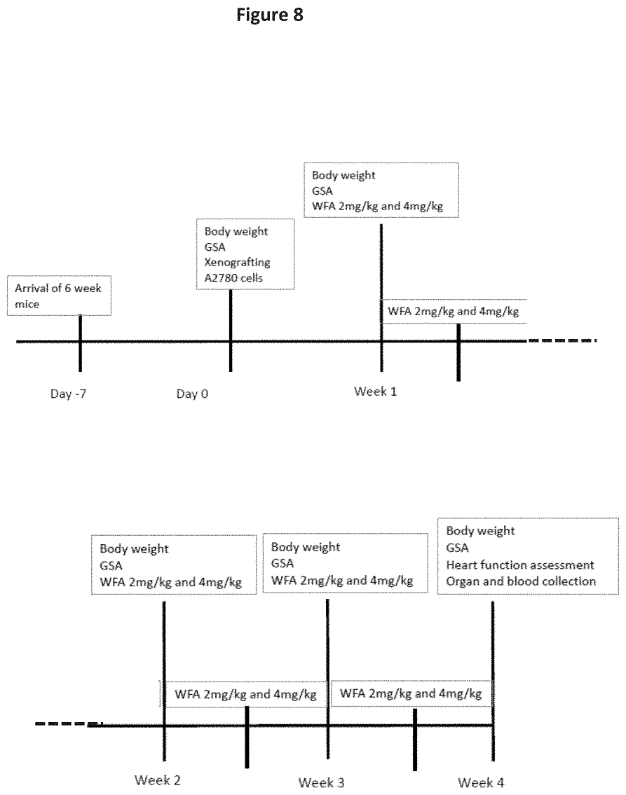

The following Examples provide exemplary designs and methods for practicing the invention. These Examples describe materials and methods for using embodiments illustrated in FIGS. 1-25. Additional details about the drawings can be found in the section entitled “Brief Description of the Drawings”.

In the following Examples, the steroidal lactone WFA ameliorated body changes associated with ovarian cancer and the subsequent cachectic phenotype. Mice treated with WFA following xenografting of the A2780 ovarian cancer line exhibited a significant increase in survival rate, a restoration of grip strength, and an improvement in myofibrillar cross-sectional area. Further, treatment completely abolished the slow-to-fast myofiber type conversion observed in the settings of cancer cachexia. The Examples show that treatment with WFA led to a reduction of NF-κB-related proinflammatory cytokines and NLRP3 inflammasome signaling. Cumulatively, the Examples demonstrate a previously overlooked role for WFA in the context of ovarian cancer and cancer cachexia. Based on this information, the use of female mice as a model of ovarian cancer provides a valid model to examine the induction of cachexia by ovarian cancer.

### Example 1

Materials and Methods

Cell Line: The A2780 ovarian epithelial cancer cell line was maintained in Roswell Park Memorial Institute (RPMI) medium-1640 supplemented with: 10% fetal bovine serum (FBS, Hyclone), 100 U/ml penicillin, and 10 μg/ml streptomycin. Cells were cultured in a humidified atmosphere of 5% CO₂ at 37° C., and the medium was changed every 48 hours.

Generation of Tumor in Mice: Six-week old female NOD.Cg-Prkdc^{scid} Il2rg^{tm1Wjl}/SzJ (NSG, Jackson Lab Strain #005557) immunodeficient mice were randomly assigned to one of three tumor-bearing groups or a control group (5 animals/group). Tumor-bearing groups received an IP injection of 1.0×10⁶ low passage A2780 cells suspended in 100 μl sterile PBS. Control group received IP injection of 100 μl sterile PBS alone. After an initial refractory period of eight days, the mice received an IP injection of vehicle (10% dimethyl sulfoxide, 90% glycerol trioctanoate) or WFA once every three days. The desired endpoint was 5 weeks post-tumor implantation. However, the mice were euthanized 29 days post-implantation due to several mice reaching the criteria for an early humane endpoint. Post-euthanization, several tissues were collected, weighed, snap frozen in liquid nitrogen and then stored at −80° C. for further analysis or fixed in buffered formalin solution. Mice were housed in a 12-hour light-dark cycle and given water and food ad libitum. The Institutional Animal Care and Use Committee (IACUC, protocol #15405) and Institutional Biosafety Committee (IBC, protocol #18-208) of the University of Louisville approved all experimental protocols in mice in advance.

Grip Strength Measurements: Before assessment, mice were weighed on a commercially available digital scale. Forepaw and total grip strength of mice were measured using a digital grip strength meter (Columbus Instruments, Columbus, Ohio, USA) and then normalized by total body weight. Before the beginning of the test, the mice were acclimatized for five minutes. The mouse was allowed to grasp the total paw pull-bar assembly, and in a separate experiment the forepaw pull-bar assembly. The mouse was then gently drawn with constant force in a straight line away from the device until the mouse could no longer grasp the bar. Force at time of release was recorded as the peak tension. Each mouse was tested five times with a delay of 20-40 seconds between each testing. The mean peak tension was calculated from the recordings normalized by total body weight.

Analysis of Body Composition: Body fat and lean mass composition, as well as the bone mineral content and density, excluding head region of interest (ROI), were determined by dual-energy X-ray absorptiometry (DEXA) scan using a mouse densitometer (PIXImus 2; Lunar, Madison, Wis., USA). According to the manufacturer's guidelines to calibrate and validate the performance of the apparatus, a “mouse phantom” provided with the machine was scanned before scanning the first mouse. Animals were euthanized immediately before assessment by DEXA scan. The mouse was then transferred to the disposable PIXImus measuring tray in the prone position with their head in a loosely fitting nose cone to aid positioning. Upon completion of the DEXA scan, approximately five minutes, the mouse was placed on ice to preserve tissue prior to collection.

Histology and Morphometric Analysis of Skeletal Muscle: The tibialis anterior (TA), gastrocnemius (GA) and quadriceps femoris (QF) of the mice were isolated, flash frozen in liquid nitrogen, mounted in OCT embedding medium, and then sectioned using a microtome cryostat. To assess tissue morphology, 10 μm thick transverse sections were cut from the mid-belly of the muscle and then subjected to hematoxylin and eosin (H&E) staining. Images of H&E-stained TA/GA/QF muscle sections were quantified using Image J and/or Fiji software to measure myofiber cross-sectional area (CSA). Myofiber CSA was calculated by analyzing ˜350-500 myofibers per muscle.

Total RNA Extraction and qPCR: Isolation of total RNA from xenografted tumor samples was performed using an RNeasy® Mini Kit (Qiagen Catalog #74104) according to the manufacturer's instructions. Isolation of total RNA from skeletal muscle was performed using an RNeasy® Fibrous Tissue Mini Kit (Qiagen Catalog #74704) according to the manufacturer's instructions. First strand cDNA was synthesized using 1 μg of purified RNA and a commercially available kit (iScript™ cDNA synthesis, Bio-Rad Catalog #170-8891), Quantification of mRNA expression was performed similar to as previously described (Hindi and Kumar, 2016) using the SYBR Green dye method on a StepOnePlus™ system (Applied Biosystems) using gene-specific primers, as shown in Table 1.

TABLE 1
Human and mouse gene specific primer sequences.

| Gene | Species | Direction | Nucleotide Sequence Identity | SEQ ID NO |
|---|---|---|---|---|
| TNFα | Homo | Forward | 5′-CCCAGGGACCTCTCTCTAATC-3′ | SEQ ID NO: 01 |
| | sapiens | Reverse | 5′-ATGGGCTACAGGCTTGTCACT-3′ | SEQ ID NO: 02 |
| TNFα | Mus | Forward | 5′-AGCACAGAAAGCATGATCCG-3′ | SEQ ID NO: 03 |
| | musculus | Reverse | 5′-GCCACAAGCAGGAATGAGAA-3′ | SEQ ID NO: 04 |
| IFNγ | Homo | Forward | 5′-CTAATTATTCGGTAACTGACTTGA-3′ | SEQ ID NO: 05 |
| | sapiens | Reverse | 5′-ACAGTTCAGCCATCACTTGGA-3′ | SEQ ID NO: 06 |
| IFNγ | Mus | Forward | 5′-GACAATCAGGCCATCAGCAAC-3′ | SEQ ID NO: 07 |
| | musculus | Reverse | 5′-CGGATGAGCTCATTGAATGCTT-3′ | SEQ ID NO: 08 |
| IL-6 | Homo | Forward | 5′-ACACAGACAGCCACTCACCT-3′ | SEQ ID NO: 09 |
| | sapiens | Reverse | 5′-TTCTGCCAGTGCCTCTTTGC-3′ | SEQ ID NO: 10 |
| IL-6 | Mus | Forward | 5′-CCTTCTTGGGACTGATGCTGG-3′ | SEQ ID NO: 11 |
| | musculus | Reverse | 5′-GCCTCCGACTTGTGAAGTGGT-3′ | SEQ ID NO: 12 |
| IL-8 | Homo | Forward | 5′-AAACCACCGGAAGGAACCAT-3′ | SEQ ID NO: 13 |
| | sapiens | Reverse | 5′-CCTTCACACAGAGCTGCAGAAA-3′ | SEQ ID NO: 14 |
| MIP-2 | Mus | Forward | 5′-CCACTCTCAAGGGCGGTCAAA-3′ | SEQ ID NO: 15 |
| | musculus | Reverse | 5′-TACGATCCAGGCTTCCCGGGT-3′ | SEQ ID NO: 16 |
| NLRP3 | Homo | Forward | 5′-GTGTGGGACTGAAGCACCTG-3′ | SEQ ID NO: 17 |
| | sapiens | Reverse | 5′-GTCTCCCAAGGCATTCTCCC-3′ | SEQ ID NO: 18 |
| NLRP3 | Mus | Forward | 5′-AGAAGAGACCACGGCAGAAG-3′ | SEQ ID NO: 19 |
| | musculus | Reverse | 5′-CCTTGGACCAGGTTCAGTGT-3′ | SEQ ID NO: 20 |
| CASP1 | Homo | Forward | 5′-GCCTGTTCCTGTGATGTGGAG-3′ | SEQ ID NO: 21 |
| | sapiens | Reverse | 5-TGCCCACAGACATTCATACAGTTTC-3′ | SEQ ID NO: 22 |
| CASP1 | Mus | Forward | 5′-CACAGCTCTGGAGATGGTGA-3′ | SEQ ID NO: 23 |
| | musculus | Reverse | 5′-GGTCCCACATATTCCCTCCT-3′ | SEQ ID NO: 24 |
| IL-1β | Homo | Forward | 5′-ACAGATGAAGTGCTCCTTCCA-3′ | SEQ ID NO: 25 |
| | sapiens | Reverse | 5′-GTCGGAGATTTCGTACTGGAT-3′ | SEQ ID NO: 26 |
| IL-1β | Mus | Forward | 5′-TCACAGCAGCACATCAACAA-3′ | SEQ ID NO: 27 |
| | musculus | Reverse | 5′-TGTCCTCATCCTGGAAGGTC-3′ | SEQ ID NO: 28 |
| IL-1β | Homo | Forward | 5′-CGGCCTCTATTTGAAGATATGAC-3′ | SEQ ID NO: 29 |
| | sapiens | Reverse | 5′-CCATACCTCTAGGCTGGCTA-3′ | SEQ ID NO: 30 |
| IL-1β | Mus | Forward | 5′-ACAACTTTGGCCGACTTCAC-3′ | SEQ ID NO: 31 |
| | musculus | Reverse | 5′-GGGTTCACTGGCACTTTGAT-3′ | SEQ ID NO: 32 |
| HO-1 | Homo | Forward | 5′-GGGTGATAGAAGAGGCCAAGACT-3′ | SEQ ID NO: 33 |
| | sapiens | Reverse | 5′-AGCTCCTGCAACTCCTCAAGA-3′ | SEQ ID NO: 34 |
| HO-1 | Mus | Forward | 5′-TGAAGGAGGCCACCAAGGAGG-3′ | SEQ ID NO: 35 |
| | musculus | Reverse | 5′-AGAGGTCACCCAGGTAGCGGG-3′ | SEQ ID NO: 36 |
| GAPDH | Homo | Forward | 5′-TGATGACATCAAGAAGGTGGT-3′ | SEQ ID NO: 37 |
| | sapiens | Reverse | 5′-TCCTTGGAGGCCATGTGGGCC-3′ | SEQ ID NO: 38 |
| β-Actin | Mus | Forward | 5′-CAGGCATTGCTGACAGGATG-3′ | SEQ ID NO: 39 |
| | musculus | Reverse | 5′-TGCTGATCCACATCTGCTGG-3′ | SEQ ID NO: 40 |

Skeletal Muscle Fiber-type Immunostaining: To determine the composition of different types and gross composition of skeletal muscle fibers, 10 μm thick transverse sections were made from TA muscles, a hydrophobic boundary was drawn around the section, and then blocked with 5% goat serum and 2% bovine serum albumin (BSA) in PBS for 30 minutes. The sections were then incubated for one hour with monoclonal antibodies against myosin heavy chain (MyHC) isoforms type I, IIa, and IIb using clone BA-D5, SC-7, and BF-F3, respectively (Developmental Studies Hybridoma Bank, Iowa City, Iowa, USA). The following secondary antibodies were then used for detection after 1-2 hours of incubation period: anti-Mouse IgG2b (γ2b) CF™ 350 antibody produced in goat, anti-Mouse IgG1 (yl) CF™ 568 antibody produced in goat, and Alexa Fluor-488 goat anti-mouse IgM. Fluorescence images were captured with a Nikon TiE 3000 inverted microscope, the single-color channels were merged, and percentage of each fiber-type in whole muscle section was recorded. Nonspecific background staining was performed using the above protocol but omitting the inclusion of the primary antibodies. Antibodies BA-D5, SC-71, and BF-F3 were deposited to the DSHB by Schiaffino, S. (DSHB Hybridoma Product BA-D5, SC-71, and BF-F3). Table 2 discloses a complete list of antibodies utilized.

TABLE 2
Primary and secondary antibody list.

| Antibody | Company | Catalog # | Application | Dilution |
|---|---|---|---|---|
| Monoclonal anti-MyHC | Developmental Studies | BA-D5 | IHC | 1:100 |
| Type I | Hybridoma Bank | | | |
| Monoclonal anti-MyHC | Developmental Studies | SC-71 | IHC | 1:100 |
| Type IIa | Hybridoma Bank | | | |
| Monoclonal anti-MyHC | Developmental Studies | BF-F3 | IHC | 1:100 |
| Type IIb | Hybridoma Bank | | | |
| Polyclonal anti-phospho- | Sigma-Aldrich | SAB4300009 | IHC | 1:50 |
| RelA (pSer⁵³⁶) | | | WB | 1:1000 |
| Polyclonal anti-RelA | Sigma-Aldrich | SAB4300295 | WB | 1:1000 |
| Monoclonal anti-β-Actin | Sigma-Aldrich | A3854 | WB | 1:2000 |
| (HRP Linked) | | | | |
| Monoclonal anti-IL-1 | R&D Systems | MAB601 | IHC | 1:100 |
| beta | | | | |
| (Mouse IgG1) | | | | |
| Monoclonal anti-NLRP3 | R&D Systems | MAB7578 | IHC | 1:100 |
| (Rat IgG2a) | | | | |
| Polyclonal anti-IL-18 | R&D Systems | AF2548 | IHC | 1:100 |
| (Goat IgG) | | | | |
| Polyclonal anti-Caspasel | Sigma-Aldrich | AB1871 | IHC | 1:100 |
| (Rabbit IgG) | | | | |
| Polyclonal anti-HO-1 | Enzo Scientific | ADI-SPA-895-D | IHC | 1:100 |
| (Rabbit) | | | | |
| Alexa Fluor ™ 488 Goat | Thermo Fisher | A-21042 | IHC | 1:2000 |
| anti-Mouse IgM (μ) | Scientific | | | |
| CF ™ 568 Goat anti- | Sigma-Aldrich | SAB4600313 | IHC | 1:2000 |
| Mouse IgG1 (γ1) | | | | |
| CF ™ 350 Goat anti- | Sigma-Aldrich | SAB4600228 | IHC | 1:2000 |
| Mouse IgG2b (γ2b) | | | | |
| Alexa Fluor ™ 594 Goat | Thermo Fisher | A-11012 | IHC | 1:2000 |
| anti-Rabbit IgG (H + L) | Scientific | | | |
| Alexa Fluor ™ 488 | Thermo Fisher | A-21210 | IHC | 1:2000 |
| Rabbit anti-Rat IgG | Scientific | | | |
| (H + L) | | | | |
| Alexa Fluor ™ 532 Goat | Thermo Fisher | A-11009 | IHC | 1:2000 |
| anti-Rabbit IgG (H + L) | Scientific | | | |
| CF ™ 594 Goat anti- | Sigma-Aldrich | SAB4600326 | IHC | 1:2000 |
| Mouse IgG1 | | | | |
| CF ™ 488 Bovine anti- | Biotium | 20293-1 | IHC | 1:2000 |
| Goat IgG (H + L) | | | | |
| ECL Donkey anti-rabbit | Millipore Sigma | GENA934 | WB | 1:3000 |
| HRP Linked Whole Ab | | | | |

WB = Western Blot; IHC = Immunohistochemistry.

Immunohistochemistry of Tumor Tissue: For the detection of various proteins, 10 μm thick sections were made from the midsection of tumor samples. A hydrophobic boundary was drawn around the sections and then they were fixed in 3.7% formaldehyde solution for 10 minutes. The slides were washed three times with 1×PBS for 5 minutes each. The sections were then blocked in 2% BSA in PBS for 30 minutes. The sections were then incubated with primary antibodies (1:100 dilution) overnight in a humidity chamber at 4° C. The sections were then washed three times with 1×PBS for 5 minutes. The slides were incubated in appropriate secondary antibodies (1:2000 dilution) for 45-60 minutes. Sections were counterstained with DAPI (1:2500 dilution) for 5 minutes to visualize nuclei. Slides were then washed twice in 1×PBS for 5 minutes, and then mounted for visualization. Nonspecific background staining was determined by performing the above protocol with omitting the inclusion of the primary antibodies. Refer to Table 2 for a complete list of antibodies utilized.

Imaging: Slides were mounted using Eukitt® quick-hardening mounting medium (Sigma) and visualized at −0.4° C. on a Nikon TiE 3000 inverted microscope (Nikon) equipped with a digital camera (DS-U2/L2-Ri1 digital microscope camera (Nikon) for light microscopy or DXM-1200C coded digital camera (Nikon) for fluorescent microscopy), and Nikon NIS Elements AR software (Nikon). Exposure times were consistent for each staining type. Image levels were equally adjusted using Adobe Photoshop CS6 software (Adobe) to remove nonspecific background staining.

Protein Extraction and Western Blotting: Tumor samples were homogenized in chilled RIPA buffer (Sigma) supplemented with a Complete Mini Protease Inhibitor tablet (Roche Molecular Biochemicals, Indianapolis, Ind.). Tissue lysates were centrifuged at 10,000 RPM and the supernatants were collected. Protein concentration for each sample was determined using the Bradford reagent method (Bio-Rad), according to manufacturer's instructions. Protein lysates (50 bg) were separated on 10% SDS-PAGE gels at 100 volts for 2 h. The proteins were transferred to nitrocellulose membranes at 100 volts for 90 minutes. The membranes were blocked with 5% non-fat milk in Tris-buffered saline supplemented with Tween®20 (0.5%; TBS-T) for 30-60 minutes. The membranes were then washed 3 times with TBS-T for 5 minutes. The membranes were then incubated with primary antibody at 4° C. overnight. The membranes were washed 3 times with TBS-T for 5 minutes each followed by incubation in TBS-T containing horseradish peroxidase conjugated secondary antibody (1:2000 dilution) for 1 h. The membranes were rinsed 3 times with TBS-T for 5 minutes each. Visualization of immunoreactive bands was enhanced using chemiluminescence reagents (Sigma). The membranes were stripped off using Restore™ Western Blot Stripping Buffer (Thermo Scientific Catalog #21059) for 30 minutes and re-probed with horseradish peroxidase-conjugated β-Actin used as a control to normalize the loading variation. Refer to Table 2 for a complete list of antibodies utilized.

Human MAP Multiplex Assay: Milliplex® MAP Human 11-18 Singleplex Magnetic Bead Kit (Catalog #HIL-18MAG-55K, Lot #3179880) was reconstituted per the manufacturer's protocol and utilized to reconstitute the Milliplex® MAP Human Cytokine/Chemokine Magnetic Bead Panel (Catalog #HCYTOMAG-60K, Lot #3203050, 9 Analytes) according to manufacturer's instructions. Reconstituted MAP multiplex was performed according to manufacturer's protocols with the exception of the following modifications. Assay buffer LE-ABGLP2 was used instead of the one provided with the kit to lyse tumor samples/extract protein at the recommendation of the manufacturer. Briefly, tumor samples were bisected over dry ice and then transferred into a 1.5 ml micro-centrifuge tube containing 250 μl assay buffer LE-ABGLP2 per 100 mg tissue. Samples were minced on ice with sterilized scissors. Minced tissue was subjected to sonication for 30 seconds on ice. Gross cellular debris was removed by centrifugation at 10 k RPM for 3 minutes at room temperature. Four blanks were employed instead of two and an additional dilution was included in the standard curve (0.64 pg/ml). Two concentrations of the samples (10 and 50 μg total protein) were run in triplicate. Assay buffer LE-ABGLP2 was used for the blank wells and to dilute samples. Graphical Display and Statistical Analyses: The majority of the results were expressed as box-and-whisker plots with the box comprised of the first, second, and third quartiles, and the lower and upper whiskers corresponding to the minimum and maximum values, respectively, to display the entire range of data. A Kaplan-Meier curve was used for the survival analysis. Statistical analysis of the data was performed using one-way analysis of variance (ANOVA) followed by Tukey's Honestly Significant Difference Test (HSDT) post hoc analysis or the Mantel-Cox log-rank test to determine statistically significant differences between groups with GraphPad Prism 8.0.1 software for Mac (La Jolla, Calif., USA). A value of P<0.05 was considered statistically significant, unless otherwise specified.

Results

WFA Impedes the Increase in Mortality and Body Composition Changes Associated with Ovarian Cancer.

To assess the effects of WFA on ovarian cancer-induced cachexia, female severely immunodeficient NSG (Jackson Lab) mice received intraperitoneal (IP) injections of A2780 cells to generate a xenograft model of ovarian cancer or sterile saline to assess the normal development of mice, henceforth referred to as control group. After an initial lag phase, one group of tumor-bearing mice were treated with one of two doses of WFA (2 mg/kg or 6 mg/kg), henceforth referred to as WFA-treated. A second group of tumor-bearing mice were injected with vehicle, henceforth referred to as vehicle treated group. The control group also received injections of vehicle. We recorded a baseline body weight from all mice one day after IP injection of A2780 cells, and subsequently every week until the end of the study, as shown in Table 3.

TABLE 3
Quantitative assessment of body and organ weight.

| Group | Control | Vehicle Treated | WFA 2 mg/kg | WFA 6 mg/kg |
|---|---|---|---|---|
| Initial BW (g) | 18.71 ± 0.91 | 18.96 ± 2.11 | 19.14 ± 1.50 | 19.60 ± 0.76 |
| BW Week 1 (g) | 19.51 ± 1.72 | 20.46 ± 1.80 | 19.98 ± 2.33 | 20.70 ± 1.35 |
| BW Week 2 (g) | 19.94 ± 1.50 | 20.82 ± 1.85 | 20.43 ± 2.43 | 21.37 ±1.32 |
| BW Week 3 (g) | 20.83 ± 1.53 | 22.65 ± 2.00 | 22.68 ± 2.53 | 23.57 ± 1.23 |
| Kidney (mg/g) | 6.26 ± 0.57 | 6.08 ± 1.03 | 4.95 ± 0.79 | 5.45 ± 0.49 |
| Liver (mg/g) | 53.67 ± 4.65 | 44.54 ± 3.64 | 47.16 ± 15.88 | 41.18 ± 3.60 |
| Spleen (mg/g) | 1.41 ± 0.53 | 2.06 ± 0.11 | 2.20 ± 1.74 | 2.78 ± 1.45 |

No statistically significant difference between the groups, as determined by one-way analysis of variance (ANOVA), was found by the termination of the study in body weight fold change (FIG. 1A). However, there was a trend towards an increase (F(3, 16)=2.874 with an associated p-value of 0.06) in the body weight change of the tumor bearing groups at the endpoint, which is due to an increase in tumor weight. This experiment has subsequently been further refined in Example 3. The study was intended to last for 5 weeks post-tumor implantation, but shortly after our assessment at week 3, mice had begun to succumb to tumor burden or met the criteria for an early humane endpoint. A survival analysis was performed amongst the four groups to elucidate whether treatment had any effect on mortality (FIG. 1B). To analyze the Kaplan-Meier plot, the Mantel-Cox log-rank test was performed and an associated p-value of 0.0003 was determined (χ²=19.17, df=3). There was no significant difference between the control group and the group of mice treated with 2 mg/kg of WFA, but these groups showed a higher survival rate than the WFA 6 mg/kg and vehicle treated groups. No significant difference between the 6 mg/kg WFA and vehicle treated groups was found, although there was a trend towards an increase in survival (p=0.06) in the WFA 6 mg/kg group. We assessed the normalized weight of select vital organs due to the differences in survival, as shown in Table 1. Despite evidence of several tumors found throughout the peritoneal cavity, we found no statistically significant changes in our assessment of select vital organs.

In addition to an increased rate of mortality, cachexia generally results in the wasting of skeletal muscle. In order to determine whether ovarian cancer or WFA had any effect on body composition, carcasses were assessed by dual-energy X-ray absorptiometry (DEXA) scan. FIG. 1C shows representative DEXA scan images from control, vehicle treated, WFA 2 mg/kg, and WFA 6 mg/kg groups. As determined by one-way ANOVA and Tukey's Honestly Significant Difference Test (HSDT), there was a statistically significant increase in the proportion of adipose tissue to whole body weight in the vehicle treated group compared to the control group (F(3, 14)=13.67; p=0.006) (data shown in FIG. 1D), consistent with published reports linking a generalized accumulation of adipose tissue in the thoracoabdominal region with ovarian cancer. There was an attenuation of adipose tissue following treatment of WFA at both concentrations compared to the vehicle treated group (WFA 2 mg/kg: p=0.0001; WFA 6 mg/kg: 0.0040) (FIG. 1D). Paradoxically, there was a surprising and significant increase in the proportion of lean tissue to body weight in all tumor-bearing groups regardless of treatment with WFA (F(3, 14)=15.13; vehicle treated: p=0.0048; WFA 2 mg/kg: p=0.0002; WFA 6 mg/kg: p=0.0004), contrary to what one would expect in a cachectic state, as shown in FIG. 1E. Upon examination of the DEXA scan images, it appears that the solid tumors might have the same radiographic signature as the lean tissue, potentially explaining the paradoxical increase in lean tissue. No significant differences were found in bone mineral density or content.

Free peritoneal tumors (visible tumors) not associated with an organ were collected and weighed to assess WFA effects. FIG. 1F shows that WFA significantly reduced the size of peritoneal tumors as compared to the vehicle treated group (F(2, 10)=8.746; WFA 2 mg/kg: p=0.03; WFA 6 mg/kg: p=0.0061), but no significant difference was observed between the two doses of WFA (p=0.48). At the time of euthanization and tissue collection, tumors associated with the intestines were found most frequently in the vehicle treated group and were of the largest size. In contrast, WFA treatment resulted in fewer and smaller tumors, with the 6 mg/kg group having the smallest tumors. Cumulatively, these data indicate that treatment with WFA improves the survival of mice xenografted with A2780 cells and rescues some of the body changes associated with ovarian cancer. The DEXA scan data in conjunction with the collection and assessment of intraperitoneal tumors suggests attenuation in the loss of lean tissue, and therefore skeletal muscle. Without being bound by theory, this may be due to the radiographic abnormalities exhibited by the xenografted tumors.

WFA Treatment Partially Rescues the Gonadal Fat Pad.

Ovarian cancer progression is often associated with the specific loss of the gonadal fat pad. Thus, an attempt was made to quantify the amount of adipose tissue present in the gonadal region at the time of tissue collection post-euthanization. The severe reduction in the gonadal fat pad rendered accurate measurement of gonadal adipose tissue improbable. Further, in some mice, there was a complete absence of gonadal fat. Alternatively, the mice in each group were qualitatively scored based on of the degree of gonadal fat present, as shown in Table 4, as follows:

1. When the ovaries were embedded in adipose tissue and not readily identifiable without dissection through the fat pad, the mouse was scored “Abundant”. All mice in the control group were deemed to have an abundant amount of gonadal fat, whereas the vehicle treated group had no gonadal fat present, consistent with published reports linking specific changes in the gonadal fat pad to the progression of ovarian cancer.

2. When adipose tissue was present but in such a scant quantity that we could not accurately quantify the mass, the amount of gonadal fat was marked as “Small Amount”.

3. When adipose tissue was present but did not meet the criteria for “Abundant” or “Small Amount”, the mouse was scored as “Present”.

TABLE 4
Qualitative assessment of gonadal fat.

| | Gonadal Fat | | | |
|---|---|---|---|---|
| Group | Absent | Small Amount | Present | Abundant |
| Control | 0 | 0 | 0 | 5/5 |
| Vehicle-treated | 4/4 | 0 | 0 | 0 |
| WFA 2 mg/kg | 0 | 1/5 | 4/5 | 0 |
| WFA 6 mg/kg | 0 | 3/4 | 1/4 | 0 |

There was a reduction in the gonadal fat pad in both WFA 2 mg/kg and 6 mg/kg groups, but they were not completely abolished. While qualitative, this data is further indicative that treatment with WFA impedes but does not completely resolve body composition changes caused by ovarian cancer.

WFA Treatment Functionally Rescues Muscle Strength in the Settings of Ovarian Cancer-Induced Cachexia.

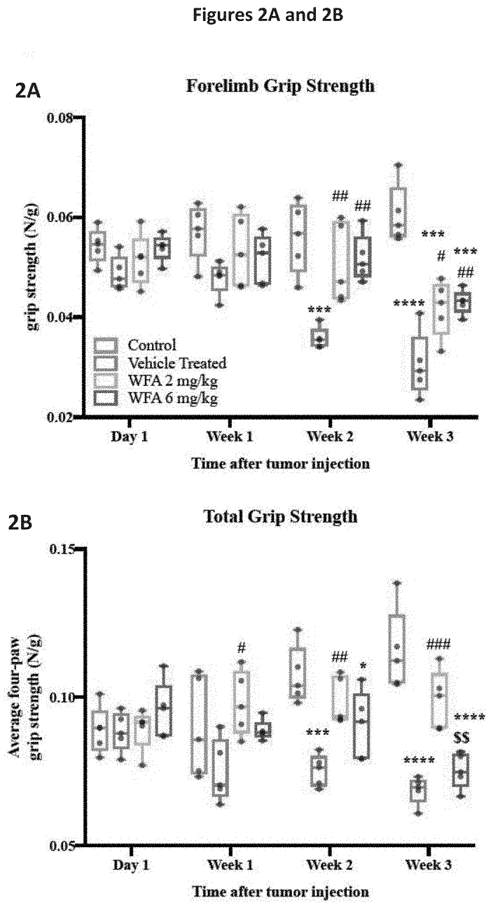

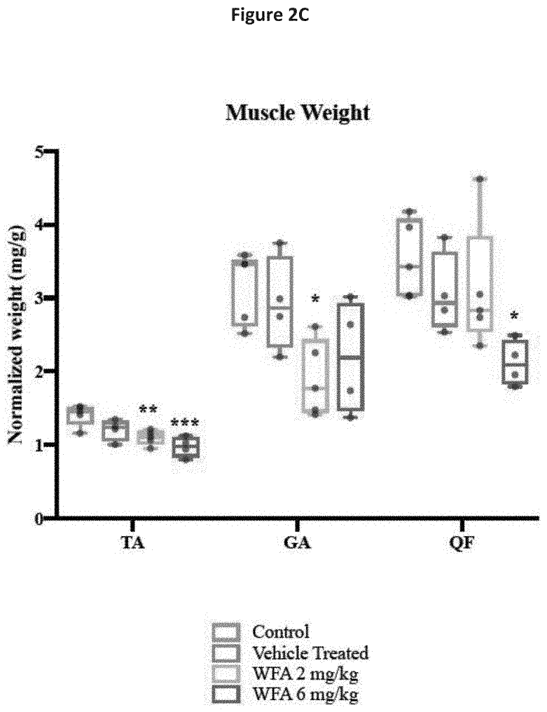

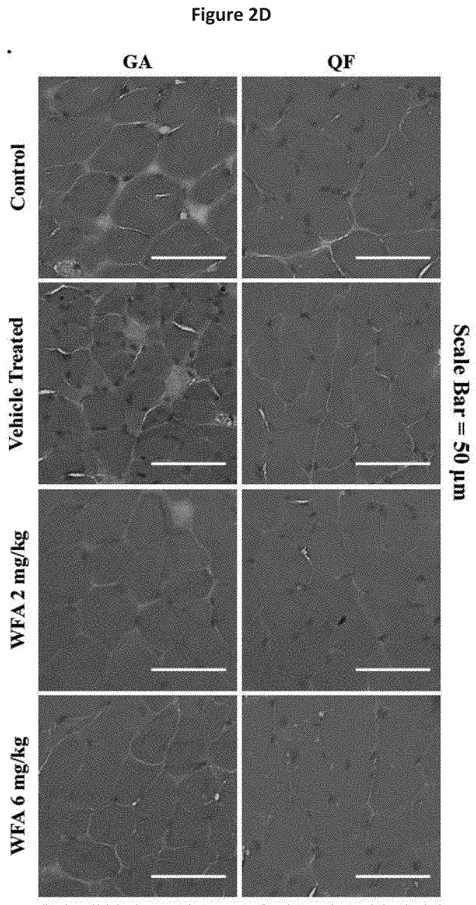

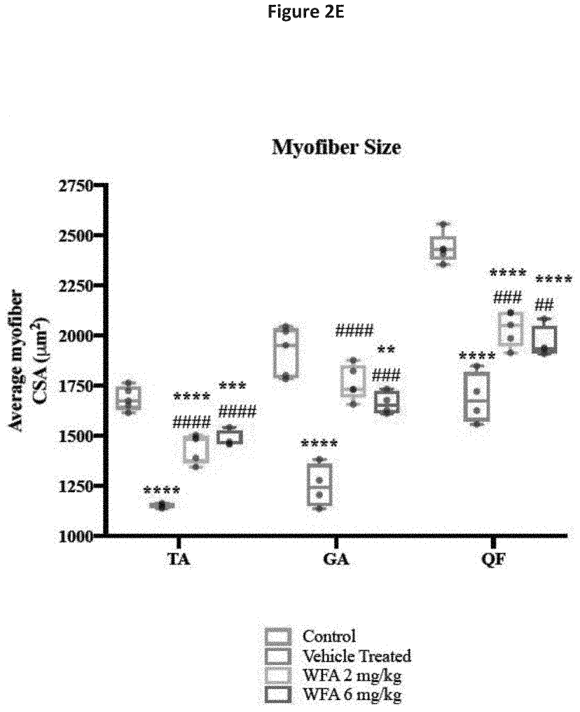

A major hallmark of cachexia is a reduction in muscle strength. Thus, the mice were examined to determine whether ovarian cancer or WFA had an effect on forelimb grip strength. There was a steady reduction in normalized forelimb grip strength in the vehicle treated group compared to the control group, reaching statistical significance at week 2 post-xenografting of A2780 cells (F(3, 16)=11.01 (week 2); p=0.0003) (FIG. 2A). The mice treated with WFA showed a reduction in normalized grip strength compared to the control group, reaching statistical significance at week 3 post-implantation (F(3, 16)=26.64 (week 3); WFA 2 mg/kg: p=0.0003; WFA 6 mg/kg: p=0.0005). Despite the reduction, there was an attenuation of normalized forelimb grip strength following treatment of WFA compared to the vehicle treated group, achieving statistical significance by week 2 post-injection of A2780 cells (WFA 2 mg/kg: p=0.0058; WFA 6 mg/kg: p=0.0029). No statistical difference between both doses of WFA was found (p=0.9861). This experiment has subsequently been further refined in Example 2.

In a separate experiment the normalized 4-paw (total limb) grip strength was measured. The vehicle treated group showed a profound reduction in normalized total limb grip strength compared to the control group, achieving statistical significance by week 2 post-injection of A2780 cells (F(3, 16)=11.61 (week 2); p=0.0002) (FIG. 2B). Treatment with 2 mg/kg of WFA showed a significant protective effect on total limb grip strength throughout the entirety of the study compared to the vehicle treated group (F(3, 16)=3.480 (week 1), 11.61 (week 2), and 26.62 (week 3); p=0.03, 0.0039, and 0.005, respectively). Treatment with 6 mg/kg of WFA was initially protective, but the grip strength eventually deteriorated to the same extent as the vehicle treated group. Based on the increased mortality rate of the WFA 6 mg/kg and the decline in grip strength similar to that of the vehicle treated group, 6 mg/kg of WFA may be a lethal dose for these animals. This experiment has subsequently been further refined in Example 2.

In conjunction with a loss of muscle strength, a reduction in skeletal muscle mass is characteristic of cachexia. Changes were measured in the normalized weight of select muscles of the lower extremity, as shown in FIG. 2C. There was a significant reduction in normalized wet weight of the tibialis anterior (TA) of all tumor-bearing mice treated with WFA compared to the control group (F(3, 14)=8.876; WFA 2 mg/kg: p=0.01; WFA 6 mg/kg: p=0.001). The gastrocnemius (GA) of the WFA 2 mg/kg group showed a significant reduction in normalized wet weight compared to the control group (F(3, 14)=4.591; p=0.02). The quadriceps femoris (QF) of the WFA 6 mg/kg group displayed a significant reduction in wet weight compared to the control group (F(3, 14)=3.968; p=0.02). Surprisingly, no difference between the vehicle and WFA treated groups was found. To investigate the discrepancy between a reduction in grip strength and no changes in muscle weight between the vehicle and WFA treated groups, on transverse sections of the TA, GA, and QF muscles were stained with hematolylin & eosin (H&E) (FIG. 2D, TA images not shown) and the myofiber cross-sectional area (CSA) was measured (FIG. 2E). In all three skeletal muscles, there was a significant reduction in average myofiber CSA in the vehicle treated group compared to the control group (F(3, 14)=75.62 (TA), 37.58 (GA) and 51.29 (QF); p<0.0001 for all muscles). Both treatments of WFA resulted in CSAs that were significantly greater than the vehicle treated group for all three muscles examined (WFA 2 mg/kg: p<0.0001 (TA & GA), p=0.0003 (QF); WFA 6 mg/kg: p<0.0001 (TA), p=0.0002 (GA), p=0.004 (QF). In the TA and QF muscles, there was a significant reduction in average myofiber CSA in the WFA treated groups compared to the control group (WFA 2 mg/kg: p<0.0001 (TA & QF); WFA 6 mg/kg: p=0.0003 (TA), p<0.0001 (QF)). We found a restoration of myofibrillar CSA in the GA muscle of mice treated with 2 mg/kg of WFA (p=0.09), but not those treated with 6 mg/kg of WFA (p=0.007) compared to the control group. While a reduction in average CSA was found, there was only a weak trend towards an increase in the number of myofibers present in the tumor bearing groups that did not approach statistical significance. A scant degree of skeletal muscle edema was present in select muscles of tumor-bearing mice, but not to an extent that would mask weight changes. Thus, treatment with WFA partially rescues grip strength and myofibrillar CSA in this cachectic model, demonstrating a functional improvement in the xenografted cancer model. These experiments have subsequently been further refined in Example 2.

WFA Abolishes the Slow-to-Fast Myofiber-Type Conversion Associated with Cancer Cachexia.

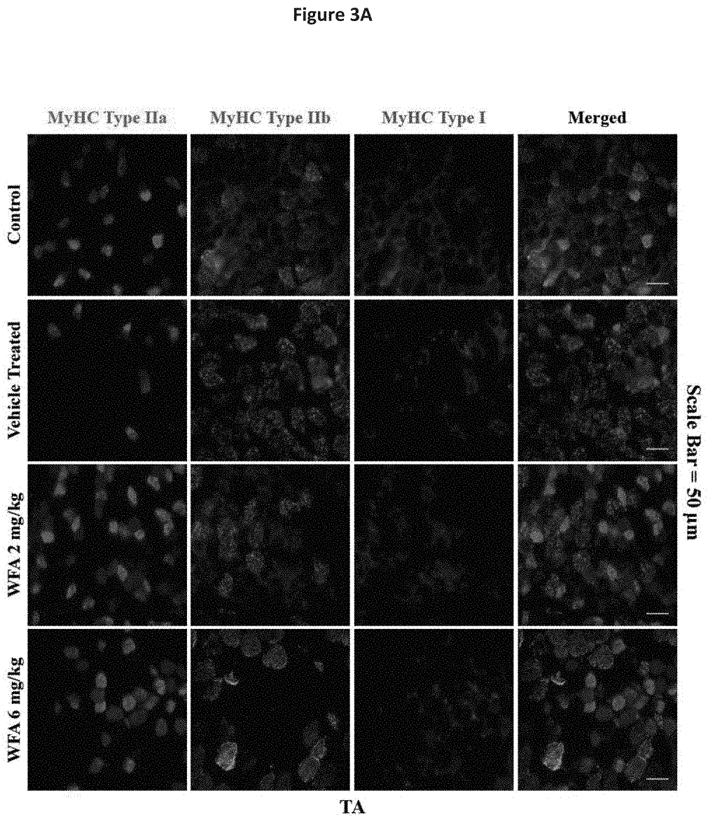

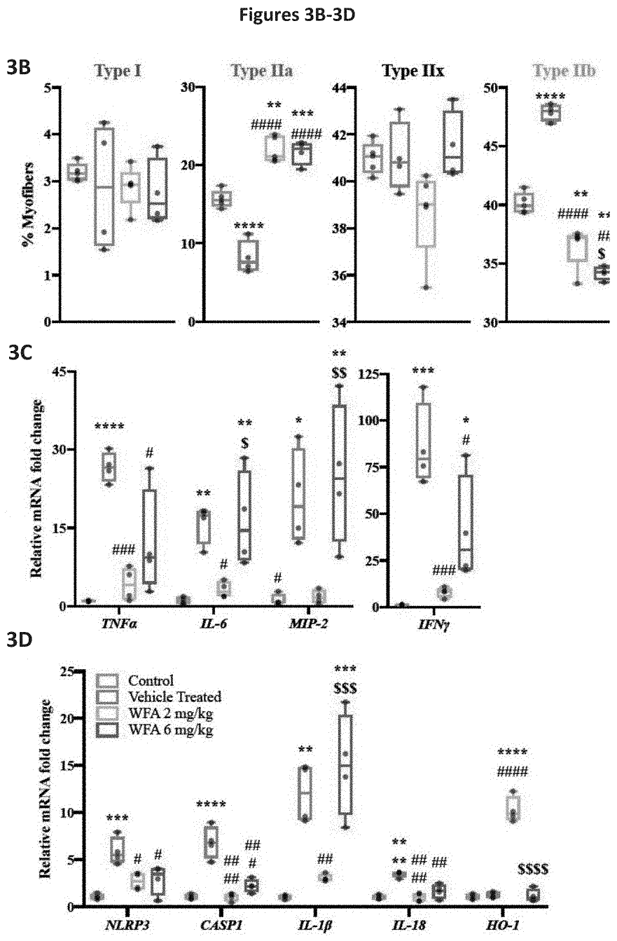

Skeletal muscle wasting in response to various pathological states, including cancer-induced cachexia, is associated with the conversion of slow skeletal muscle fibers (Type IIa) to fast skeletal muscle fibers (Type IIb) in certain muscles, such as the soleus and TA. Further, studies have shown that fast skeletal muscle fibers exhibit an accelerated rate of atrophy. To assess myofiber composition changes, immunohistochemistry (IHC) was used to detect expression of select isoforms of myosin heavy chain, as shown in FIG. 3A. Unstained myofibers were considered to be type IIx, which are generally considered to by myofibers that are transitioning fiber-type. Type I and IIa fibers were found clustered towards the inner aspect of the TA muscle and the myofiber distribution became predominantly Type IIb and Type IIx moving laterally from the centralized clusters. Type I myofibers made up a small amount of the myofibers in the TA muscle in all groups, and no significant differences were found (F(3, 14)=0.2942 with an associated p-value of 0.83). There was a significant reduction in the percentage of Type IIa myofibers in the vehicle treated group compared to all other groups (F(3, 14)=67.78; p<0.0001 for all comparisons), as shown in FIG. 3B. Interestingly, there was a significant increase in Type IIa myofibers in both groups treated with WFA compared to the vehicle treated and control groups (WFA 2 mg/kg: p=0.0001; WFA 6 mg/kg: p=0.0004). For the Type IIx myofibers, one-way ANOVA indicated a significant difference (F(3, 14)=3.603 with an associated p-value of 0.04). However, no significant differences were elucidated upon post hoc analysis. Opposite of the Type IIa myofibers, there was a significant increase in the proportion of Type IIb myofibers in the vehicle treated group compared to all groups (F(3, 14)=110.5; p<0.0001 for all comparisons). Further, the amount of Type IIb myofibers was significantly reduced in the WFA treated groups in a dose-dependent manner, as compared to the control (WFA 2 mg/kg: p=0.001; WFA 6 mg/kg: p<0.0001) and vehicle treated groups (p<0.0001 for both groups). Taken together, this Example demonstrates that ovarian cancer induces changes in skeletal muscle myofiber composition, which is ameliorated by treatment with WFA. Interestingly, treatment with WFA resulted in more Type IIa and fewer Type IIb myofibers than what was exhibited in the control group.

WFA Impedes Transcriptional Changes Associated with Myofiber-Type Conversion.

Studies have shown that systemic expression of tumor secreted proinflammatory cytokines downstream of the NF-κB pathway, and the NLRP3 inflammasome are capable of orchestrating myofiber atrophy and fiber-type conversion. Therefore, a study of classical proinflammatory cytokines downstream of the NF-κB pathway and components of the NLRP3 inflammasome was conducted. Relative mRNA levels of all proinflammatory cytokines assessed (TNFα, IFNγ, IL-6, and MIP-2) were significantly increased in the TA muscle of vehicle treated mice compared to the control group (F(3, 9)=24.09, 47.30, 178.2, and 555.1, respectively; p=0.0047 (TNFα), p<0.0001 for all other genes), as shown in FIG. 3C. Transcript levels of these inflammatory cytokines were significantly reduced in the WFA 2 mg/kg group compared to the vehicle treated group (p=0.02 (TNFα), p<0.0001 for all other genes), and were not significantly different than the control group (p=0.81, 0.81, 0.06, and 0.66, respectively). Curiously, we saw a robust increase in the expression of select proinflammatory cytokines in the WFA 6 mg/kg group compared to the control (p=0.001, 0.002, 0.002, and <0.0001, respectively) and WFA 2 mg/kg groups (p=0.0006, 0.0081, <0.0001, and <0.0001, respectively), likely as a byproduct of the toxicity of the high dosage. Relative transcript levels of IFNγ and MIP-2, the functional ortholog of human IL-8, were significantly reduced in the WFA 6 mg/kg group compared to the vehicle treated group (p=0.0034 and 0.0013, respectively), whereas relative transcript levels of TNFα and IL-6 were increased (p=0.09 and <0.0001, respectively). Along a similar line, the NLRP3 inflammasome components NLRP3, CASP1, IL-1β, and IL-18 were significantly increased in the vehicle treated group compared to the control group (F(3, 9)=27.51, 55.82, 89.30, and 10.87, respectively; p<0.0001, <0.0001, =0.0008, 0.0021, respectively), shown in FIG. 3D. Transcript levels of HO-1, an inhibitor of the NLRP3 inflammasome, were not significantly changed in the vehicle treated group compared to the control group (F(3, 9)=25.47; p=0.71). In the WFA 2 mg/kg group, we observed a significant reduction in all tested NLRP3 inflammasome transcript levels (p=0.0035, <0.0001, 0.02, and 0.002, respectively) with the exception of HO-1, which was increased compared to the vehicle treated group (p=0.0006). For the WFA 6 mg/kg group, we report no significant changes in HO-1 expression compared to the control or vehicle treated groups (p=0.99 and 0.56, respectively), and a significant decrease compared to the WFA 2 mg/kg group (p<0.0001). However, we found that the relative mRNA levels of NLRP3 and IL-18 were significantly reduced in the WFA 6 mg/kg group compared to the vehicle treated group (p=0.0008 and 0.01, respectively). Curiously, transcript levels of CASP1 and IL-1β were significantly increased in the WFA 6 mg/kg group compared to the control group (p=0.0003 and <0.0001, respectively). Collectively, our results indicate that low doses of WFA systemically downregulate classical downstream targets of the NF-κB pathway and the NLRP3 inflammasome at a transcriptional level, which are known to mediate myofibrillar atrophy and myofiber-type conversion.

WFA Inhibits the Production of NF-κB-Related Proinflammatory Cytokines in a Xenografted Model of Ovarian Cancer.

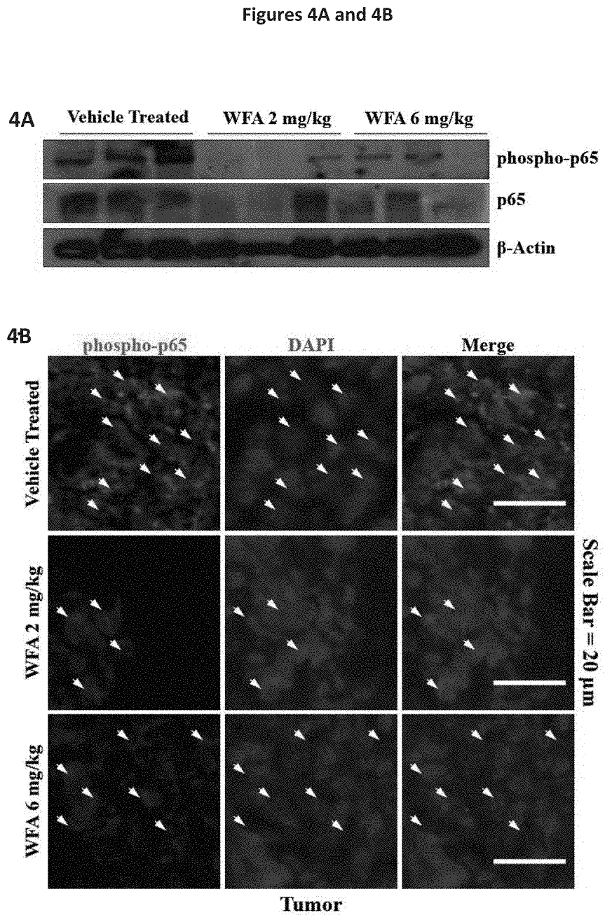

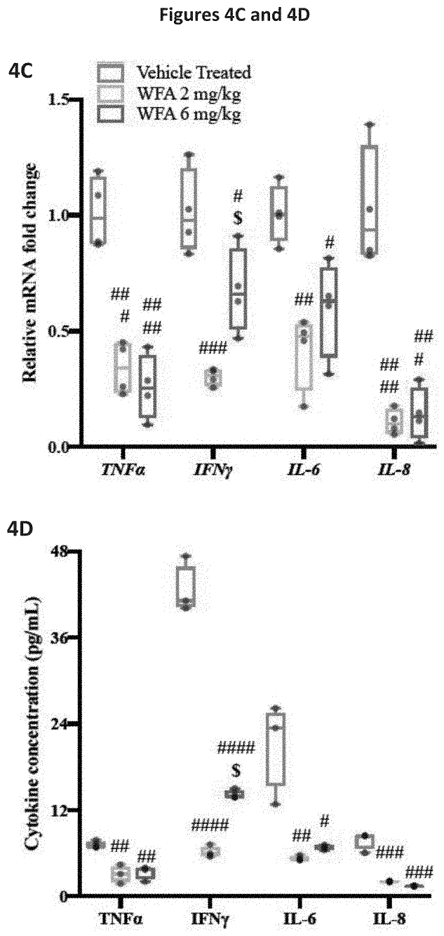

In the settings of ovarian cancer, proinflammatory cytokines linked to the NF-κB pathway are often upregulated. WFA has been reported to regulate the NF-κB pathway through inhibiting IKKβ activation and NF-κB-DNA binding. Therefore, we investigated whether various proinflammatory cytokines are being regulated in response to WFA treatment in the xenografted tumor. Tumor samples from the vehicle treated group showed the highest levels of phospho-p65, the active form of the canonical NF-κB signaling protein, at a protein level, shown in FIG. 4A. Treatment with WFA led to a robust decrease in the protein levels of phospho- and total p65. Tumor samples were assessed for the expression of phospho-p65 using IHC, shown in FIG. 4B. Phospho-p65 was found in nearly all of the tumor cells in the vehicle treated group and was severely diminished in both WFA treated groups. In addition to a reduction in phospho-p65 staining, there was a reduced amount of phospho-p65 co-localized with DAPI, indicating impairment in the translocation of p65 to the nucleus.

Expression of select NF-κB related proinflammatory cytokines is shown in FIG. 4C. Similar to the results in FIG. 3C, relative transcript levels of all proinflammatory cytokines assessed (TNFα, IFNγ, IL-6, and IL-8) were significantly reduced in the WFA 2 mg/kg treated group compared to the vehicle treated group (F(2,6)=8.518, 6.353, 5.893, and 7.434, respectively; p=0.02, 0.03, 0.04, and 0.02, respectively). Relative mRNA expression of TNFα was significantly reduced in the WFA 6 mg/kg treated group compared to the vehicle treated group (p=0.04). Relative expression of IL-6 and IL-8 genes showed a trend towards reduction (p=0.07 and p=0.09, respectively) in the WFA 6 mg/kg treated group, although it did not reach the level of statistical significance. As a final level of confirmation, a human MAP multiplex assay was performed to assess levels of select proinflammatory cytokines related to the NF-κB pathway (TNFα, IFNγ, IL-6, and IL-8; F(2, 6)=15.59, 201.4, 13.12, and 54.46, respectively). A low dose of WFA significantly reduced the expression of all proinflammatory cytokines tested compared to the vehicle treated group (p=0.006, <0.0001, 0.008, and 0.0004, respectively), as shown in FIG. 4D. The high dose of WFA resulted in a mixed result of up- and downregulation of proinflammatory cytokines compared to the vehicle treated group (p=0.008, <0.0001, 0.01, and 0.0002, respectively). Collectively, these results demonstrate that WFA modulates activation of the canonical NF-κB pathway and downstream transcriptional activities, indicating the mechanism through which WFA ameliorates cachectic signaling in our xenografted ovarian cancer model.

WFA Inhibits NLRP3 Inflammasome-Mediated Signaling in Xenografted Ovarian Tumors.

The downstream NF-κB signaling molecules 11-1β and TNFα are known to mediate the NLRP3 inflammasome, which is aberrantly expressed in certain oncological settings, such as ovarian cancer.

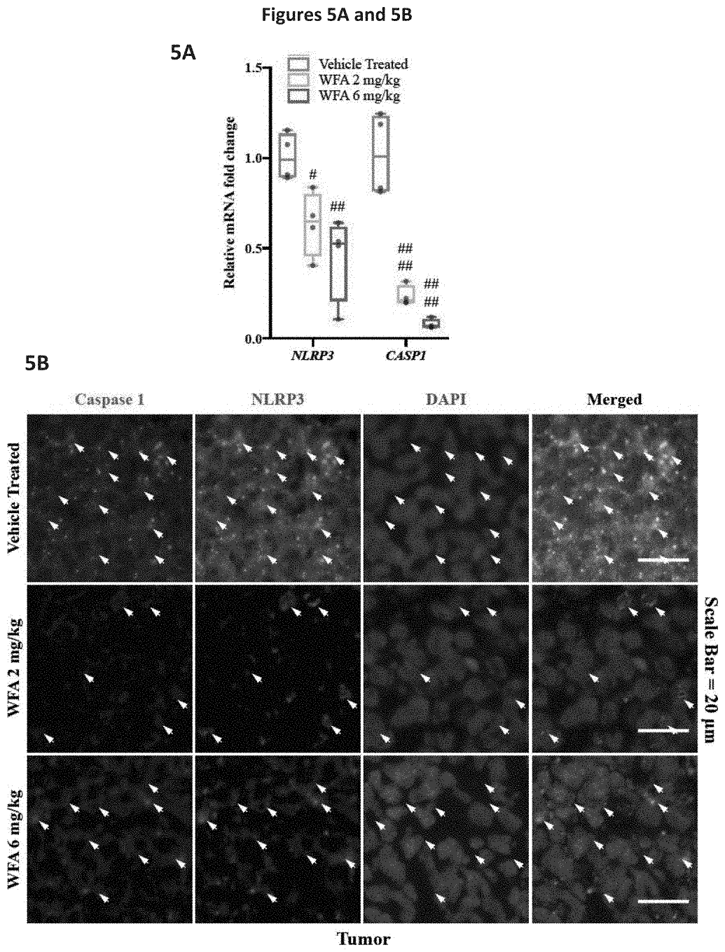

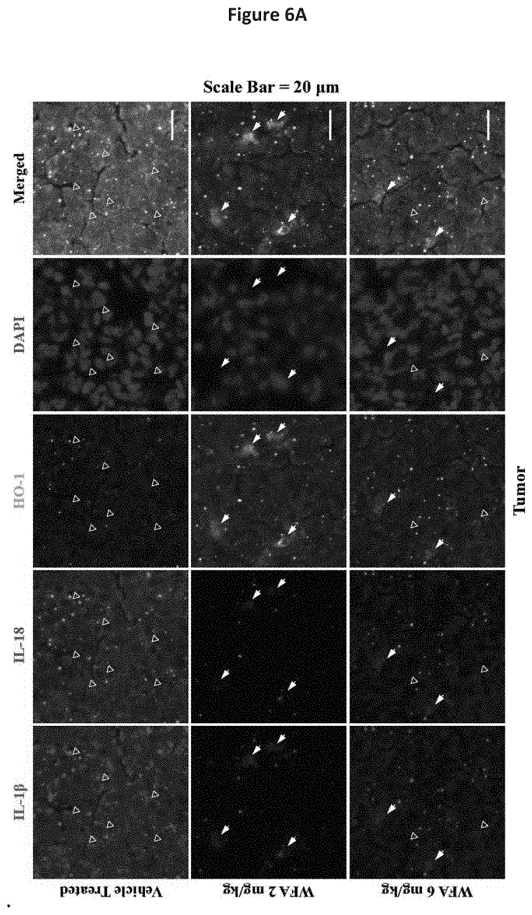

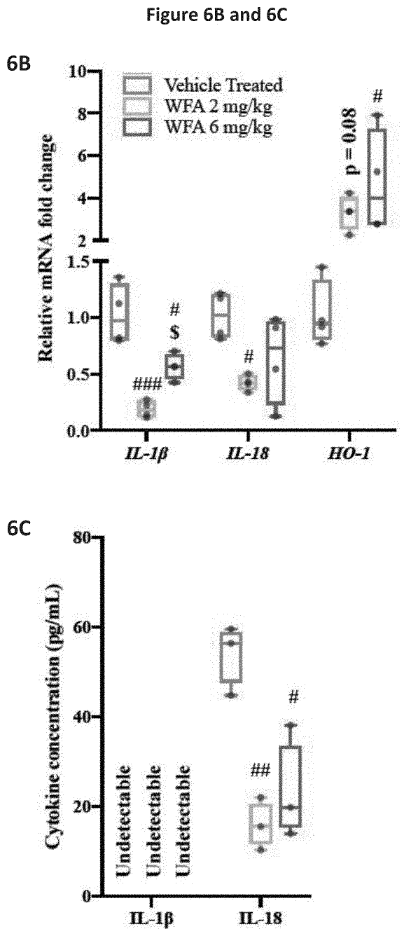

Therefore, the expression of select NLRP3 inflammasome markers (NLRP3, CASP1, IL-1β, IL-18, and HO-1; F(2, 6)=2.54, 181.7, 49.83, 3.90, and 8.27, respectively) was assessed in the xenografted tumors using real-time quantitative RT-PCR (qPCR). Relative mRNA levels of NLRP3 showed a reduction in both WFA treated groups (p=0.10 and p=0.11, respectively) compared to the vehicle treated group, shown in FIG. 5A. However, due to large variations from animal to animal, the reduction in NLRP3 mRNA levels was not found to be significant compared to vehicle treated group (WFA 2 mg/kg: p=0.19; WFA 6 mg/kg: p=0.22). In contrast, relative transcript levels of CASP1 showed a dose-dependent decrease compared to the vehicle treated group (WFA 2 mg/kg: p=0.002; WFA 6 mg/kg: p<0.0001). Relative transcript levels of the critical NLRP3 inflammasome component IL-1β were significantly reduced in both WFA treated groups compared to the vehicle treated group (WFA 2 mg/kg: p=0.001; WFA 6 mg/kg: p=0.003) (FIG. 6B). Relative gene expression of IL-18 showed a trend towards reduction (p=0.07) in the WFA 2 mg/kg group and did not approach the level of statistical significance in the WFA 6 mg/kg group (p=0.21), compared to the vehicle treated group, which is shown in FIG. 6B. Relative transcript levels of the inflammasome inhibitor HO-1 trended towards an increase in the WFA 2 mg/kg group (p=0.06) and were significantly increased in the WFA 6 mg/kg group (p=0.03) compared to the vehicle treated group, suggesting that WFA reduces ovarian cancer-induced cachexia through the regulation of NLRP3-dependent inflammasome.

To confirm the qPCR results, MC staining of tumor sections was used to assess change in protein levels of the NLRP3 inflammasome, which are shown in FIGS. 5B and 6A. FIG. 5B shows expression of Caspase 1 and NLRP3. There was a reduction in Caspase 1 and NLRP3 staining in both WFA treated groups compared to the vehicle treated group. Positive staining for Caspase 1 and NLRP3 was higher in the WFA 6 mg/kg compared to the WFA 2 mg/kg group. Following, the tumors were then assessed for detection of the key inflammasome executioners IL-1β and IL-18, as well as the NLRP3 inflammasome inhibitor HO-1, shown in FIG. 6A. The vehicle treated group displayed the highest expression of IL-1β and IL-18, and the lowest expression of HO-1. Treatment with 2 mg/kg drastically lowered the expression of IL-1β and IL-18 and increased the expression of HO-1. Surprisingly, when cells were found to be positive for all three proteins, the intensity of HO-1 was severely increased in the WFA treated groups. The WFA 6 mg/kg group showed an intermediate expression of all three proteins, consistent with our prior experiments indicating a phenotype that is selectively proinflammatory. In conjunction with the data collected in FIG. 4D, the levels of select inflammasome components in our human MAP multiplex were measured, but these were limited to detection of IL-1β and IL-18, shown in FIG. 6C. Protein levels of IL-1β fell below the threshold for detection. However, there was a significant reduction in the cytokine levels of IL-18 in both WFA treated groups compared to the vehicle treated group (F(2, 6)=13.90; WFA 2 mg/kg: p=0.006; WFA 6 mg/kg: p=0.02). The effect of WFA on NLRP3 inflammasome signaling in skeletal muscle was more compelling. However, there were modest changes in this pathway in the xenografted tumors following treatment with WFA.

Discussion

Cancer cachexia has been recognized as a severe byproduct of numerous cancers for several years, and negatively affects the survival outcome and quality of life of patients. A recent report analyzed the prevalence of cachexia in 14 different cancer types known to exhibit cachexia or a muscle wasting syndrome within the United States and the European Union, and found that over 1.3 million cancer patients exhibited clinical signs of cachexia in these oncological settings. Reports have shown that cachexia is mediated, at least in part, by canonical NF-κB signaling and the NLRP3 inflammasome.

Ovarian cancer is an ideal model to understand and target cachexia due to its upregulation of NF-κB signaling and high incidence of cachexia. A recent report established a xenograft model of ovarian cancer in mice, where the researchers' specific goal was to mimic the clinical symptoms of cachexia in ovarian cancer patients. See Pin et al. Growth of ovarian cancer xenografts causes loss of muscle and bone mass: a new model for the study of cancer cachexia. Journal of cachexia, sarcopenia and muscle 2018, 9(4):685-700. Indeed, Pin et al. report that xenografting of the ovarian cancer cell line ES-2 resulted in a profound cachectic phenotype, exhibiting gross body changes and functional muscle weakening, consistent with cachexia symptoms in humans. This Example validated an ovarian cancer xenograft model and then reversed the cancer-induced cachectic phenotype. This recapitulates the essence of the aforementioned report, but also provides evidence of amelioration of the cachectic state. Consistent with clinical symptoms of ovarian cancer and cachexia, the vehicle-treated mice showed gross body changes, a reduction in survival, and functional muscle decline due to tumor burden. Treatment with WFA significantly rescued many of these parameters).

WFA is known to inhibit the activation of the canonical NF-κB pathway at defined steps, specifically activation of the IKK complex and translocation of p65 to the nucleus. This Example elucidates signaling paradigms immediately distal of the IKK complex (FIGS. 3-6). A key consequence of sustained activation of the canonical NF-κB pathway is the ubiquitous production and release of proinflammatory cytokines, such as TNFα and IL-6. During the acute phase of muscle injury, activation of the NF-κB pathway and subsequent production of proinflammatory cytokines is initially beneficial to the injured microenvironment and facilitates the initial phases of the repair process. However, prolonged exposure to these proinflammatory cytokines/chemokines rapidly becomes detrimental to skeletal muscle. Cancers are notorious for their ubiquitous expression of proinflammatory cytokines, ovarian cancer being no exception.

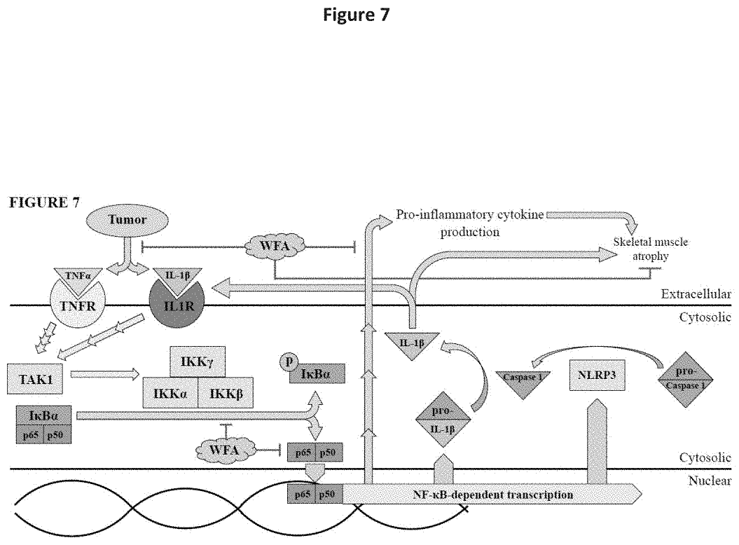

This Example demonstrates the molecular changes in the canonical NF-κB signaling pathway and the downstream consequences of this modulation. Without being bound by theory, FIG. 7 illustrates the mechanistic action of WFA and how it inhibits ovarian cancer-induced cachexia via a direct regulation of canonical NF-κB signaling and downstream effects blunting production of proinflammatory cytokines known to induce atrophying changes. The vehicle-treated group had a high expression of select proinflammatory cytokines (FIGS. 3 and 4). Treatment with WFA led to a robust decrease in the levels of these proinflammatory cytokines and reduced their transcriptional activation in the xenografted tumors, as well as in resident skeletal muscle. As a byproduct of this reduced inflammatory environment, the skeletal muscle myofibers were not atrophied to the extent as the vehicle treated group, displaying a promising treatment effect of WFA.

Another consequence of activating the canonical NF-κB pathway is the production of pro-IL-1β and pro-IL-18, which execute inflammasome-mediated cell death upon cleavage. Modulating the activity of the NF-κB pathway has also been shown to activate the NLRP3 inflammasome. In this signaling paradigm, NLRP3 leads to the proteolytic processing of pro-Caspase 1 to its mature form. Cleaved Caspase 1 in turn leads to the maturation of the previously produced pro-IL-1β and pro-IL-18. Recent reports have demonstrated a direct effect linking the NLRP3 inflammasome and skeletal muscle atrophy. See Huang et al., Deletion of Nlrp3 protects from inflammation-induced skeletal muscle atrophy. Intensive care medicine experimental 2017, 5(1):3. Consistent with this report, this Examples demonstrates a drastic increase in NLRP3 inflammasome components in the vehicle-treated group, which is reduced upon treatment with a therapeutic dosage of WFA (FIGS. 3, 5, and 6). The demonstrated reduction in proinflammatory signaling and concomitant improvements in skeletal muscle is consistent with published reports exhibiting an amelioration of cachectic signaling through skeletal muscle-specific inactivation of the NLRP3 inflammasome.

Ovarian cancers are known to induce expression of TNFα and IL-1β. Upstream of the IKK complex, TNFα and IL-1β signaling converges on the molecule TAK1. In addition to activating canonical NF-κB signaling, TAK1 activates the p38, JNK, and ERK MAPK pathways, all of which can influence skeletal muscle atrophy. Further distal of the NF-κB pathway are the ubiquitin proteasome system and autophagy signaling pathways. Proteolysis is orchestrated through these signaling paradigms and upregulation is frequently evidenced in cachectic states.

The totality of evidence reported herein suggests that WFA is a novel mediator of upstream cachectic signaling in our xenografted ovarian cancer model. Like resident skeletal muscle, cardiac muscle has been shown to undergo atrophy in response to cancer cachexia. It has been suggested that the mechanisms by which cardiac cachexia occur are through a similar mechanism as skeletal muscle cachexia. A published report by Mohanty et al. demonstrated that WFA is cardio-protective. See Mohanty et al. Mechanisms of cardioprotective effect of Withania somnifera in experimentally induced myocardial infarction. Basic & clinical pharmacology & toxicology 2004, 94(4):184-190.

### Example 2

Example 2 corroborates findings from Example 1 demonstrating that WFA treatment attenuates the atrophying and weakening effects of a xenograft model of ovarian cancer, elucidates the mechanisms by which ovarian cancer induces a cachectic phenotype, and further demonstrates that WFA improves functional muscle strength or size in a tumor-free setting. The effect of WFA is assessed on select critical regulators of skeletal muscle distal to the NF-κB signaling pathway, namely satellite cells and signaling to the UPS and ALS through the UPR.

Materials and Methods

Cell Line: as described in Example 1.

Generation of Tumor in Mice: Six-week old female NOD.Cg-Prkdc^{scid} Il2rg^{tm1Wjl}/SzJ (NSG, Jackson Lab Strain #005557) immunodeficient mice were initially randomly assigned to a tumor-free or tumor-bearing group (30 mice/group). Tumor-bearing groups received an IP injection of 8.0×10⁵ low passage A2780 cells suspended in 100 μl sterile PBS. Control group received IP injection of 100 μl sterile PBS alone. After an initial refractory period of eight days, mice in both the tumor-free and tumor-bearing groups were stratified into a group that would receive vehicle injections (10% dimethyl sulfoxide, 90% glycerol trioctanoate) or one of two concentrations of WFA (2 mg/kg (therapeutic dosage) or 4 mg/kg (potentially supratherapeutic dosage) via IP injection (10 mice/group). Post-euthanization, several tissues were collected, weighed, snap frozen in liquid nitrogen and then stored at −80° C. for further analysis. The mice were housed in a 12-hour light/dark cycle and given water and food ad libitum. The Institutional Animal Care and Use Committee (IACUC, protocol #15405) and Institutional Biosafety Committee (IBC, protocol #18-208) of the University of Louisville approved all experimental protocols in mice in advance, and the medium was changed every 48 hours.

Grip Strength Measurements: as in Example 1.

Histology and Morphometric Analysis of Skeletal Muscle: Select muscles of the lower limb of the mice were isolated, flash frozen in liquid nitrogen, mounted in O.C.T. embedding medium, and then sectioned using a microtome cryostat. To assess tissue morphology, 10 μm thick transverse sections were cut from the mid-belly of the tibialis anterior (TA) and then subjected to Hematoxylin and Eosin (H&E) staining. Images of H&E-stained TA muscle sections were quantified using Fiji software (National Institute of Health software) to measure myofiber cross-sectional area (CSA). Skeletal myofiber CSA was calculated by analyzing ˜500-700 myofibers per muscle as previously described (32).

Total RNA Extraction and qPCR: as in Example 1. Specific primers used are shown in Table 5.

TABLE 5
Gene specific primer sequences.

| Gene | Species | Direction | Nucleotide Sequence | SEQ ID NO |
|---|---|---|---|---|
| Paac7 | Mus | Forward | 5′-CAGTGTGCCATCTACCCATGCTTA-3′ | SEQ ID NO: 41 |
| | musculus | Reverse | 5′-GGTGCTTGGTTCAAATTGAGCC-3′ | SEQ ID NO: 42 |
| Myod1 | Mus | Forward | 5′-TGGGATATGGAGCTTCTATCGC-3′ | SEQ ID NO: 43 |
| | musculus | Reverse | 5′-GGTGAGTCGAAACACGGATCAT-3′ | SEQ ID NO: 44 |
| Eif2ak3 | Mus | Forward | 5′-ACTCCTGTCTTGGTTGGGTCTGAT-3′ | SEQ ID NO: 45 |
| | musculus | Reverse | 5′-CGTGCTCCGCTTATTCCTTTCT-3′ | SEQ ID NO: 46 |
| Atf4 | Mus | Forward | 5′-CTCTTCACGAAATCCAGCAGCA-3′ | SEQ ID NO: 47 |
| | musculus | Reverse | 5′-CCATGAGGTTTCAAGTGCTTGG-3′ | SEQ ID NO: 48 |
| Ddit3 | Mus | Forward | 5′-TGAAAGCAGAACCTGGTCCA-3′ | SEQ ID NO: 49 |
| | musculus | Reverse | 5′-CACTGTTCATGCTTGGTGCA-3′ | SEQ ID NO: 50 |
| Ppp1r15a | Mus | Forward | 5′-CAGAACATCAAGCCACGGAAGA-3′ | SEQ ID NO: 51 |
| | musculus | Reverse | 5′-AAAGTTGTCTCAGGTCCTCCTTCC-3′ | SEQ ID NO: 52 |
| Hspa5 | Mus | Forward | 5′-CGTGGAGATCATAGCCAACGAT-3′ | SEQ ID NO: 53 |
| | musculus | Reverse | 5′-ATTCCAAGTGCGTCCGATGA-3′ | SEQ ID NO: 54 |
| Hsp90B1 | Mus | Forward | 5′-GGGAGGTCACCTTCAAGTCG-3′ | SEQ ID NO: 55 |
| | musculus | Reverse | 5′-CTCGAGGTGCAGATGTGGG-3′ | SEQ ID NO: 56 |
| Ern1 | Mus | Forward | 5′-AAAGGTTCCGCTCATACAAAGGG-3′ | SEQ ID NO: 57 |
| | musculus | Reverse | 5′-TGAATGAAGCCAGCAGGAAGTTG-3′ | SEQ ID NO: 58 |
| Tnfrsf10B | Mus | Forward | 5′-AAGCCTTGCAGAGAGGGTATTGAC-3′ | SEQ ID NO: 59 |
| | musculus | Reverse | 5′-GCAGTTAGAGCATGACTGGCAGAT-3′ | SEQ ID NO: 60 |
| Fbxo30 | Mus | Forward | 5′-TCGTGGAATGGTAATCTTGC-3′ | SEQ ID NO: 61 |
| | musculus | Reverse | 5′-CCTCCCGTTTCTCTATCACG-3′ | SEQ ID NO: 62 |
| Fbxo32 | Mus | Forward | 5′-AAGGCTGTTGGAGCTGATAGCA-3′ | SEQ ID NO: 63 |
| | musculus | Reverse | 5′-CACCCACATGTTAATGTTGCCC-3′ | SEQ ID NO: 64 |
| Trim63 | Mus | Forward | 5′-TAACTGCATCTCCATGCTGGTG-3′ | SEQ ID NO: 65 |
| | musculus | Reverse | 5′-TGGCGTAGAGGGTGTCAAACTT-3′ | SEQ ID NO: 66 |
| Traf6 | Mus | Forward | 5′-TCGCCTAGTAAGACAGGACCATCA-3′ | SEQ ID NO: 67 |
| | musculus | Reverse | 5′-TGGTTCCATTTCGGCAACCT-3′ | SEQ ID NO: 68 |
| Sqstm1 | Mus | Forward | 5′-AGCACAGGCACAGAAGACAAGAGT-3′ | SEQ ID NO: 69 |
| | musculus | Reverse | 5′-AATGTGTCCAGTCATCGTCTCCTC-3′ | SEQ ID NO: 70 |
| Map1lc3b | Mus | Forward | 5′-CTGGTGAATGGGCACAGCATG-3′ | SEQ ID NO: 71 |
| | musculus | Reverse | 5′-CGTCCGCTGGTAACATCCCTT-3′ | SEQ ID NO: 72 |
| Becn1 | Mus | Forward | 5′-TGAAATCAATGCTGCCTGGG-3′ | SEQ ID NO: 73 |
| | musculus | Reverse | 5′-CCAGAACAGTATAACGGCAACTCC-3′ | SEQ ID NO: 74 |
| β-Actin | Mus | Forward | 5′-CAGGCATTGCTGACAGGATG-3′ | SEQ ID NO: 39 |
| | musculus | Reverse | 5′-TGCTGATCCACATCTGCTGG-3′ | SEQ ID NO: 40 |
| XBP1 | Mus | Forward | 5′-TTACGGGAGAAAACTCAGGGC-3′ | SEQ ID NO: 75 |
| (u/s) RT | musculus | Reverse | 5′-GGGTCCAACTTGTCCAGAATGC-3′ | SEQ ID NO: 76 |
| XBP1 | Mus | Forward | 5′-TGGAGCAGCAAGTGGTGGATTT-3′ | SEQ ID NO: 77 |
| (total) | musculus | Reverse | 5′-TGTCCATTCCCAAGCGTGTTCT-3′ | SEQ ID NO: 78 |
| RT | | | | |

Protein Extraction and Western Blotting: Quadriceps femoris (QF) samples were homogenized in chilled RIPA buffer (Sigma) supplemented with a Complete Mini Protease Inhibitor tablet (Roche Molecular Biochemicals, Indianapolis, Ind.). Tissue lysates were centrifuged at 10,000 RPM and the supernatants were collected. Protein concentration for each sample was determined using the Bradford reagent method (Bio-Rad), according to manufacturer's instructions. Protein lysates (50 μg) were separated on 10% SDS-PAGE gels at 100 volts for 2 h. The proteins were transferred to nitrocellulose membranes at 100 volts for 90 minutes. The membranes were blocked with 5% non-fat milk in Tris Buffered Saline supplemented with Tween-20 (0.5%; TBS-T) for 30-60 minutes. The membranes were then washed 3 times with TBS-T for 5 minutes. The membranes were then incubated with primary antibody at 4° C. overnight. The membranes were washed 3 times with TBS-T for 5 minutes each followed by incubation in TBS-T containing horseradish peroxidase conjugated secondary antibody (1:2000 dilution) for 1 h. The membranes were rinsed 3 times with TBS-T for 5 minutes each. Visualization of immunoreactive bands was enhanced using chemiluminescence reagents (Sigma). The membranes were stripped off using Restore™ Western Blot Stripping Buffer (Thermo Scientific Catalog #21059) for 30 minutes and re-probed with horseradish peroxidase-conjugated β-Actin used as a control to normalize the loading variation. Refer to Table 6 for a complete list of antibodies utilized.

TABLE 6
Primary and secondary antibodies.

| Antibody | Company | Catalog # | Application | Dilution |
|---|---|---|---|---|
| Pax7 (IgG1) | Santa Cruz | sc-81648 | IHC | 1:10 |
| MyoD (IgG2b) | Santa Cruz | sc-377460 | IHC | 1:20 |
| Laminin (Rabbit) | Sigma Aldrich | L9393 | IHC | 1:100 |
| Anti-mouse IgG1 CF594 | Sigma Aldrich | SAB4600326 | IHC | 1:2000 |
| Goat anti-rabbit Alexa | Thermo Fisher | A-11008 | IHC | 1:2000 |
| Fluor 488 | Scientific | | | |
| Goat anti-mouse IgG2b | Thermo Fischer | A-21143 | IHC | 1:2000 |
| Alexa Fluor 546 | Scientific | | | |
| DAPI | Roche | 10236276001 | IHC | 1:5000 |
| Phospho-PERK (Thr980) | Invitrogen | MA5-15033 | WB | 1:500 |
| PERK | Santa Cruz | sc-377400 | WB | 1:1000 |
| CHOP | ProteinTech | 60304 | WB | 1:1000 |
| Phospho-IRE1α (p- | GeneTex | GTX132808 | WB | 1:500 |
| Ser724) | | | | |
| IRE1α (HRP Linked) | Santa Cruz | sc-390960 HRP | WB | 1:1000 |
| Ubiquitin | Cell Signaling | 3933 | WB | 1:2000 |
| | Technologies | | | |
| LC3B | Cell Signaling | 3868 | WB | 1:1000 |
| | Technologies | | | |
| Beclin 1 (HRP Linked) | Santa Cruz | sc-48341 HRP | WB | 1:1000 |
| p62 (HRP Linked) | Santa Cruz | sc-48402 HRP | WB | 1:1000 |
| GAPDH (HRP Linked) | Cell Signaling | 8884 | WB | 1:1000 |
| | Technologies | | | |
| β-Actin (HRP Linked) | Sigma Aldrich | A3854 | WB | 1:2000 |
| ECL Donkey anti-rabbit | Millipore Sigma | GENA934 | WB | 1:3000 |
| HRP Linked Whole Ab | | | | |

IHC—immunohistochemistry;
WB—Western Blotting

Imaging. Slides were mounted using Eukitt Quick-hardening mounting medium (Sigma-Aldrich) and visualized at −0.4° C. on a Nikon TiE 3000 inverted microscope (Nikon) equipped with a digital camera (DS-U2/L2-Ri1 digital microscope camera (Nikon) for light microscopy or DXM-1200C coded digital camera (Nikon) for fluorescent microscopy), and Nikon NIS Elements AR software (Nikon). Exposure times were consistent for each staining type. Image levels were equally adjusted using Adobe Photoshop CS6 software (Adobe) to remove nonspecific background staining. Margins of cropped images are indicated by a dashed red border.

Graphical Display and Statistical Analysis: For the sake of transparency, the majority of the results were expressed as box-and-whisker plots with the box comprised of the first, second, and third quartiles, and the lower and upper whiskers corresponding to the minimum and maximum values, respectively, to display the entire range of data. Individual data points are depicted as black circles. Cropping of images is indicated by a solid black or white line around the field of view. Statistical analysis of the data was performed using an unpaired two-tailed t-test with Welch's correction for simple two group comparisons, a one-way analysis of variance (ANOVA) followed by Tukey's Honestly Significant Difference Test (HSDT) post hoc analysis for comparisons between 3 or more groups with one experimental factor, or a two-way ANOVA followed by Tukey's multiple comparison test post hoc analysis for comparisons between 4 or more groups containing two experimental factors to determine statistically significant differences between groups with GraphPad Prism 8.3.0 software for Mac (La Jolla, Calif., USA). ANOVA summaries presented in supplementary information. Welch-corrected or Tukey-corrected p-value of <0.05 was considered statistically significant, unless otherwise specified.

Results

Withaferin A Improves Basal Grip Strength and Attenuates the Effects of Ovarian Cancer on Skeletal Muscle.

Recent work from our lab has demonstrated that the A2780 ovarian cancer cell line is capable of inducing a skeletal muscle cachectic phenotype and that WFA attenuates these changes (28). In the present study, the effect of WFA on functional muscle strength and the size of muscle in tumor-free mice corroborated prior results in tumor-bearing mice using the same lower dosage of WFA (2 mg/kg), but a different upper dosage of WFA (4 mg/kg) due to the deleterious effects previously observed with 6 mg/kg of WFA. Additionally, due to the previously reported tumor-associated mortality, the amount of xenografted cells was reduced from 1×106 to 8×105. A discussion of gross body changes, tumor burden, and the effect on cardiac muscle following xenografting of the A2780 ovarian cancer cell line has recently been discussed in the work by Kelm et al. (2020)

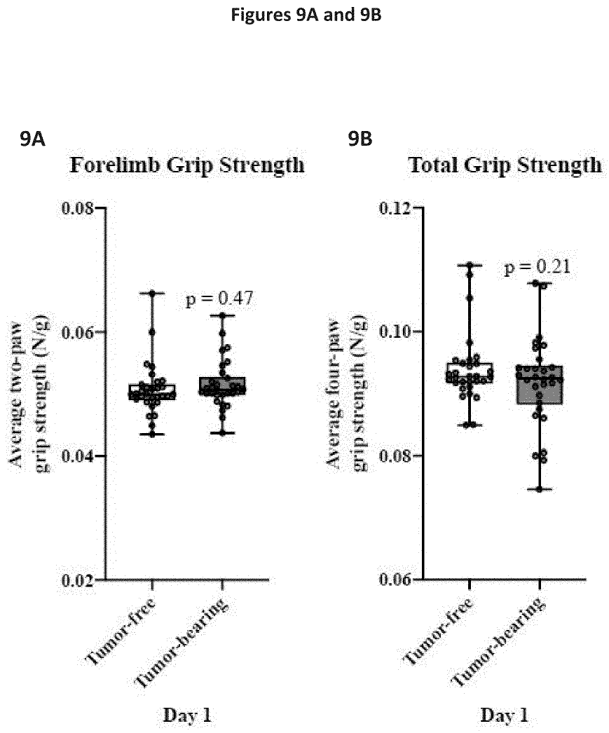

Before the mice received IP xenografts of the A2780 ovarian cancer cells, forelimb and total grip strength analyses were performed to ensure no significant differences within the groups stratified to become tumor-free or tumor-bearing existed at the start of the study. No significant differences were found in the baseline forelimb (tumor-free: 0.051±0.004 N/g; “tumor-bearing”: 0.052±0.004 N/g), shown in FIG. 9A, or total grip strength (tumor-free: 0.094±0.006 N/g; “tumor-bearing”: 0.092±0.007 N/g), shown in FIG. 9B, in the groups that were initially stratified to become tumor-free or tumor-bearing animals, as determined by an unpaired two-tailed Welch-corrected t-test (forelimb: t(57.63)=0.74, p=0.47; total limb: t(54.92)=1.27, p=0.21).

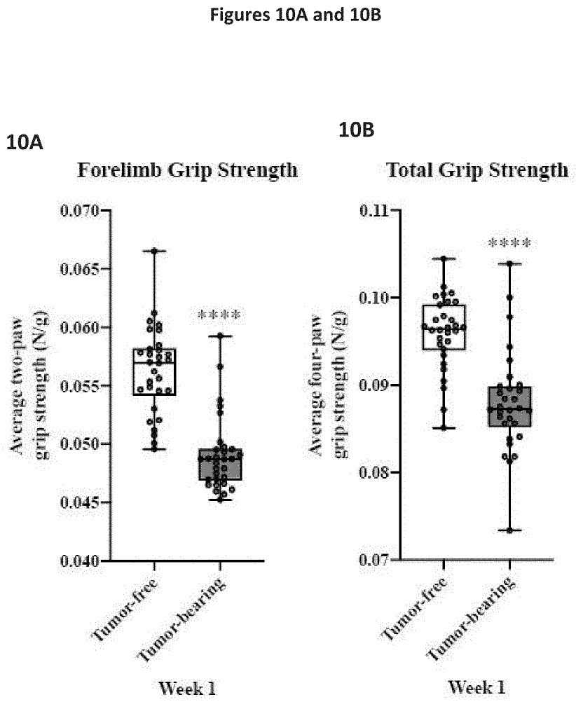

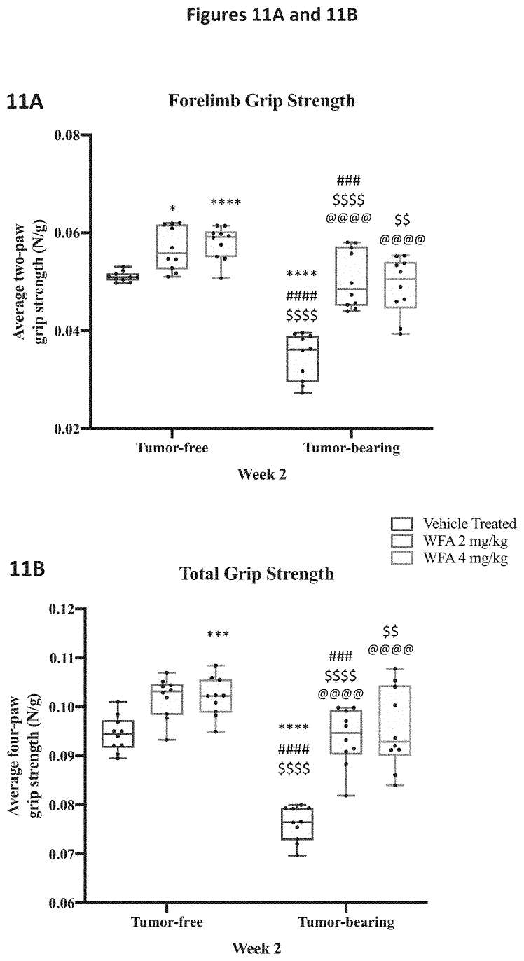

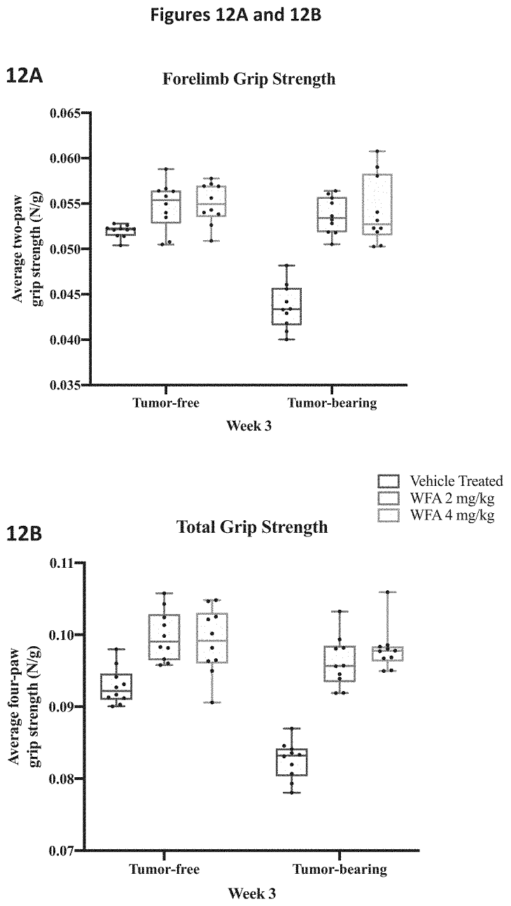

One-week post-xenografting, before WFA treatment was initiated, a significant reduction in the forelimb (tumor-free: 0.056±0.003 N/g; tumor-bearing: 0.049±0.003 N/g) and total grip strengths (tumor-free: 0.096±0.004 N/g; tumor-bearing: 0.088±0.006 N/g) of the tumor-bearing mice was observed compared to the tumor-free control group (forelimb: t(56.62)=8.02; total limb: t(52.64)=6.03; p<0.0001 for both comparisons), suggesting a rapid onset of muscle decline, as shown in FIGS. 10A and 10B, respectively. The same grip strength analyses of forelimb and total grip strengths were performed again at week 2 post-xenografting are presented in FIGS. 11A and 11B, respectively, and also at week 3 post-xenografting, as shown in FIGS. 12A and 12B, respectively. The intermediate timepoints demonstrated similar trends and p-values as the terminal week of the study.

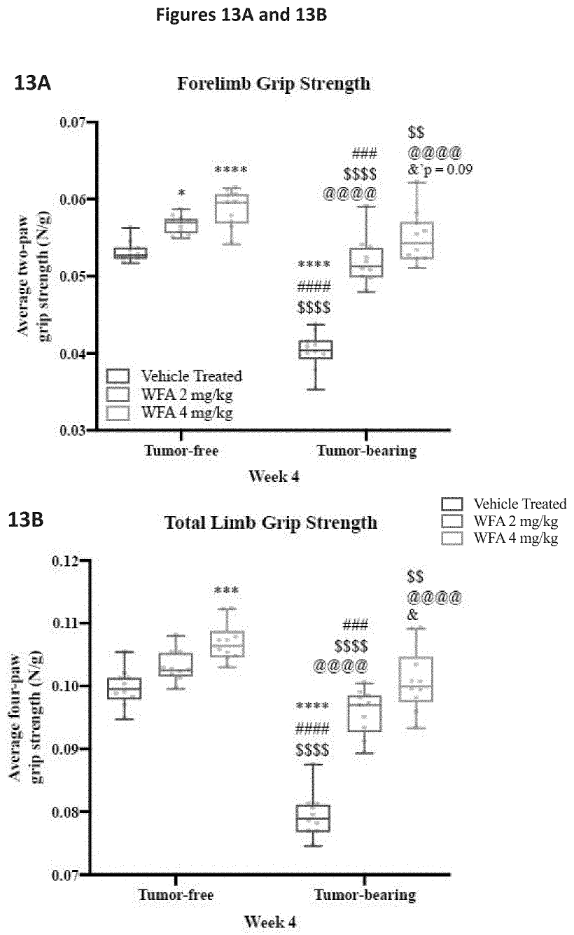

At the terminal week of the study, treatment with WFA at 2 mg/kg and 4 mg/kg significantly improved the forelimb grip strength of tumor-free mice (FIG. 13A), and the higher dosage of WFA had significantly improved the total grip strength of the tumor-free mice (FIG. 13B) compared to the tumor-free vehicle-treated group as determined by a two-way ANOVA followed by Tukey's multiple comparison test post hoc analysis (forelimb—WFA 2 mg/kg: p=0.02; WFA 4 mg/kg: p<0.0001; total limb—WFA 2 mg/kg: p=0.30; WFA 4 mg/kg: p=0.0005), suggesting that WFA treatment improves basal grip strength. The forelimb and total limb grip strength of the tumor-bearing vehicle-treated group was significantly reduced compared to the tumor-free groups (p<0.0001 for all comparisons). The tumor-bearing WFA-treated groups displayed forelimb and total grip strengths that were significantly increased compared to the tumor-bearing vehicle-treated (p<0.0001 for all comparisons), but not significantly different than the tumor-free vehicle-treated group, corroborating previous findings that WFA ameliorates the weakening effects on skeletal muscle in the xenograft model of ovarian cancer-induced cachexia. As stated above, grip strength analyses for week 2 and week 3 post-xenografting, presented in FIGS. 11 and 12, respectively, demonstrated similar trends and p-values as the terminal week of the study.

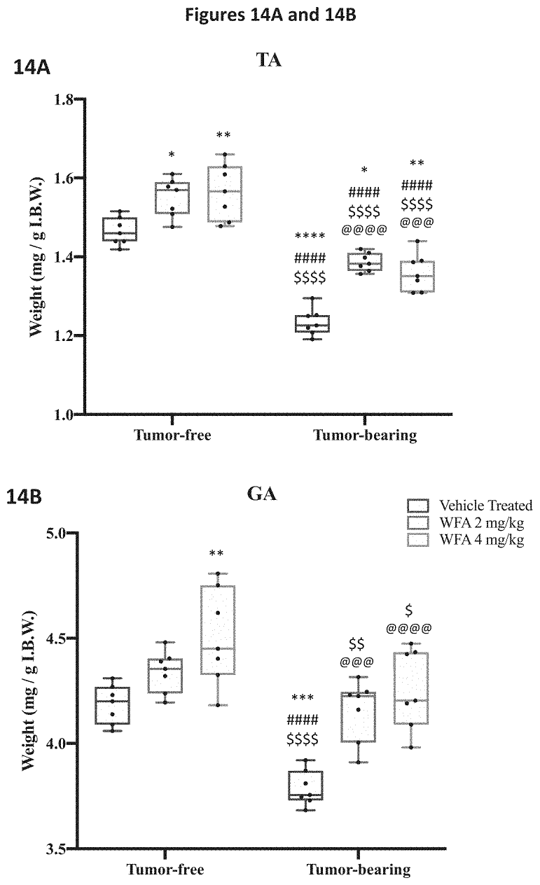

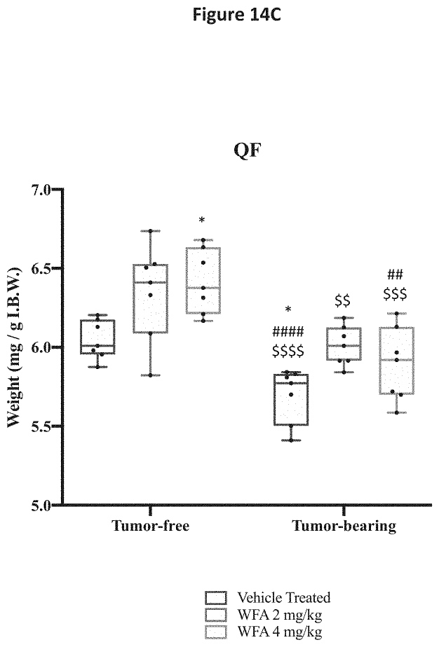

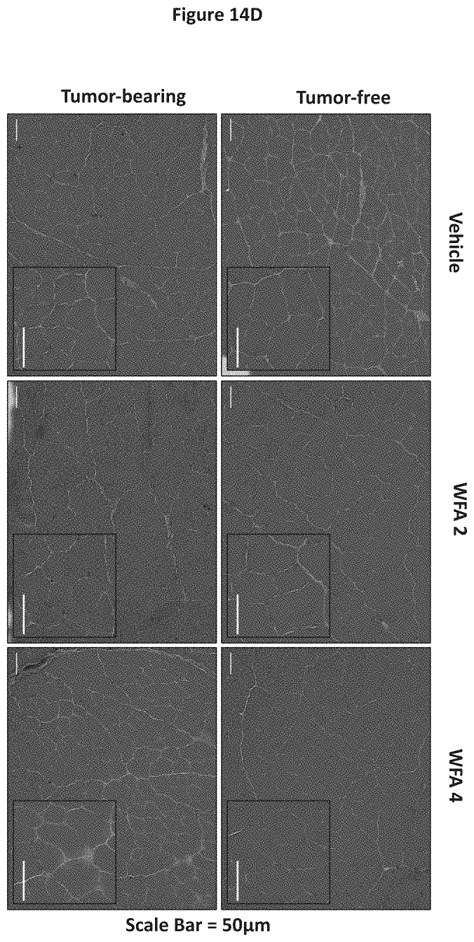

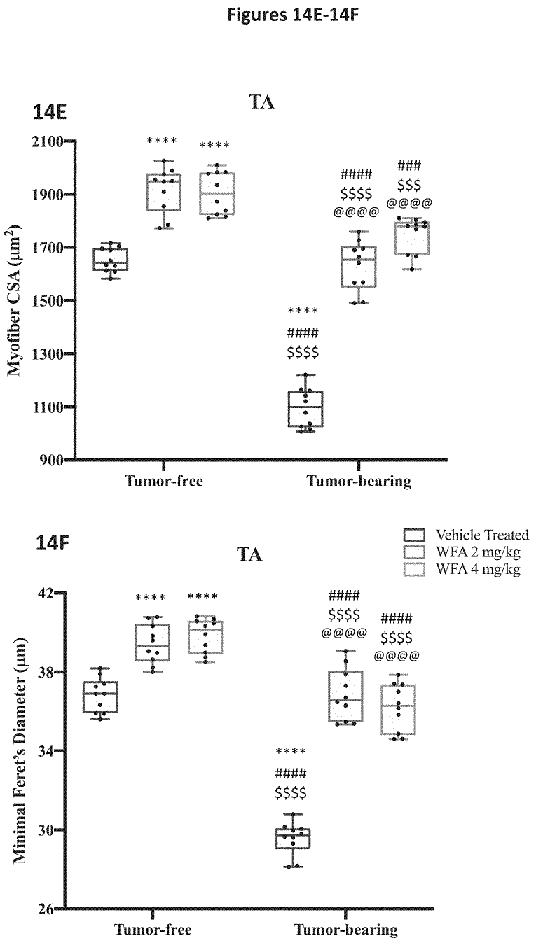

Post-mortem, select muscles of the lower extremities, including tibialis anterior (TA), gastrocnemius (GA), and quadriceps femoris (QF), were collected and weighed to detect changes in muscle mass, with results shown in FIG. 14A-14C, respectively. Muscle weights were normalized by the initial body weight (I.B.W.) to account for differences in the size of the mouse/muscle at baseline, while excluding the confounding effect of tumor burden on body weight. A reduction in the normalized weight of the TA, GA, and QF muscles was observed in the tumor-bearing vehicle-treated group compared to the tumor-free vehicle-treated group (p<0.05 for all comparisons). Within the tumor-free groups, the WFA 4 mg/kg group displayed a significant increase in normalized weight of the TA, GA, and QF muscles compared to the vehicle-treated group (p<0.05 for all comparisons). Within the tumor-bearing groups, treatment with both concentrations of WFA led to a statistically significant increase in normalized wet weight of the TA and GA muscles compared to the vehicle-treated group.

To corroborate the changes in muscle strength and weight in response to WFA treatment and the ovarian tumors, changes in myofibrillar size in the TA muscle were examined via Hematoxylin and Eosin staining, as shown in FIG. 14D. Similar to the changes in grip strength observed, both concentrations of WFA led to a significant increase in the average myofibrillar cross-sectional area (CSA) in the TA muscle of the tumor-free WFA-treated groups (WFA 2 mg/kg: 1916.39±85.72 μm2; WFA 4 mg/kg: 1904.98±80.50 μm²) compared to the tumor-free vehicle-treated group (1652.48±46.13 μm²) (p<0.0001 for both comparisons) (FIG. 14E). The average CSA of the tumor-bearing vehicle-treated group (1097.28±74.60 μm2) was significantly decreased compared to all tumor-free groups (p<0.0001 for all comparisons), shown in FIG. 14E. Within the tumor-bearing groups, both dosages of WFA led to a significant rescue in myofibrillar CSA (WFA 2 mg/kg: 1629.72±94.96 μm2; WFA 4 mg/kg: 1748.13±68.90 μm2) compared to the tumor-bearing vehicle-treated group (p<0.0001 for both comparisons). The average myofibrillar CSA of the tumor-bearing WFA-treated groups was not significantly different from that of the tumor-free vehicle-treated group (WFA 2 mg/kg: p=0.99; WFA 4 mg/kg: p=0.07). To confirm accurate measurement of the CSA, the minimal Feret's diameter was measured in conjunction with the CSA, with results shown in FIG. 14F. Nearly identical trends and levels of significance in the minimal Feret's diameter were observed compared to the CSA, indicating the validity of our histological assessment and corroborating the myofibrillar changes induced by WFA treatment and ovarian cancer.

Withaferin A Modulates Satellite Cell Activation.

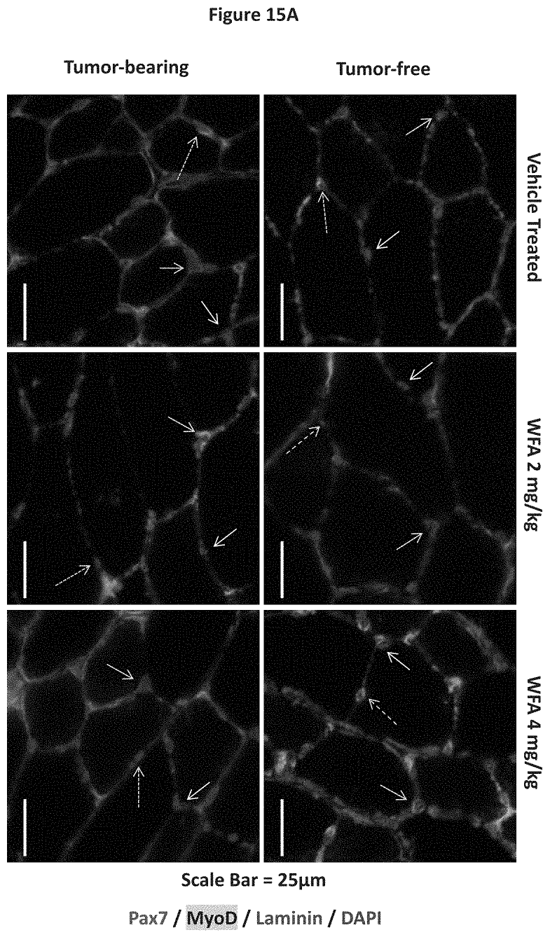

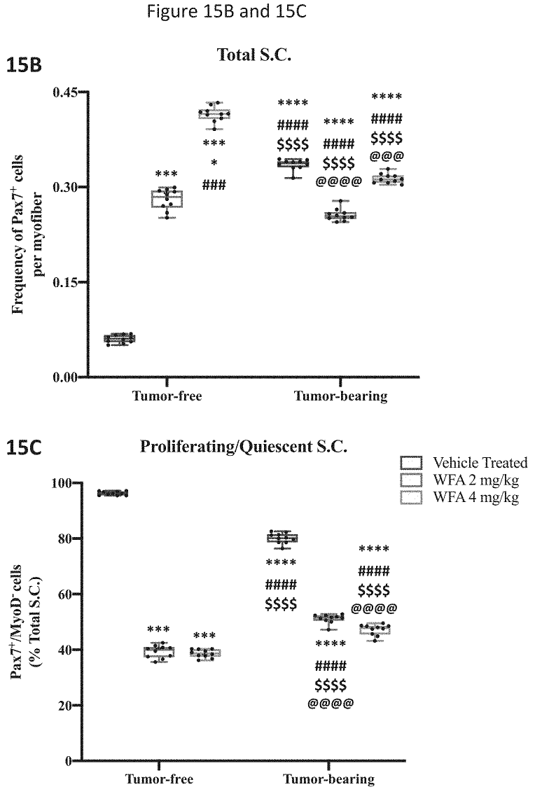

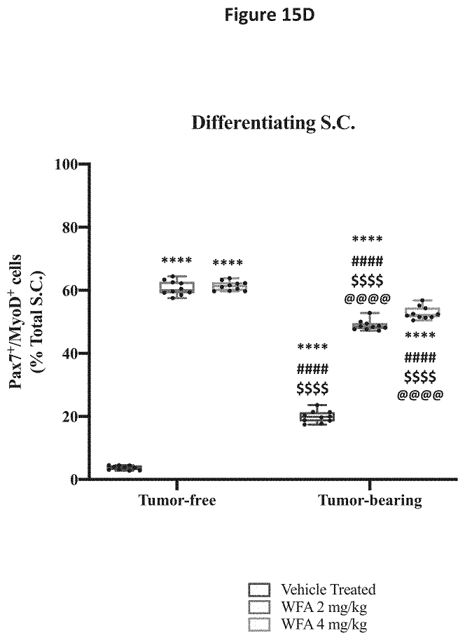

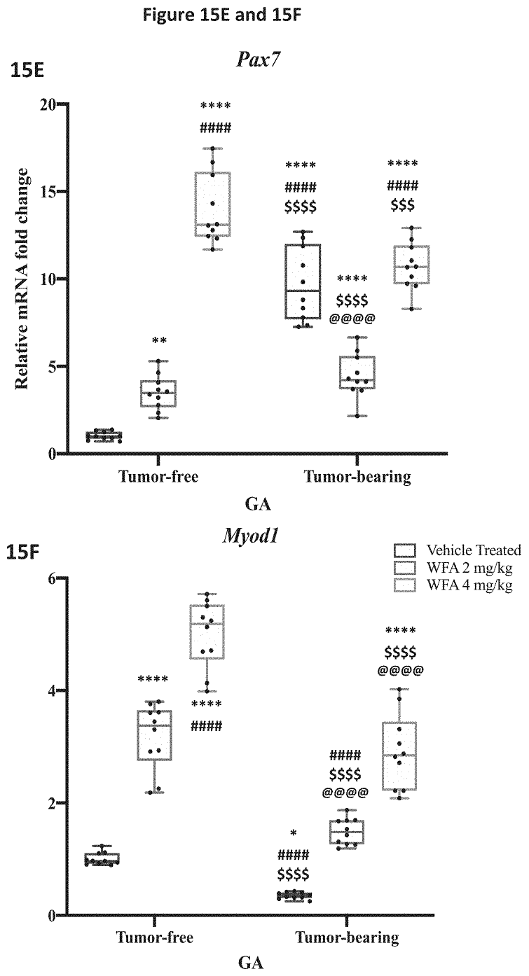

Dysregulation of NF-κ B signaling has been shown to spuriously activate satellite cells and lead to an impairment of skeletal muscle repair (8, 32). Due to the role of WFA in inhibiting canonical NF-κ B signaling and the effect ovarian cancer has on this signaling pathway, WFA and the xenograft model were tested to measure induction or activation of satellite cells. The TA muscle was subjected to immunofluorescent immunohistochemical (IHC) staining for Pax7 (a marker of satellite cells), MyoD (a marker of myogenic differentiation), and Laminin (a marker of the basal lamina) (FIG. 15A). As accurate IHC detection of satellite cells can be challenging, individual color channels were compared against negative control slides to optimize visualization of satellite cells, minimize nonspecific background staining, and rule out false positives (Pax7⁺/DAPI− cells) (37).

Initially, the number of satellite cells per field (normalized by the number of Laminin+ myofibers) was quantified to assess whether the model of ovarian cancer or WFA treatment affected the satellite cell population at large. Within the tumor-free groups, WFA treatment significantly increased the normalized amount of Pax7+ cells (WFA 2 mg/kg: 0.28±0.02; WFA 4 mg/kg: 0.42±0.01) compared to the tumor-free vehicle-treated group (0.06±0.01) (p<0.0001 for both comparisons), as shown in FIG. 15B. A significant increase in the amount of Pax7+ cells was observed in the tumor-free WFA 4 mg/kg group compared to the tumor-free WFA 2 mg/kg group (p<0.0001). There was a significant increase in the amount of Pax7+ cells in the tumor-bearing vehicle-treated group (0.34±0.01) compared to the tumor-free vehicle-treated and tumor-free WFA 2 mg/kg groups, although there was a significant reduction compared to the tumor-free WFA 4 mg/kg group (p<0.0001 for all comparisons). Interestingly, within the tumor-bearing groups, the WFA 2 mg/kg group displayed a significant reduction in Pax7+ cells (0.26±0.01) compared to the vehicle-treated and WFA 4 mg/kg groups (0.31±0.01) (p<0.0001 for both comparisons). A small, but statistically significant decrease in the tumor-bearing WFA 4 mg/kg group was observed compared to the tumor-bearing vehicle-treated group (p=0.0004). The tumor-free WFA 4 mg/kg group displayed the highest proportion of Pax7+ cells, possibly suggesting that WFA is a potent activator of satellite cells. Due to the systemic nature of WFA treatment, the tumor-specific uptake of WFA may have diminished the effect observed on skeletal muscle with respect to satellite cell activation in the tumor-bearing animals compared to the tumor-free animals.

After assessing changes in the gross number of satellite cells, the myogenic status was assessed to determine if the satellite cells were functionally activated. Satellite cells that are Pax7+/MyoD− are self-renewing or returning to quiescence, whereas satellite cells that are Pax7+/MyoD+ have committed to the myogenic lineage and will facilitate in muscle repair (6, 38). The vast majority of nuclei within and outside of myofibers were found to be Pax7−/MyoD+, and as such were not considered myogenic progenitors. It was not feasible to enumerate the population of Pax7−/MyoD+ cells in response to the xenografted cancer or WFA treatment due to experimental limitations. However, distinct populations of Pax7+/MyoD− and Pax7+/MyoD+ cells were present (FIGS. 15C and 15D). The majority of the satellite cells in the tumor-free vehicle-treated group were Pax7+/MyoD− (96.29±0.69%) and a small population was found to be Pax7+/MyoD+(3.71±0.69%). The proportion of proliferating satellite cells in the tumor-bearing vehicle-treated group (80.10±1.88%) was significantly decreased and the proportion of differentiating satellite cells (19.90±1.88%) was significantly increased compared to the tumor-free vehicle-treated group. However, the proportion of proliferating satellite cells was significantly increased and the proportion of differentiating satellite cells was significantly decreased compared to the tumor-free and tumor-bearing WFA-treated groups (p<0.0001 for all comparisons). In the tumor-free groups, there was a significant decrease in the proportion of proliferating satellite cells (WFA 2 mg/kg: 39.27±2.28%; WFA 4 mg/kg: 38.63±1.49%) and a significant increase in differentiating satellite cells (WFA 2 mg/kg: 60.73±2.28%; WFA 4 mg/kg: 61.37±1.49%) in the WFA-treated groups compared to the vehicle-treated group (p<0.0001 for all comparisons). In the tumor-bearing groups, there was a significant decrease in the proportion of proliferating satellite cells (WFA 2 mg/kg: 51.15±1.63%; WFA 4 mg/kg: 47.21±1.99%) and a significant increase in differentiating satellite cells (WFA 2 mg/kg: 48.85±1.63%; WFA 4 mg/kg: 52.79±1.99%) in the WFA-treated groups compared to the tumor-free and tumor-bearing vehicle-treated groups (p<0.0001 for all comparison).

To corroborate the IHC data, qPCR was performed on GA muscle extracts to assess relative transcript levels of Pax7 and Myod1 (FIGS. 15E and 15F). Similar to the IHC data, the tumor-bearing vehicle treated group displayed a significant increase in relative transcript levels of Pax7 and a significant decrease in Myod1 compared to the tumor-bearing vehicle treated group. The tumor-free and tumor-bearing WFA-treated groups displayed significant increases in both Pax7 and Myod1.

The IRE1 Arm of the UPR is Activated by Withaferin A, but not A2780 Xenografts.

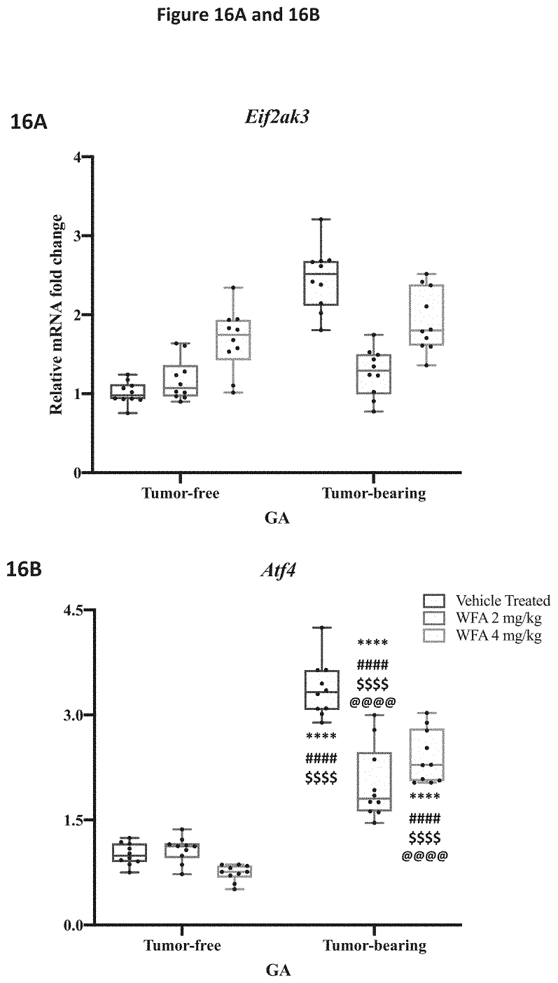

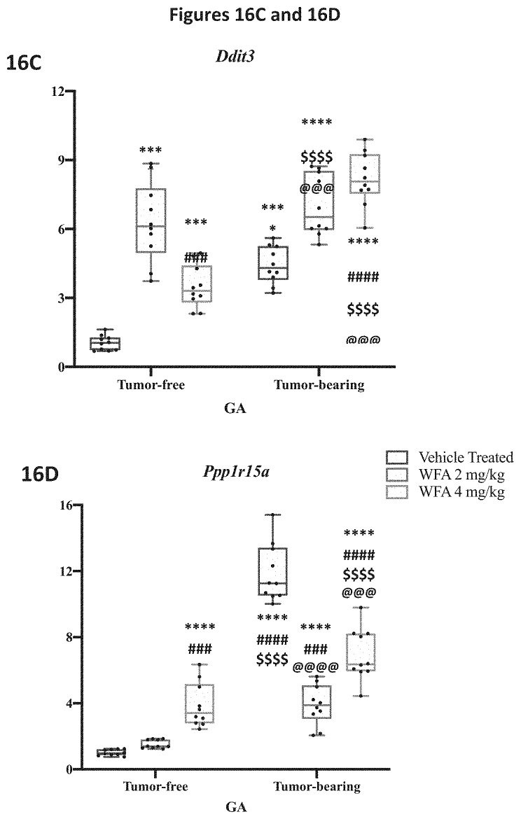

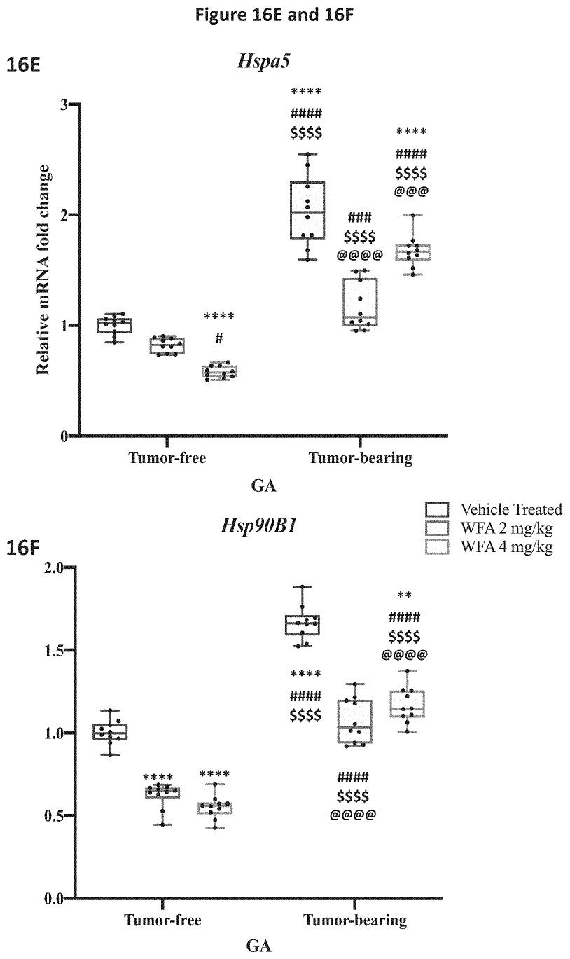

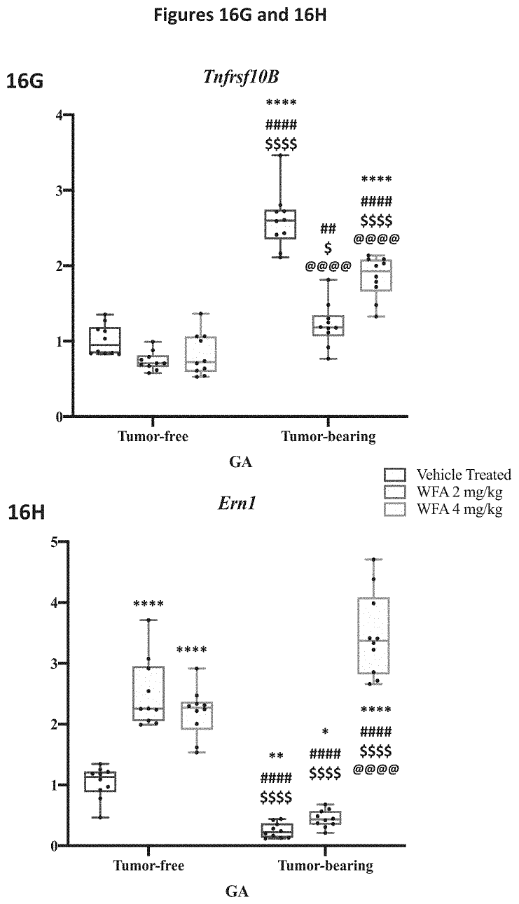

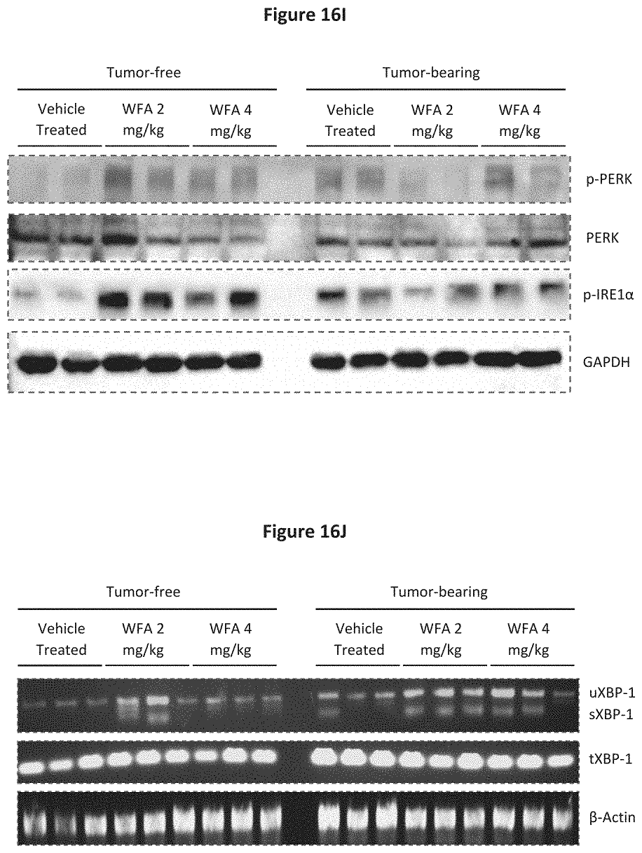

It has been shown in Lewis lung carcinoma and ApcMin/+ models of cancer-induced cachexia that several markers of the unfolded protein response (UPR) are upregulated compared to tumor-free hosts as a byproduct of increased endoplasmic reticulum (ER) stress. Basal levels of UPR activation are necessary for the maintenance of skeletal muscle mass and strength in the context of cancer-induced cachexia. Due to the increased presence of satellite cells and their relationship with the UPR pathways, markers of the UPR were assessed to determine whether they are activated in response to WFA treatment or xenografting of the A2780 ovarian cancer cell line. Globally, both WFA and the xenografted ovarian cancer cell line led to activation of various components of the UPR, with the A2780 ovarian cancer cell line leading to a higher degree of activation than WFA treatment (FIGS. 16A-16H).

The relative transcript levels of Eif2ak3, encoding the protein PERK (FIG. 16A); Atf4, encoding the protein activating transcription factor 4 (ATF4) (FIG. 16B), Ddit3, encoding CCAAT enhancer-binding protein homologous protein (CHOP) (FIG. 16C); Pppr1r15a, encoding growth arrest and DNA damage-inducible protein 34 (GADD34) (FIG. 16D), Hspa5, encoding binding immunoglobulin protein (GRP78 or BiP) (FIG. 16E), Hsp90B1, encoding heat shock protein 90 kDa beta member 1 (GRP94 or HSP90B1) (FIG. 16F), and Tnfrsf10B, encoding death receptor 5 (DR5) (FIG. 16G) were significantly increased in the GA muscle of the tumor-bearing vehicle-treated group compared to the tumor-free vehicle-treated group (p<0.0001 for all comparisons). Surprisingly, relative transcript levels of Ern1, encoding inositol requiring enzyme 1α (IRE1α) (16H) were significantly reduced in the tumor-bearing vehicle-treated group compared to the tumor-free vehicle-treated group (p=0.001). The results are fairly similar to those previously reported by others in an ApcMin/+ model of cancer-induced cachexia, possibly suggesting a commonality in molecular signaling despite the different cellular origins of the tumors.

Observations in the tumor-free mice included: 1) transcriptional upregulation of Eif2ak3, Ddit3, Pppr1r15a, and Ern1, 2) downregulation of Hspa5 and Hsp90B1, and 3) no change in transcript levels of Atf4 and Tnfrsf10B in the WFA-treated groups compared to the vehicle-treated group (p<0.001 or 0.0001 for most comparisons). In the tumor-bearing mice, the WFA treatments produced a mixture of (dis)similar results. Observations in the tumor-bearing WFA 2 mg/kg group included: 1) an upregulation of relative transcript levels of Ddit3 (p<0.01), 2) a reduction in Eif2ak3, Atf4, Pppr1r15a, Hspa5, Hsp90B1, and Tnfrsf10B (p<0.0001 for all comparisons), and 3) no significant difference in relative transcript levels of Ern1 (p=0.9016) compared to the tumor-bearing vehicle-treated group. Observations in the tumor-bearing WFA 4 mg/kg group included: 1) a significant increase in relative transcript levels of Ddit3 and Ern1 (p<0.0001 for both comparisons), and 2) a decrease in relative transcript levels of Eif2ak3, Atf4, Pppr1r15a, Hspa5, Hsp90B1, and Tnfrsf10B (Eif2ak3: p=0.0085; p<0.001 for all other comparisons) compared to the tumor-bearing vehicle-treated group. The expression of Atf6 was not detectable in a sufficient number of samples within the tumor-free or tumor-bearing groups, therefore no conclusions were drawn for this branch of the UPR (data not shown). Markers of the UPR were subsequently assessed by Western blotting to detect protein level changes in ER stress (FIG. 16I). In response to ER stress, IRE1α catalyzes the splicing of XBP-1 mRNA (10). Splicing of XBP-1 was evaluated by performing semi-quantitative RT-PCR using a set of primers that detects both the unspliced and spliced XBP-1 mRNA variants (FIG. 16J). The results from these two experiments corroborate the qPCR findings.

Withaferin A Downregulates the Ubiquitin Proteasome System.

The ubiquitin proteasome system (UPS) is one of the two major proteolytic systems that degrades muscle proteins in oncological settings. As such, relative transcript levels were measured for select E3 ubiquitin ligases that are activated in skeletal muscle in response to catabolic stimuli (41, 42). Fbxo30 (encoding the protein muscle ubiquitin ligase of SCF complex in atrophy-1 (MUSA1)), Fbxo32 (encoding the protein muscle atrophy F-box (MAFbx)), and Trim63 (encoding the protein muscle ring finger 1 (MuRF1)) are muscle-specific E3 ubiquitin ligases that have been shown to be involved in protein degradation under catabolic settings. The E3 ubiquitin ligase Traf6 (encoding the protein Tumor necrosis factor receptor (TNFR)-associated factor 6 (TRAF6)), while not specific to skeletal muscle, is also known to be involved in skeletal muscle atrophy.

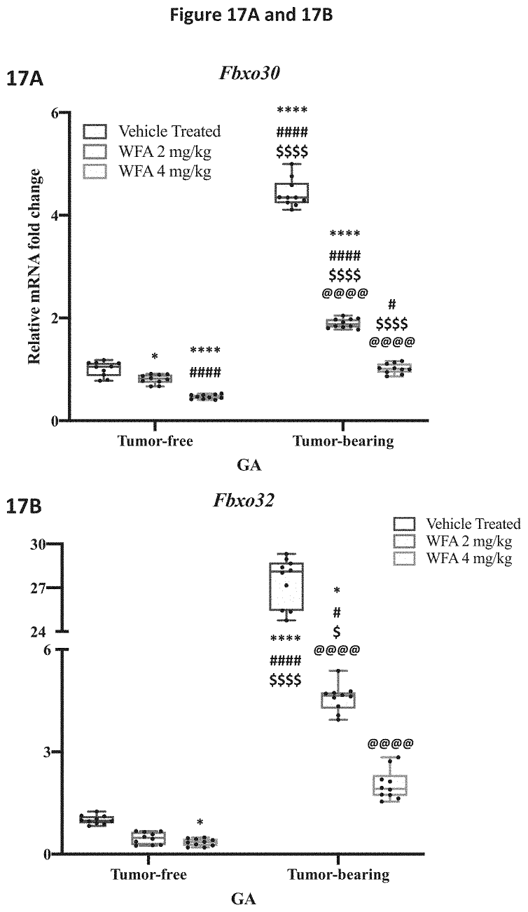

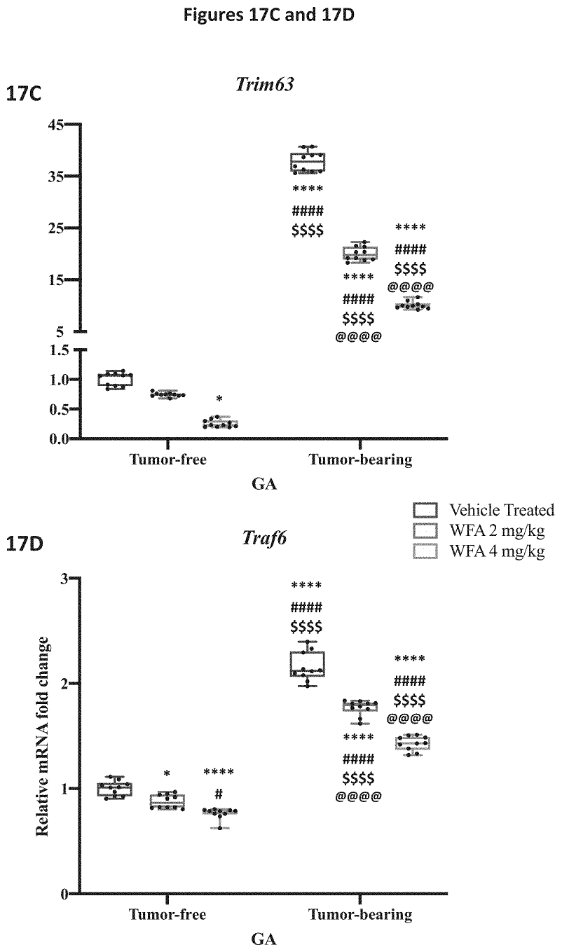

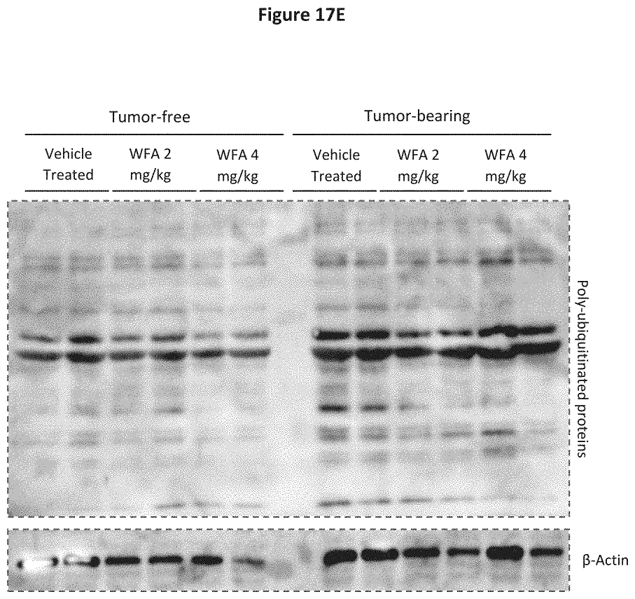

Within the tumor-free groups, there was a significant reduction in the relative transcriptional expression of Fbxo30 and Traf6 in the WFA 2 mg/kg group compared to the vehicle-treated group (p<0.05 for both comparisons) (FIGS. 17A-17D). The WFA 4 mg/kg group exhibited a significant reduction in relative gene expression of Fbxo30, Fbxo32, Trim63, and Traf6 compared to the tumor-free vehicle-treated group (p<0.0001, =0.04, 0.02, <0.0001, respectively). The tumor-bearing vehicle-treated group exhibited a significant upregulation in relative transcript levels of all markers of the UPS assessed compared to all tumor-free groups (p<0.0001 for all comparisons). In the tumor-bearing animals, treatment with WFA led to a statistically significant dose-dependent transcriptional downregulation of Fbxo30, Fbxo32, Trim63, and Traf6 compared to the tumor-bearing vehicle-treated group (p<0.0001 for all comparisons).

To assess the global levels of ubiquitination, immunoblotting against an antibody for ubiquitin was performed, as shown in FIG. 17E. Pan-ubiqutitination of proteins was increased in response to xenografting of the ovarian cancer cells and reduced in response to WFA treatment.

Withaferin A Inhibits the Autophagy Lysosomal System.

In addition to the UPS, cancer-induced cachexia has been shown to activate the autophagy-lysosomal system (ALS), the other major skeletal muscle proteolytic system, to facilitate an atrophying effect on skeletal muscle. During autophagy, LC3B-I is converted to LC3B-II to allow interaction with autophagic vesicles. p62 is a substrate of autophagy that recognizes and binds to ubiquitinated proteins. As such, p62 protein levels decrease with the activation of autophagy and it becomes transcriptionally upregulated to replenish the depleting protein. Beclin1 is another autophagy-related protein that is critical for the initiation of the autophagosome.

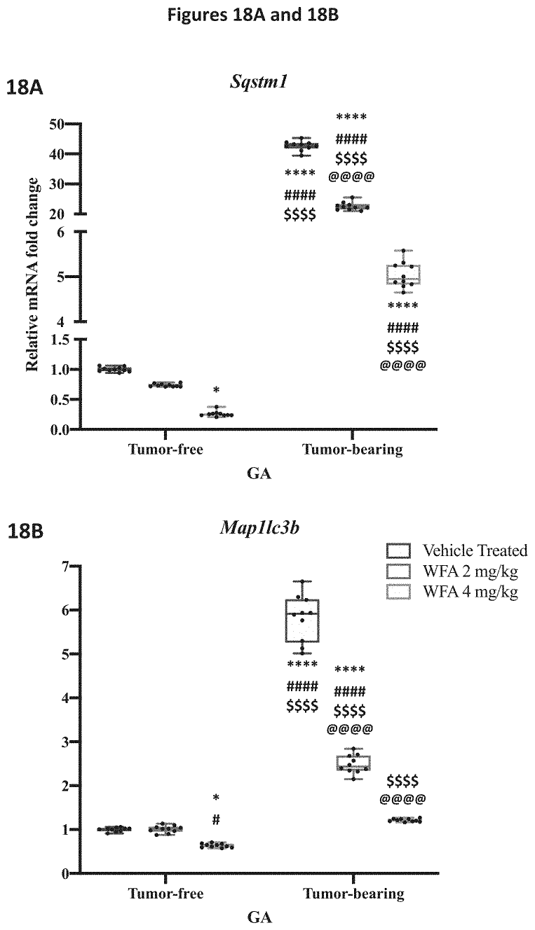

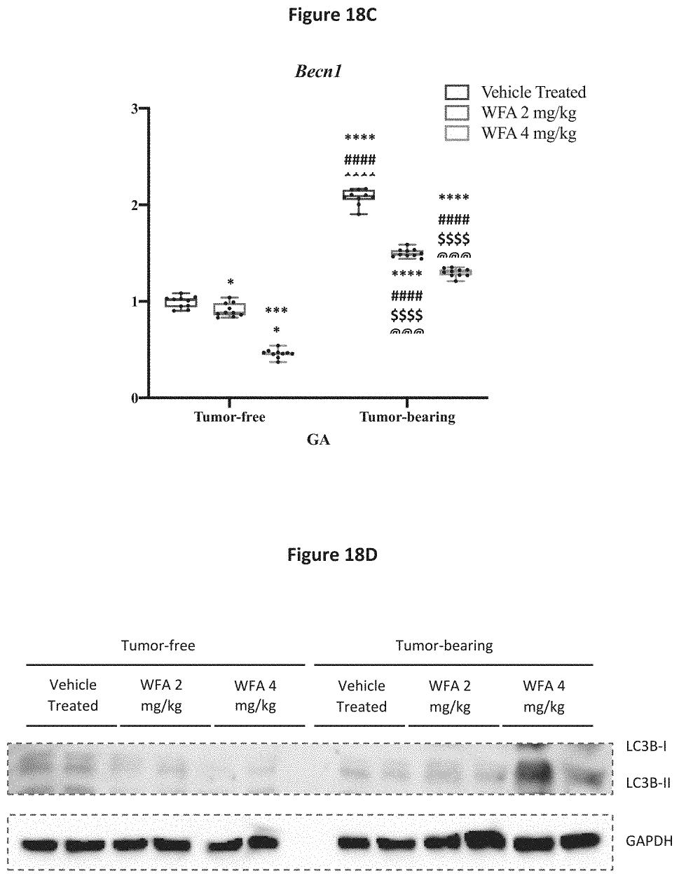

To examine the effect of WFA and the xenograft model on the ALS, relative mRNA levels of common autophagy markers were measured: Sqstm1 (encoding the protein p62), Map11c3b (encoding the protein LC3B), and Becn1 (encoding the protein Beclin1), shown in FIGS. 18A-18C. A significant reduction in relative gene expression of the aforementioned markers of the ALS was observed in the tumor-free WFA 4 mg/kg group compared to the tumor-free vehicle-treated group (p=0.04, 0.013, <0.0001, respectively). In the tumor-bearing vehicle-treated group, we observed a significant increase in relative transcript levels of Sqstm1, Map11c3b, and Becn1 compared to all tumor-free groups (p<0.0001 for all comparisons). Similar to the results for the UPS, WFA treatment led to a significant dose-dependent reduction in relative mRNA expression of the select ALS markers compared to the tumor-bearing vehicle-treated group (p<0.0001 for all comparisons). Work to corroborate the translational changes observed at the protein level are underway (FIG. 18D).

Discussion

WFA treatment led to significant improvements in muscle grip strength, myofibrillar cross-sectional area, and the minimal Feret's diameter in both tumor-free and tumor-bearing mice. The xenograft model of ovarian cancer led to a robust activation of satellite cells without improvements in myofibrillar size or muscle strength in female NSG mice, underlying a common assault to myogenic progenitors. Interestingly, WFA was found to be a more potent activator of satellite cells than the A2780 ovarian cancer xenografts. Whereas the xenografted ovarian cancer cells led to functionally inactive satellite cells (Pax7+/MyoD−), WFA increased the proportion of differentiating satellite cells (Pax7+/MyoD+) potentially explaining the improvements in myofiber CSA and grip strength following WFA treatment. Further, WFA treatment and the xenograft model differentially regulate the UPR pathways in skeletal muscle. WFA appears to produces an adaptive UPR through slight elevation in global UPR activation and robust activation of the IRE1 arm, whereas the A2780 xenografts resulted in muscle atrophy through an activation of the UPS and ALS, due to the induction of a maladaptive UPR. Summarily, WFA is a novel activator of satellite cells that attenuates the effects of cancer-induced cachexia and improves basal muscle strength and myofiber size.

### Example 3

This work was performed in parallel with Example 2 and serves to examine the cardiac component of cachexia. Due to correlation between negative clinical outcomes and alterations in Ang II signaling, the following Example probes the pathway in cardiac tissue in the settings of ovarian cancer. Using the murine model of cancer-induced skeletal muscle cachexia in the Examples of the invention, Example 3 further demonstrates the cancer-induced cardiac atrophy (cachexia) and the therapeutic potential of WFA (WFA). Ovarian tumors were generated by IP injection of 8×10⁵ A2780 ovarian cancer cells instead of 1×10⁶ cells to avoid rapid growth of IP tumors. As in Example 2, the doses of WFA were changed to 2 mg/kg and 4 mg/kg instead of 2 mg/kg and 6 mg/kg because 6 mg/kg WFA turned out to be toxic in previous Examples.

Materials and Methods

Cell Lines.

Cell lines used are as described in Example 1.

Generation of Tumor in Mice.

Experimental tumors were induced as described in Example 2.

Echocardiography.

Left ventricular (LV) systolic and diastolic function were evaluated by transthoracic echocardiography using a Vevo 3100 system and a 40 MHz linear probe (FUJIFILM VisualSonics Inc., Toronto, Ontario, Canada). Mice were anesthetized with isoflurane, maintained at an equivalent surgical depth of anesthesia (induction chamber at 5% with 1.5-2.0 L/min 02 flow, followed by 1.5-2.0% with 1.5-2.0 L/min 02 flow), and placed in the supine position. The skin over the thorax was shaved and then briefly subjected to a depilatory cream. Body temperature was maintained at 37-38° C., and heart rate was monitored using the accompanying Vevo Imaging Station. Briefly, variables that represent diastolic function (E/A ratio, isovolumetric relaxation time (IVRT), and (E/e′) were measured during the resting condition from an apical four-chamber view with conventional pulsed wave tissue Doppler. The E/A ratio was calculated from the peak velocity flow in early diastole (the E wave) to the peak velocity flow in late diastole caused by atrial contraction (the A wave) during resting conditions.

An image was captured along the parasternal short axis and analyzed offline using the Vevo® Lab software. Standard measures of LV structure [i.e., LV internal diameter (LVID) and LV posterior wall thickness (PWT)] and function [i.e., stroke volume (SV), cardiac output (CO), and ejection fraction (EF)] were obtained along the parasternal short axis during resting and stress conditions. In M-mode, wall thickness and chamber dimensions across the sample line were used to calculate anatomical and functional parameters. End-diastolic and end-systolic volumes were estimated from LVID at systole. Results from five cardiac cycles during expiration were averaged together and used for between-group and within-group comparisons. Continuous ECG and heart rate monitoring were performed over a period of one hour while the other measurements were collected.

Histology and Morphometric Heart Analysis.

The hearts were isolated, flash frozen in liquid nitrogen, mounted in O.C.T. embedding medium, and then sectioned using a microtome cryostat. To assess tissue morphology, 8 μm thick transverse sections were cut from the mid-ventricle of the heart. These sections were then subjected to H&E staining or Masson's trichrome staining. Images of H&E-stained and Masson's trichrome-stained heart sections were quantified using Fiji software (National Institutes of Health software) to measure myofiber cross-sectional area (CSA) or the degree of collagen deposition, respectively. Cardiac myofiber CSA was calculated by analyzing 100-200 fibers per heart. Collagen deposition was quantified from the Masson's trichrome-stained tissue.

Imaging.

Slides were prepared and imaged as described in Example 1. Necropsy images were captured on a handheld digital camera and stored as high-resolution JPEG files. Margins of cropped images are indicated by a solid black or white border.

Total RNA Purification and qPCR.

Isolation and preparation of total RNA was performed as described in Example 1. Gene-specific forward and reverse primers used are listed in Table 7.

TABLE 7
Mouse and human gene-specific primers.

| Gene | Species | Direction | Primer Sequence | SEQ ID NO |
|---|---|---|---|---|
| α-MHC | Mus | Forward | 5′-GAGATTTCTCCAACCCAG-3′ | SEQ ID NO: 79 |
| | musculus | Reverse | 5′-TCTGACTTTCGGAGGTACT-3′ | SEQ ID NO: 80 |
| β-MHC | Mus | Forward | 5′-CTACAGGCCTGGGCTTACCT-3′ | SEQ ID NO: 81 |
| | musculus | Reverse | 5′-GCCACAAGCAGGAATGAGAA-3′ | SEQ ID NO: 82 |
| Cardiac | Mus | Forward | 5′-TCTGCCAACTACCGAGCCTAT-3′ | SEQ ID NO: 83 |
| Troponin I | musculus | Reverse | 5′-CTCTTCTGCCTCTCGTTCCAT-3′ | SEQ ID NO: 84 |
| IFNγ | Mus | Forward | 5′-GACAATCAGGCCATCAGCAAC-3′ | SEQ ID NO: 07 |
| | musculus | Reverse | 5′-CGGATGAGCTCATTGAATGCTT-3′ | SEQ ID NO: 08 |
| TNFα | Mus | Forward | 5′-AGCACAGAAAGCATGATCCG-3′ | SEQ ID NO: 03 |
| | musculus | Reverse | 5′-GCCACAAGCAGGAATGAGAA-3′ | SEQ ID NO: 04 |
| IL-6 | Mus | Forward | 5′-CCTTCTTGGGACTGATGCTGG-3′ | SEQ ID NO: 11 |
| | musculus | Reverse | 5′-GCCTCCGACTTGTGAAGTGGT-3′ | SEQ ID NO: 12 |
| MIP-2 | Mus | Forward | 5′-CCACTCTCAAGGGCGGTCAAA-3′ | SEQ ID NO: 15 |
| | musculus | Reverse | 5′-TACGATCCAGGCTTCCCGGGT-3′ | SEQ ID NO: 16 |
| AT1aR | Mus | Forward | 5′-CATTCCTGGATGTGCTG-3′ | SEQ ID NO: 85 |
| | musculus | Reverse | 5′-GAACAAGACGCAGGCTTT-3′ | SEQ ID NO: 86 |
| AT1bR | Mus | Forward | 5′-ATGAATCTCAGAACTCAACAC-3′ | SEQ ID NO: 87 |
| | musculus | Reverse | 5′-AAACTTGAATATTTGGTGGGGA-3′ | SEQ ID NO: 88 |
| β-Actin | Mus | Forward | 5′-CAGGCATTGCTGACAGGATG-3′ | SEQ ID NO: 39 |
| | musculus | Reverse | 5′-TGCTGATCCACATCTGCTGG-3′ | SEQ ID NO: 40 |
| AGT | Homo | Forward | 5′-CTGGCCGCCGAGAAGCTAG-3′ | SEQ ID NO: 89 |
| | sapiens | Reverse | 5′-CCCCACCATGATGGACTGTA-3 | SEQ ID NO: 90 |
| GAPDH | Homo | Forward | 5′-TGATGACATCAAGAAGGTGGT-3′ | SEQ ID NO: 37 |
| | sapiens | Reverse | 5′-TCCTTGGAGGCCATGTGGGCC-3′ | SEQ ID NO: 38 |

Measurement of Plasma Ang

Plasma Ang II levels were measured with an enzyme-linked immunosorbent assay (ELISA). Blood samples were collected in tubes containing ethylenediaminetetraacetic acid (EDTA) (25 mM), o-phenanthroline (0.44 mM), pepstatin A (0.12 mM), and p-hydroxymercuribenzoic acid (1 mM) and then centrifuged at 1200×g for 10 min. The plasma was collected and stored at −80° C. until further processing. Ang II levels were quantified by using a commercially available ELISA kit according to the manufacturer's instructions (Sigma-Aldrich Catalog #MAK190).

Graphical Display and Statistical Analysis.

For the sake of transparency and to display the entire range of data, the majority of the results are expressed as box-and-whisker plots with the box composed of the first, second, and third quartiles and the lower and upper whiskers corresponding to the minimum and maximum values, respectively. Individual data points are depicted as black circles. Image cropping is indicated by a solid black or white line around the field of view. Statistical analysis of the data was performed using one-way analysis of variance (ANOVA) followed by Tukey's honestly significant Difference test (HSDT) post hoc analysis for comparisons between 3 or more groups with one experimental factor or two-way ANOVA followed by Tukey's multiple comparison post hoc test for comparisons between 4 or more groups containing two factors. Statistically significant differences between groups were determined with GraphPad Prism 8.3.0 software for Mac (La Jolla, Calif., USA). ANOVA summaries are presented in the supplementary information. A Tukey-corrected p-value of <0.05 was considered statistically significant, unless otherwise specified.

Results

WFA Ameliorates Gross Body Changes Induced by Ovarian Cancer.

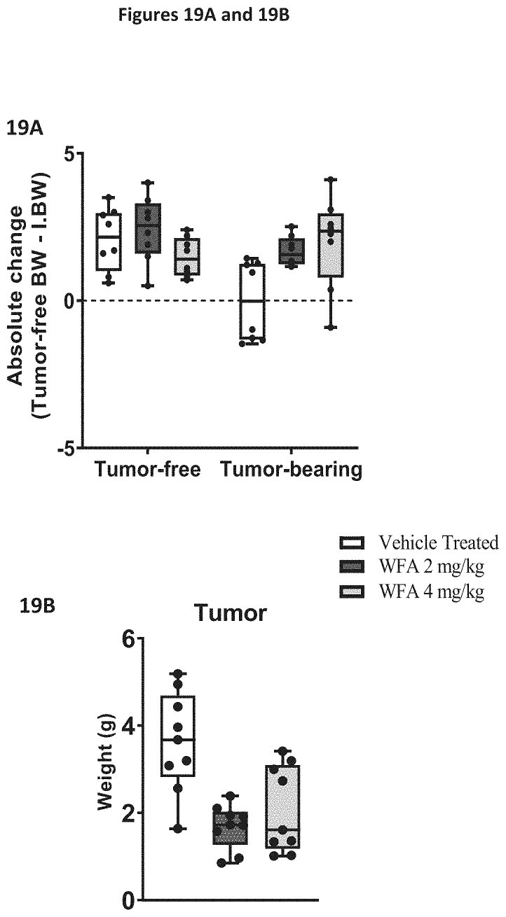

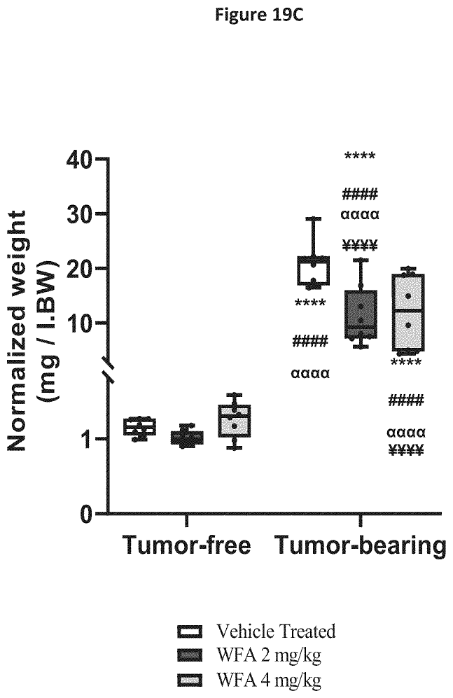

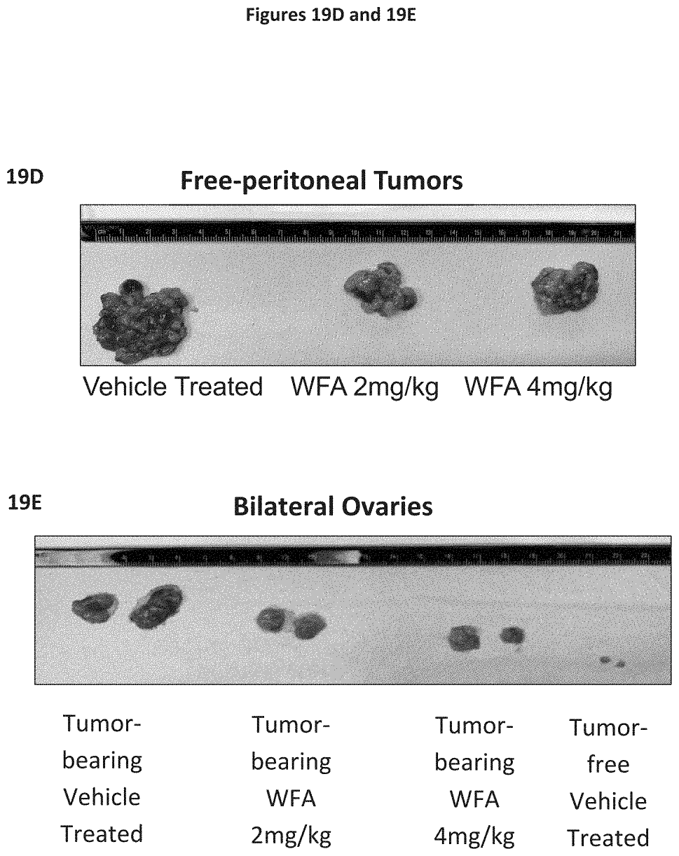

Due to the weight masking effects of ovarian cancer-associated tumor burden, to assess changes in body weight, the absolute change in tumor-free body weight (normalized to the initial body weight (IBW) was calculated. The tumor-bearing vehicle-treated group displayed an average negative change in absolute body weight, shown in FIG. 19A. The remainder of the groups displayed a positive change in absolute body weight. The change in body weight of the tumor-bearing vehicle-treated group was significantly different from the tumor-free vehicle-treated group (p<0.01). Within the tumor-bearing groups, both doses of WFA led to significant preservation of body weight loss compared to that of the tumor-bearing vehicle-treated group (WFA 2 mg/kg: p=0.04; WFA 4 mg/kg: p=0.009), but the differences were not significantly different from those of the tumor-free groups (p>0.90). WFA treatment significantly reduced the weight of visible tumors (WFA 2 mg/kg: 1.6±0.19 g; WFA 4 mg/kg: 1.9±0.35 g) compared to that of the vehicle-treated group (3.6±0.43 g) (WFA 2 mg/kg: p=0.0004; WFA 4 mg/kg: p=0.004), shown in FIGS. 19B and 19C. No significant difference in tumor weight between the two doses of WFA was observed (p=0.65).

The IP tumors were metastasized to select organs/anatomic regions known to be highly estrogen-responsive, including free-peritoneal tumors shown in FIG. 19D and bilateral ovaries shown in FIG. 19E. The normalized weights of the bilateral ovaries were found to be significantly increased in all three tumor-bearing groups compared to all three tumor-free groups, as a byproduct of tumor metastasis (21.9±1.3 mg in the tumor-bearing vehicle-treated group compared to 1.13±0.04 mg/g IBW in the tumor-free vehicle-treated group) (p<0.0001 for all comparisons), as was shown in FIG. 19C. However, both WFA treatments led to a significant reduction in the normalized weights of the bilateral ovaries in the tumor-bearing groups (WFA 2 mg/kg: 10.38±1.32 mg/g IBW; WFA 4 mg/kg: 12.05±2.09 mg/g IBW) compared to those of the tumor-bearing vehicle-treated group (WFA 2 mg/kg: p<0.0001; WFA 4 mg/kg: p=0.0002).

No significant differences were found in the normalized weights of other common metastatic sites of ovarian cancer, such as the brain, lungs, liver, or kidney. This could be attributable to differences in the metastatic model utilized versus metastatic routes that could be exhibited in a primary model of ovarian cancer. While a thorough histological examination of all peritoneal/retroperitoneal organs was not performed, at a gross level the majority of the metastatic lesions encapsulated the organs, such as the spleen.

WFA Preserves Systolic Function.

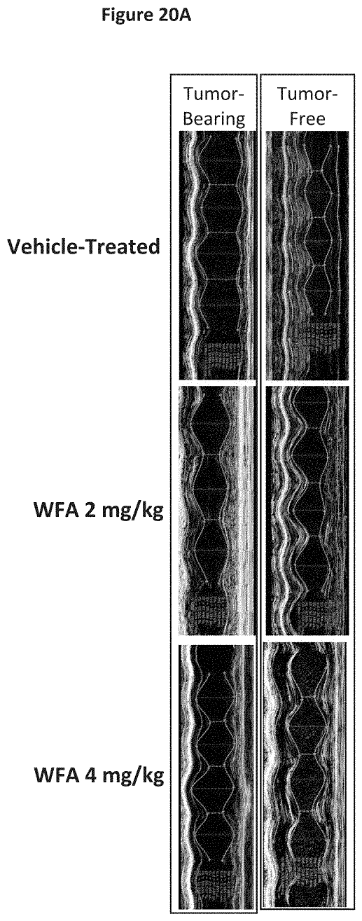

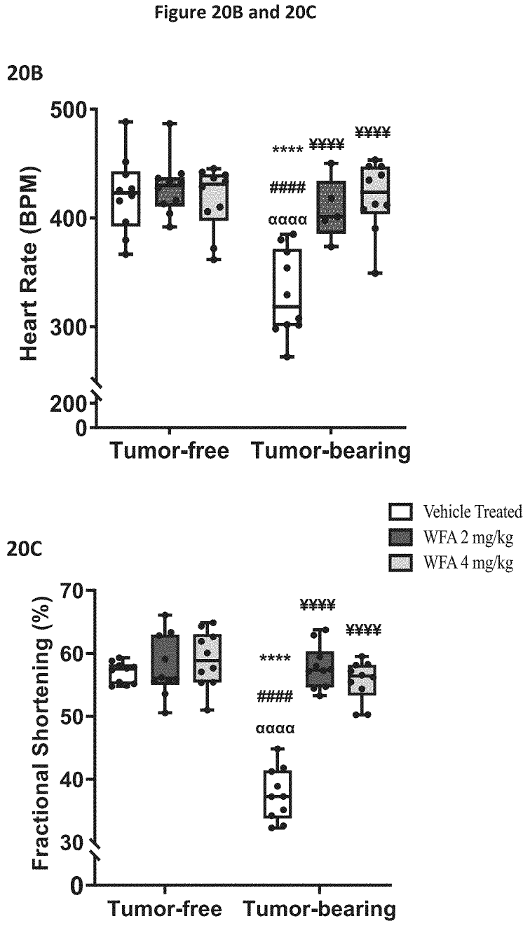

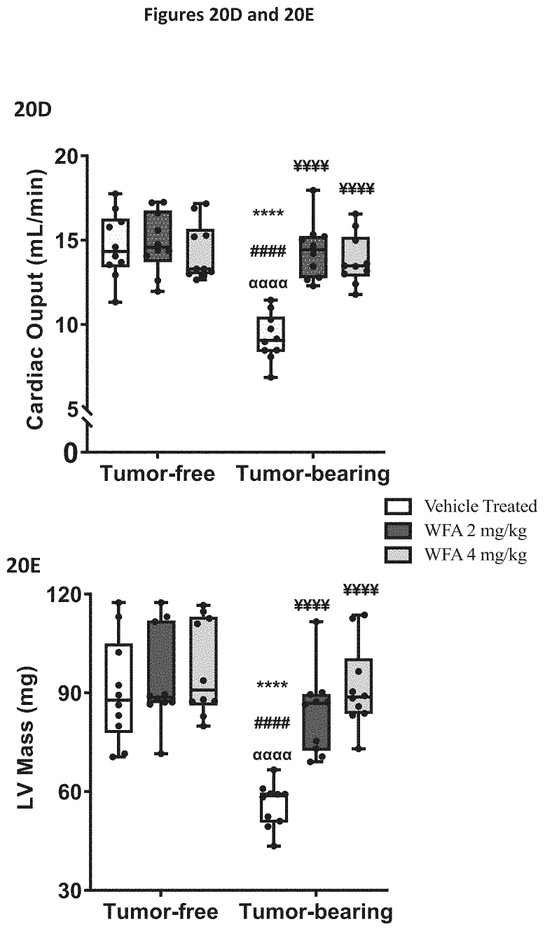

While skeletal muscle is primarily thought to be the primary target of cancer-induced cachexia, cardiac muscle is also affected to varying degrees by cancer, resulting in cardiac cachexia. To investigate whether ovarian cancer alters the contractile functions of the heart, echocardiography was performed prior to euthanization. Representative M-mode traces for all the groups are shown in FIG. 20A. Echocardiographic assessment revealed that the tumor-bearing vehicle-treated group displayed a significant reduction in heart rate compared to the tumor-free groups (p<0.0001 for all comparisons), as shown in FIG. 20B, likely due to persistent arrhythmias that were detected during the entire duration of the echocardiographic assessment. These arrhythmias (variable AV blocks) were only detectable in the tumor-bearing vehicle-treated group. Due to experimental limitations, continuous monitoring of heart activity via implantation of a DSI device was not possible due to the fragile health state of this group. Thus, a definitive diagnosis could not be made with respect to the arrhythmias. No significant differences in heart rate within the tumor-free or tumor-bearing WFA-treated groups were observed compared to those of the tumor-free vehicle-treated group.

As a first step toward assessing LV systolic function, the echocardiographically determined percentage of fractional shortening (FS) was measured and compared, shown in FIG. 20C. FS was significantly reduced in the tumor-bearing vehicle-treated group (35.0±3.2% compared to 58.0±2.2% in the tumor-free vehicle-treated group) (p<0.0001 for all comparisons). The reduction in FS was completely rescued by treatment with WFA at both doses in the tumor-bearing groups compared to that of the tumor-bearing vehicle-treated group (FS: WFA 2 mg/kg=57.8±3.6%; WFA 4 mg/kg=55.3±3.2%; p<0.0001 for both comparisons).

Similar changes were observed in cardiac output (CO), which is heart rate- and stroke volume-dependent, shown in FIG. 20D. Due to the reduction in heart rate in the tumor-bearing vehicle-treated group, a concomitant decrease in CO was observed. Along similar lines, because WFA treatment normalized the heart rates of tumor-bearing mice, the CO was similarly normalized. The trends and levels of significance for the differences in CO mirrored the observed changes in heart rate. WFA treatment did not have any effect on HR or CO in tumor-free mice.

One of the potential applications of echocardiography in mice is to noninvasively assess LV mass in experimental animals. The LV mass of all tumor-free and tumor-bearing animals was calculated and is shown in FIG. 20E. No significant difference in LV mass was noted between the tumor-free groups that were treated with vehicle or with either dose of WFA (p>0.80 for both comparisons). However, a significant reduction in LV mass was observed in the tumor-bearing vehicle-treated group (55.6±2.3 mg compared to 91.8±5.5 mg in the tumor-free vehicle-treated group (p<0.0001 for all comparisons). Treatment of tumor-bearing mice with WFA led to a significant increase in LV mass compared to that of the tumor-bearing vehicle-treated group (WFA 2 mg/kg: p=0.0002; WFA 4 mg/kg: p<0.0001). The reduction in LV mass in the tumor-bearing vehicle-treated group could be attributed to cancer-induced atrophy of cardiomyocytes. Changes in LV mass in tumor-free mice treated with WFA were not observed.

Previous studies have shown cardiac dysfunction and atrophy in cancer models described sex-specific differences in the progression of cardiac cachexia. In a mouse model of colon adenocarcinoma, male mice lost significantly more cardiac mass than females (see Cosper et al. Cancer Res 2011; 71(5):1710-1720). In these Examples, the robust cachectic phenotype more closely resembled the loss of cardiac mass in the male mice of the colon-adenocarcinoma model. These results are the first indication that generation of ovarian tumor in mice results in a significant reduction in cardiac function and mass.

WFA Alleviates Ovarian Cancer-Induced Diastolic Dysfunction.

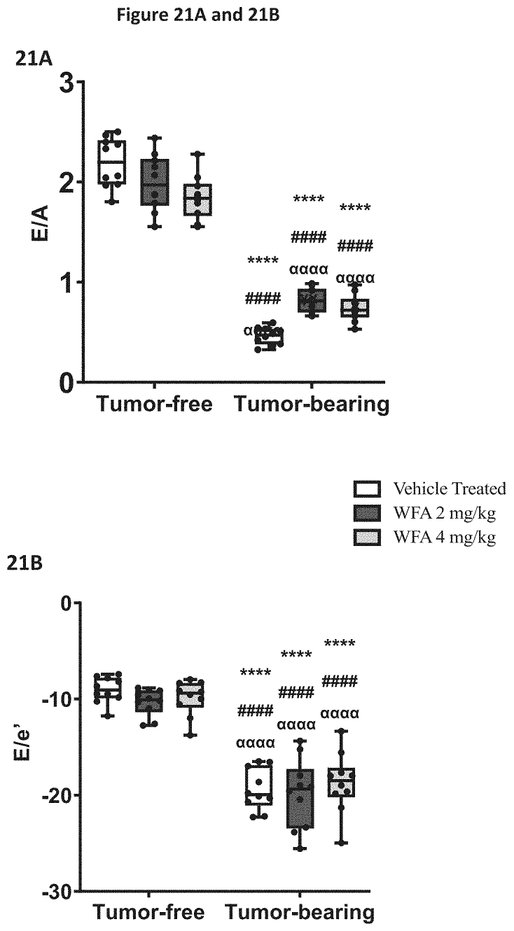

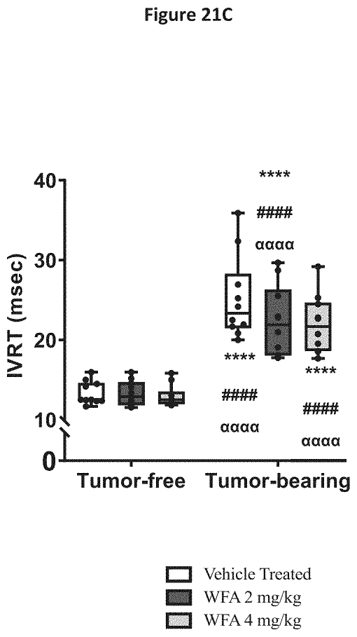

Some studies have shown that under certain pathological paradigms, systolic function is spared, whereas a dysfunctional state is evidenced in the diastolic phase (see Schnelle et al. J Mol Cell Cardiol. 2018; 114:20-28). Therefore, we next investigated whether ovarian cancer induced diastolic dysfunction. Two common measures of diastolic function, the E/A ratio and the E/e′ ratio, were significantly lower in the tumor-bearing vehicle-treated group (E/A: 0.45±0.12 compared to 2.17±0.05 in the tumor-free vehicle-treated group; E/e′: −18.6±0.6 compared to −9.5±0.65 in the tumor-free vehicle-treated group) than in the tumor-free groups (p<0.0001 for all comparisons), shown in FIGS. 21A and 21B. While WFA treatment significantly attenuated systolic dysfunction, diastolic dysfunction was partially improved in the tumor-bearing WFA-treated groups compared to that of the tumor-bearing vehicle-treated group (WFA 2 mg/kg: p=0.002; WFA 4 mg/kg: p=0.03), but was still significantly reduced compared to the tumor-free groups (p<0.0001 for all comparisons). Another common diastolic parameter, the isovolumetric relaxation time (IVRT), was significantly prolonged in the tumor-bearing groups compared to those of the tumor-free groups (with no significant differences within the tumor-free or tumor-bearing groups), as shown in FIG. 21C. WFA did not affect diastolic function in tumor-free animals. Taken together, these results demonstrate that ovarian cancer induces diastolic dysfunction in the heart (i.e., an impairment in relaxation). Further, while WFA completely preserved systolic function, it did not completely reverse diastolic dysfunction. This suggests that a longer treatment with WFA may be necessary to completely resolve the dysfunctional state of the heart induced by cancer.

WFA Attenuates Cardiac Cachexia.

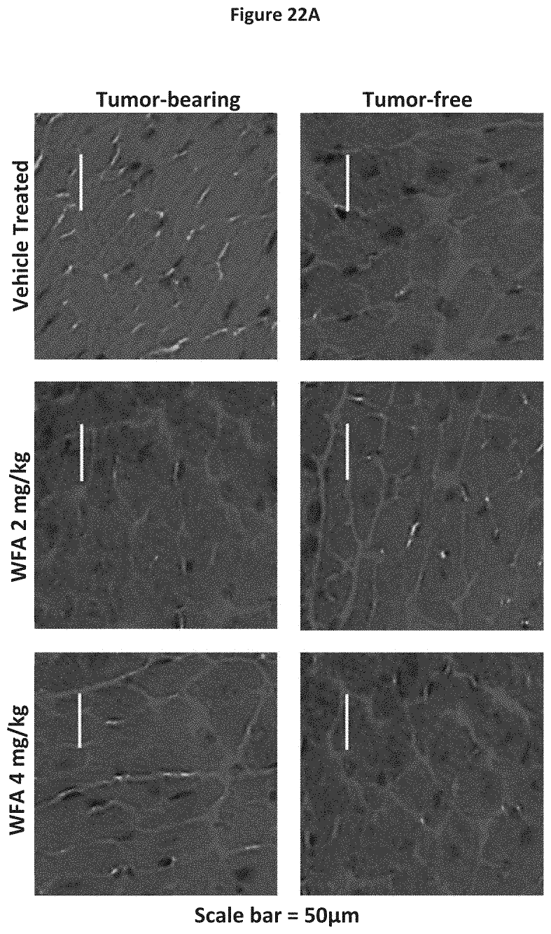

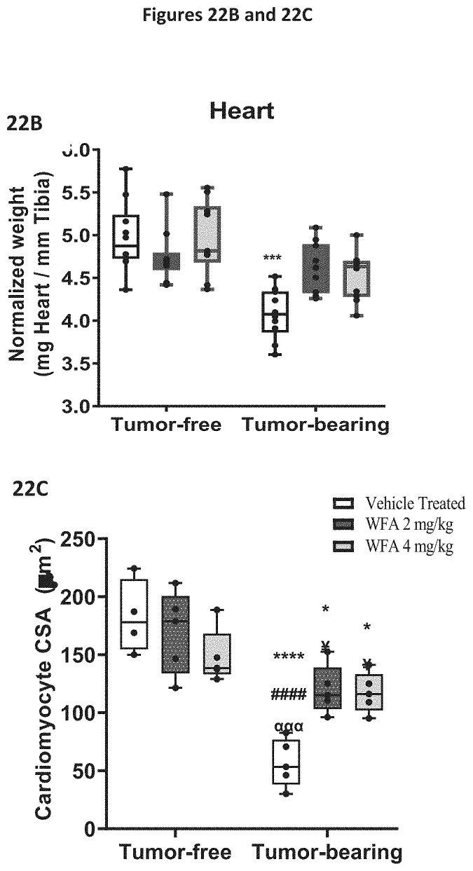

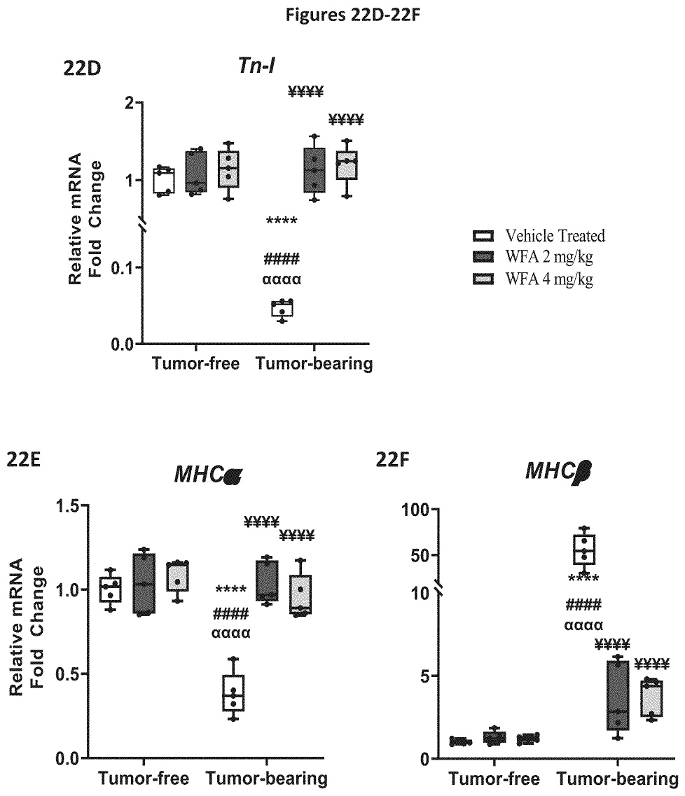

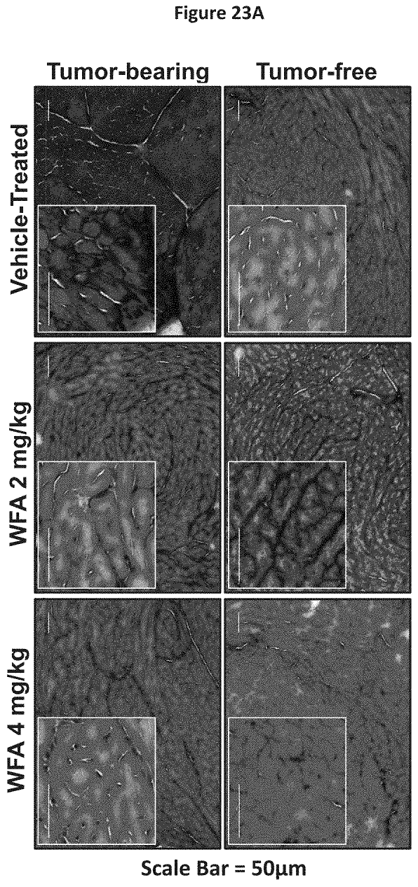

Morphological changes in the heart, such as atrophy or hypertrophy of cardiomyocytes and collagen deposition/fibrotic scarring are known to cause cardiac dysfunction. Based upon the systolic and diastolic changes associated with the ovarian cancer model and WFA treatment, changes in the heart at the gross anatomical levels were examined, as shown in FIG. 22A. Xenografting of ovarian cancer cells resulted in a reduction in heart weight (normalized by tibial length) in the tumor-bearing vehicle-treated group compared to tumor-free groups (p<0.0001 for all comparisons). This effect was significantly blunted upon treatment with both doses of WFA in tumor-bearing mice compared to the tumor-bearing vehicle-treated group (p<0.001 for both comparisons). WFA did not have any effect on heart weight in tumor-free animals, shown in FIG. 22B. We next examined changes in the heart at a morphometric level using H&E staining of mid-ventricular sections of the heart. Similar to the findings in skeletal muscle in our previous study, we observed a significant decrease in the cross-sectional area (CSA) of cardiomyocytes in the tumor-bearing groups compared to those of the tumor-free groups, as shown in FIG. 22C. WFA treatment resulted in a partial recovery of the CSA of cardiomyocytes in the tumor-bearing WFA-treated groups compared to those of the tumor-bearing vehicle-treated group (p<0.0001 for all comparisons) and the tumor-free vehicle-treated group (p=0.01), but there was no difference between tumor-free animals treated with WFA.

In addition to changes at the morphometric level, we observed a significant reduction in relative transcript levels of Troponin-I (Tn-I; a major contractile protein) in the tumor-bearing vehicle-treated group compared to those of the tumor-free groups (p<0.0001 for all comparisons), shown in FIG. 22D. This effect was significantly blunted upon treatment with both doses of WFA in tumor-bearing mice compared to the tumor-bearing vehicle-treated group (p<0.0001 for both comparisons). We also observed a significant shift in MHC isoforms from a predominantly adult α-MHC state to one that is primarily embryonic β-MHC in the tumor-bearing vehicle-treated group compared to the tumor-free vehicle-treated group, shown in FIGS. 22E and 22F. The changes in motor proteins corroborates the functional changes observed in response to the tumor burden.

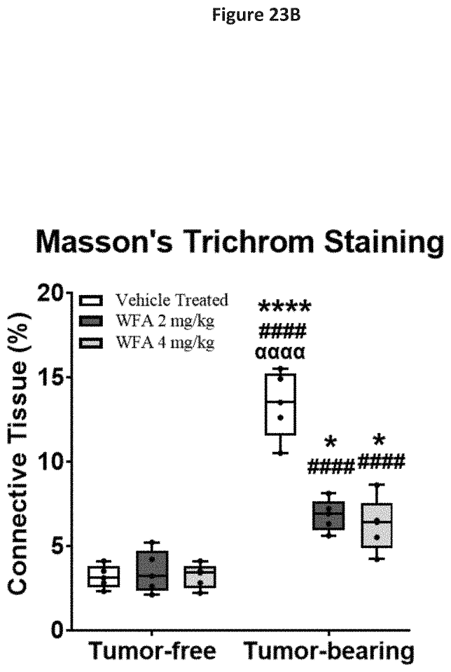

In addition to atrophy, some models of cancer-induced cachexia result in fibrotic changes in muscle. Therefore, sections from the mid-ventricle of the heart were subjected to Masson's trichrome staining to elucidate collagen deposition. A basal degree of blue-stained collagen was clearly noted in the tumor-free vehicle-treated group as shown in FIG. 23A, but as expected, this collagen deposition was intimately associated with the vasculature. A comparable amount of connective tissue was observed in the tumor-free WFA-treated groups compared to that of the tumor-free vehicle-treated group (p>0.90 for both comparisons), as shown in FIG. 23B. All the tumor-bearing groups exhibited an increase in fibrosis compared to the tumor-free groups. The percentage of connective tissue was significantly decreased in the tumor-bearing mice that were treated with either dose of WFA compared to the tumor-bearing vehicle-treated group (p<0.01 for both comparisons), but was significantly higher compared to tumor-free groups (p<0.001 for all comparisons), suggesting WFA partially rescues the heart from excessive connective tissue formation. The atrophy and fibrotic changes in the heart are known to result in cardiac dysfunction. Based on heart dysfunction and morphometric changes in cardiac muscle (cardiomyocytes) induced by ovarian cancer, the results in this Example clearly demonstrate the induction of cardiac cachexia by ovarian cancer and its reversal by WFA.

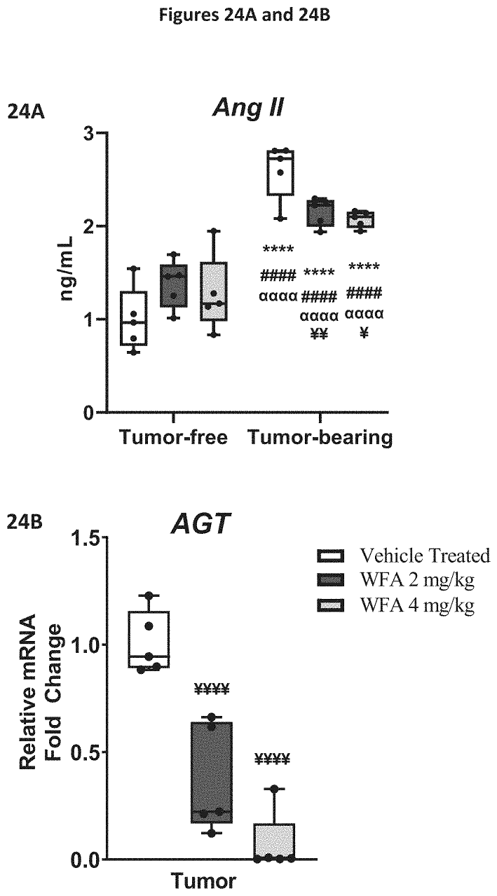

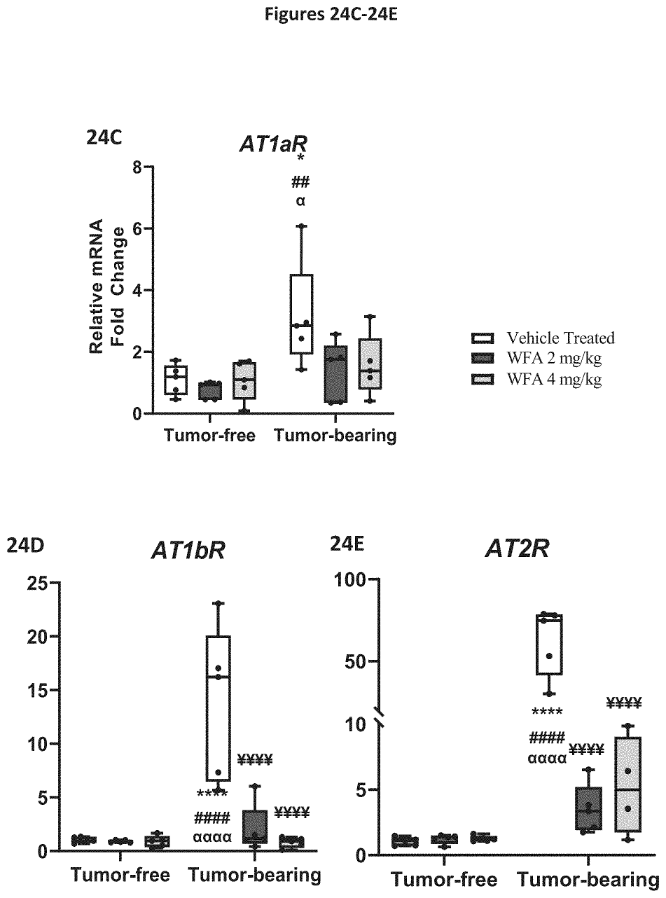

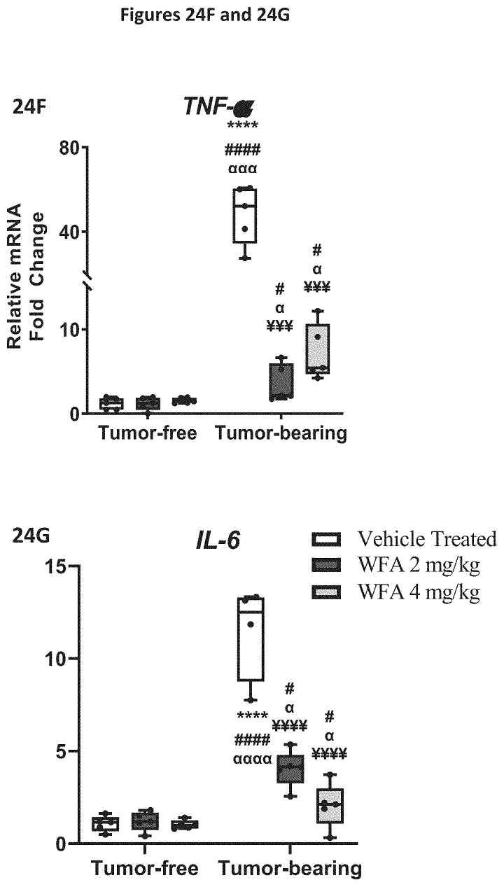

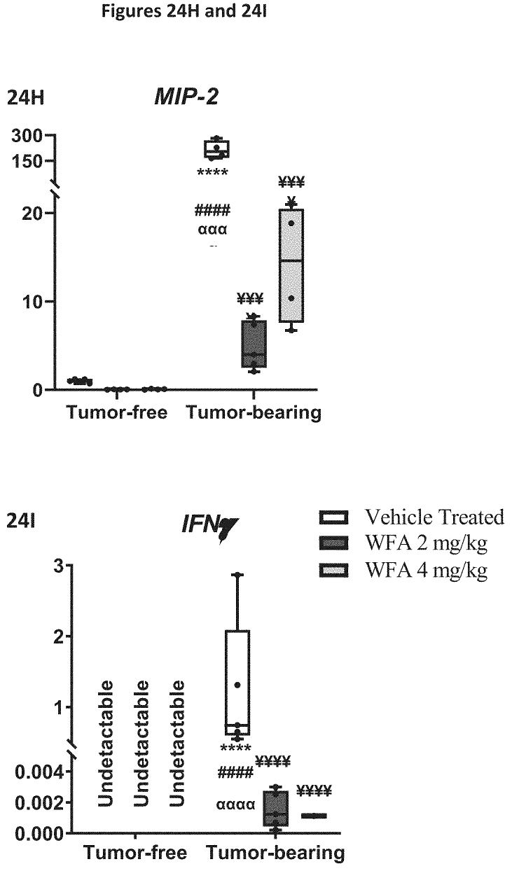

WFA decreases levels of circulating angiotensin II and proinflammatory markers: A few studies have shown that increased levels of circulating Ang II induce myocardial damage, which can lead to cardiac cachexia. Studies have also shown that the increase in circulating levels of Ang II are most likely secreted from cancer cells (see Xie et al. J Immunother Cancer. 2018; 6(1):88). To corroborate these findings, Ang II levels in plasma fractionated from centrally collected blood were measured, as shown in FIG. 24A. Plasma Ang II levels were found to be significantly higher in the tumor-bearing vehicle-treated group compared to the tumor-free vehicle-treated mice (2.7±0.3 ng/ml compared to 1.00±0.34 ng/ml, p<0.0001). WFA treatment significantly reduced the circulating levels of Ang II in the tumor bearing mice compared to the tumor-bearing vehicle-treated group (WFA 2 mg/kg: 2.15±0.015 ng/ml, p<0.01; WFA 4 mg/kg: 2.07±0.08 ng/ml, p<0.01). In addition, we measured the relative transcript levels of angiotensinogen in tumors from the tumor-bearing mice, shown in FIG. 24B. Treatment with WFA significantly reduced relative transcript levels of angiotensinogen in tumor samples collected from mice (WFA 2 mg/kg: 0.3676±0.252 fold change; WFA 4 mg/kg: 0.0694±0.144 fold change compared to 1.0081±0.144 fold change in the vehicle-treated group, p<0.0001).

Ovarian cancer prognosis, tumor angiogenesis, and patient outcome are correlated with angiotensin II receptor 1 and 2 levels (AT₁R, AT₂R). Humans have one isoform of AT₁R, whereas mice have two subtypes, AT₁ₐR and AT_{1b}R. Thus, relative transcript levels of AT₁ₐR, AT_{1b}R, and AT₂R in the heart in all of the tumor-free and tumor-bearing groups were measured (FIGS. 24C-24E). A significant increase was observed in AT1aR transcription in cardiac tissues of tumor-bearing vehicle-treated mice (3.14±1.7 fold change compared to tumor-free vehicle-treated mice, p<0.01) (FIG. 24C), as well as for AT/bR (13.86±7.2 fold change, p<0.0001) (FIG. 24D). There was a 62.89±21.13 (p<0.0001) fold increase of AT₂R in tumor-bearing animals compared to control, which was significantly reduced in WFA treated tumor-bearing animals (WFA 2 mg/kg: 3.5±1.8, WFA 4 mg/kg: 5.2±3.2), shown in FIG. 20E.

The mechanisms of Ang II-induced muscle atrophy are complex and include potential direct effects of inflammatory cytokines such as IL-6, TNF-α, and IFN-γ. Therefore, we assessed the expression of NF-κB-related pro-inflammatory cytokines in the heart myocytes. As shown in FIGS. 24F-24I, the relative transcript levels of all proinflammatory cytokines assessed (TNFα, IL-6, MIP-2 (the mouse ortholog of human IL-8), and IFNγ), were significantly increased in the tumor-bearing vehicle-treated group and were significantly reduced in the WFA-treated groups compared to the tumor-free vehicle-treated group (p<0.0001) (FIG. 5D). The relative mRNA levels of IFNγ were found to be undetectable in tumor-free animals, so this component is normalized to the tumor-bearing vehicle-treated group to allow a partial analysis, as shown in FIG. 24I. The transcript levels of IFNγ were detectable in the tumor-bearing vehicle-treated mice and were significantly reduced in the WFA-treated groups.

Discussion

Involuntary weight loss and a reduction in skeletal muscle mass remain the focus of cachexia research. Recent studies have also provided evidence that cancer-induced cachexia also causes significant cardiac atrophy, resulting in a state of cardiac cachexia and contributing to chronic heart failure. However, there was previously no information showing that ovarian cancer induces cardiac cachexia and no effective drug is available to treat it. The Examples of the invention establish the cardiac cachexia phenotype and the use of WFA against cancer-induced cardiac cachexia.

Most common models of cancer-induced cachexia are the C26 colon cancer, Lewis lung carcinoma, and Apc^{min/+} models. Some of these models of cancer-induced cachexia demonstrate varying degrees of concomitant cardiac atrophy. A significant reduction in heart weight and cardiac dysfunction were observed in male mice; however, this was significantly diminished in female mice. Indeed, most studies on cachexia exclude the utilization of female lab animals, likely due to the more prevalent phenotype exhibited in males. As such, there is a fundamental lack of knowledge about not only the skeletal muscle effects, but also the cardiac effects of cancer-induced cachexia in female lab animals.

The preceding Examples characterize the cachectic effects associated with in vivo growth of A2780 ovarian cancer xenografts at a functional level with respect to both cardiac and skeletal muscle. Gross body changes and effects on skeletal muscle atrophy associated with tumor burden confirming validate the cachectic phenotype, suggesting that female lab animals provide a suitable model for both skeletal muscle and cardiac cachexia. Extensive studies have addressed the mechanisms underlying the atrophying effects of cancer on skeletal muscle, but the effects on cardiac muscle were less understood prior to now.

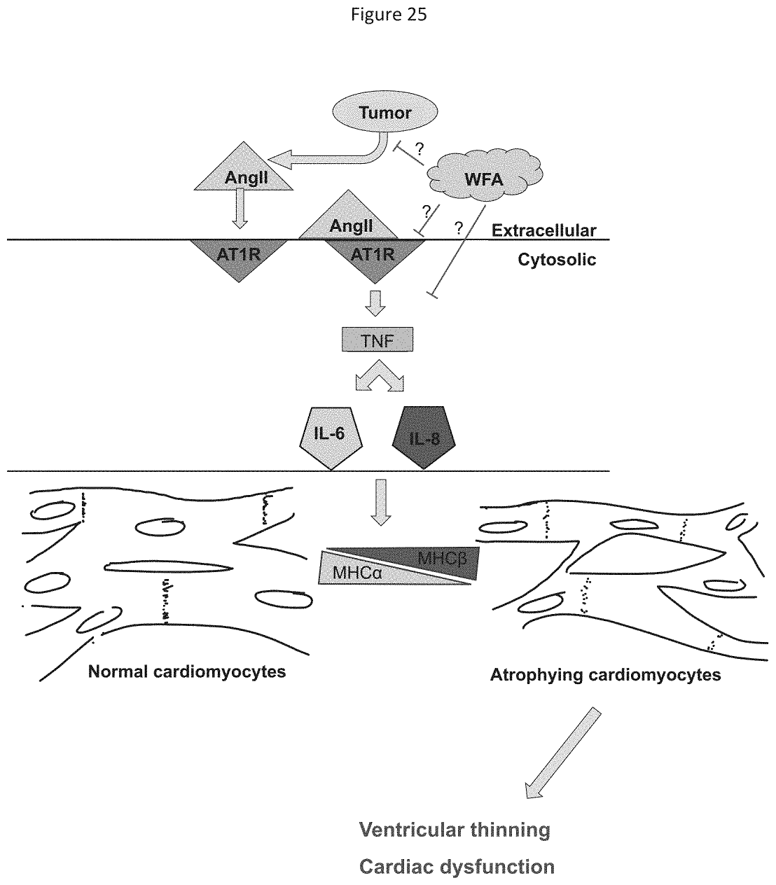

With respect to skeletal muscle, cachexia causes hypercatabolism of sarcomeric motor proteins. Without being bound by theory, this impairment in skeletal muscle contraction facilitates atrophy. Cancer induces a shift in metabolism to a predominantly glycolytic state. Skeletal muscle myofibers in turn undergo oxidative to glycolytic myofiber-type conversions. This is mediated by upregulation of embryonic myosin heavy chain (MHC) proteins and downregulation of adult MHCs. Interestingly, cardiac failure is also characterized by switching gene expression from adult to fetal MHC isoforms, potentially serving as surrogate markers for the induction of cardiac cachexia (FIG. 25). MHCα, which is predominant in adult mouse hearts, has higher ATPase activity than MHCβ, which is predominant during embryonic development. Even a small magnitude of isoform shift could significantly impact heart function. The qPCR results in the preceding Examples demonstrate a significant reduction in relative transcript levels of MHCα and a significant increase in MHCβ in the tumor-bearing groups, which might be an adaptive response of the heart to conserve energy that contributes to the damaged contractile function in tumor-bearing mice. Additionally, cardiac troponin-T and troponin-I are regulatory proteins that control the calcium-mediated interaction between myosin and actin. Previous studies have shown that elevated levels of troponin-T are contingent upon the death of cardiomyocytes. Studies evaluating cardiac cachexia have not shown much of an effect on troponin-T. However, in the context of cardiac cachexia, troponin-I (a key myofilament protein) is altered in response to changes in the contractility of the heart, as shown in Example 3. The Example demonstrates tumor-induced cardiac remodeling and myocardial dysfunction which are consistent with other studies focused on C26 colon cancer in males.

Many studies have demonstrated effect of WFA on apoptosis of cardiomyocytes (see Guo et al. Circ J. 2019; 83(8):1726-36). The Examples have shown that WFA attenuated skeletal muscle cachexia, and microscopic analysis of the hearts of tumor-bearing mice showed enhanced intermyofibrillar collagen deposition and cardiomyocyte atrophy. Indeed, a significant degree of fibrosis was evidenced in the heart in the tumor-bearing vehicle treated group. Approximately 20% of the area within the heart was deeply stained for collagen, similar to what has been observed in colon cancer models of cachexia. Fibrosis of cardiac tissue commonly occurs in myocardial pathologies and is known to cause a decrease in the performance of the heart. This collagen deposition causes stiffening of the heart, which in turn leads to worsening of heart failure, primarily via diastolic dysfunction. Interestingly, due to an absence of noticeable symptoms, diastolic dysfunction is relatively underdiagnosed compared to systolic dysfunction. Both systolic and diastolic dysfunction was observed in the hearts of tumor-bearing mice in the Examples. WFA preserved systolic function and partially improved diastolic function, which could be attributed to the decrease in fibrous scarring in the heart.

WFA has an anti-inflammatory ability by direct inhibition of NF-κB signaling on skeletal muscle. WFA ameliorated the cachectic phenotype exhibited in ovarian cancer. Tumor-induced cardiac remodeling involves increased levels of pro-inflammatory cytokines, such as IL-1β, IL-6, and IL-8, as well as ventricular thinning and decreased troponin-I levels, resulting in dysfunctional contraction and relaxation of the heart. The concomitant induction of skeletal muscle and cardiac muscle cachectic phenotypes suggest the presence of a similar mechanism of induction. Nevertheless, WFA treatment promisingly attenuated or completely rescued the effects induced by the xenografted ovarian cancer.

TNFα is a pro-inflammatory cytokine with a wide range of biological effects that has been implicated in the pathophysiology of many cardiovascular diseases. TNFα is central in initiating and sustaining the pro-inflammatory cytokine cascade, and simulates the production of other cytokines, such as IL-6 and IL-8. TNFα induces myocardial fibrosis by inhibiting phagocytosis of collagen in the heart. The qPCR data in the Examples demonstrated a drastic increase in TNFα transcription with a concomitant increase in fibrosis in tumor-bearing mice, which was reduced by WFA treatment. WFA is known to inhibit activation of the NF-κB pathway, resulting in a downregulation of pro-inflammatory cytokines, such as TNFα and IL-6. TNFα, IL-6, IL-8, IL-1β, and IFNγ are considered to be the major inflammatory mediators of cancer-induced cachexia, which have been demonstrated to be elevated in various animal models of cancer. The qPCR data showed that local inflammation was increased in the hearts of tumor-bearing mice, as demonstrated by the increase in relative transcript levels of IL-6 and MIP-2 transcription. TNFα contributes to Ang II-induced adverse cardiac remodeling. Ang II importantly contributes to the induction of skeletal muscle wasting, which is partially mediated by AT₁R. In skeletal muscle, it was demonstrated that an AT₁R inhibitor was sufficient in attenuating the induction of a cachectic phenotype. Studies have shown that overexpression/activation of AT₂R attenuated ischemia-induced cardiac remodeling. However, controversial findings are reported recently. Overexpressing AT₂R specifically in ventricular cardiomyocytes decreased cardiac contractility and dilated cardiomyopathy, while severity of cardiac dysfunction was positively correlated with level of AT₂R expression. The level of overexpression determines beneficial or detrimental role of AT₂R to the heart: low or moderate levels of overexpression of AT₂R protects the heart against ischemia-induced injury, meanwhile this protection is inversely related to the overexpression levels of AT₂R. In addition to the effect on skeletal muscle, the Examples show that AT₁R and AT₂R inhibition can be beneficial for cardiac muscle.

The Examples of xenograft model of ovarian cancer show that with the proper oncological paradigm, female lab animals are suitable models of human cachexia. The Examples underscore the intersection between cancer and the heart. The cardiac cachectic phenotype in this model was rescued by treatment with WFA. WFA treatment attenuates plasma Ang II levels in tumor-bearing mice, and pro-inflammatory markers induced through AT₁R in tumor-bearing mice were abrogated by WFA treatment. WFA can reverse heart dysfunction in established cardiac cachexia and inhibit the development of cardiac cachexia.

While the invention has been described in its preferred embodiments, those of skill in the art will recognize the invention can be practiced with variations within the spirit and scope of the appended claims.

We claim:

1. A method of ameliorating cachexia in a subject in need thereof, comprising administering to the subject a therapeutically effective amount of a steroidal lactone.

2. The method of claim 1, wherein the steroidal lactone is a withanolide isolated from Withania somnifera or a derivative thereof.

3. The method of claim 2, wherein the withanolide is withanolide A.

4. The method of claim 1, wherein the therapeutically effective amount is in the range of 0.2 to 6 mg/kg of body weight.

5. The method of claim 1, wherein the cachexia is induced by a disorder selected from the group consisting of cancer, a neurological disorder, HIV infection, AIDS, sepsis, a chronic pulmonary disease, and a cardiac disorder.

6. The method of claim 1, wherein the therapeutically effective amount of a steroidal lactone is sufficient to activate satellite cells.

7. A method of ameliorating cardiac cachexia in a subject in need thereof comprising administering to the subject a therapeutically effective amount of a steroidal lactone.

8. The method of claim 7, wherein the steroidal lactone is a withanolide isolated from Withania somnifera or a derivative thereof.

9. The method of claim 7, wherein the withanolide is withanolide A.

10. The method of claim 7, wherein the therapeutically effective amount is in the range of 0.2 to 6 mg/kg of body weight.

11. A method of reducing myofibrillar atrophy and/or conversion of type IIA myofibers to type IIB myofibers in a subject in need thereof comprising administering to the subject a therapeutically effective amount of a steroidal lactone.

12. The method of claim 11, wherein the steroidal lactone is a withanolide isolated from Withania somnifera or a derivative thereof.

13. The method of claim 12, wherein the withanolide is withanolide A.

14. The method of claim 11, wherein the therapeutically effective amount is in the range of 0.2 to 6 mg/kg of body weight.

15. The method of claim 11, wherein the subject has cachexia induced by a disorder selected from the group consisting of cancer, a neurological disorder, HIV infection, AIDS, sepsis, a chronic pulmonary disease, and a cardiac disorder.

16. The method of claim 11, wherein the subject has myofibrillar atrophy and/or myofiber-type conversion in skeletal and/or cardiac muscle.

17. The method of claim 11, wherein the therapeutically effective amount of a steroidal lactone is sufficient to activate satellite cells.

18. A method of attenuating arrhythmia, systolic dysfunction, and/or diastolic dysfunction in a subject in need thereof comprising administering to the subject a therapeutically effective amount of a steroidal lactone.

19. The method of claim 18, wherein the subject has cardiac cachexia.

20. The method of claim 18, wherein the steroidal lactone is a withanolide or a derivative thereof.

21. The method of claim 18, wherein the therapeutically effective amount is in the range of 0.2 to 6 mg/kg of body weight.
